(19) United States
(12) Patent Application Publication
(10) Pub. No.: US 2024/0018609 A1
(43) Pub. Date: Jan. 18, 2024

(54) LAMP DETECTION OF SARS-COV-2 IN SALIVA FOR THE RAPID DIAGNOSIS OF COVID-19

(71) Applicant: CITY OF HOPE, Duarte, CA (US)

(72) Inventors: Markus KALKUM, Duarte, CA (US); Sanjeet DADWAL, Duarte, CA (US)

(21) Appl. No.: 18/042,434

(22) PCT Filed: Aug. 18, 2021
(86) PCT No.: PCT/US2021/046569

§ 371 Date: Feb. 21, 2023

Related U.S. Application Data
(60) Provisional application No. 63/067,855, filed on Aug. 19, 2020.

(51) Int. Cl. C12Q 1/70; C12Q 1/6844

(52) U.S. Cl. CPC C12Q 1/701; C12Q 1/6844; C12Q 2600/16

(57) ABSTRACT

Provided herein are, inter alia, kits and methods for the detection SARS-CoV-2 in COVID-19 patients. The kits and methods provided herein are, interalia, useful for the rapid detection of SARS-CoV-2 and related variants in, for example, the saliva of COVID-19 patients.

### CROSS-REFERENCES TO RELATED APPLICATIONS

This application is a national stage filing under U.S.C. 371 of international application PCT/US2021/046569, filed Aug. 18, 2021, which claims priority to U.S. Provisional Application No. 63/067,855, filed Aug. 19, 2020, the disclosures of which are incorporated herein in their entireties and for all purposes.

### REFERENCE TO A “SEQUENCE LISTING,” A TABLE, OR A COMPUTER PROGRAM LISTING APPENDIX SUBMITTED ON A COMPACT DISK

The Sequence Listing written in file 048440-781001WO_SequenceListing_ST25.txt, created on Aug. 18, 2021, 12,288 bytes, machine format IBM-PC, MS-Windows operating system, is hereby incorporated by reference in its entirety for all purposes.

### BACKGROUND

SARS-CoV-2 has caused an unprecedented problem, resulting in unaccountable economic and social loss. Previous epidemics and pandemics included those caused by the Spanish Flu Virus, Avian Flu Virus, SARS (or SAARS-CoV-1), Middle East Respiratory Syndrome (MERS), Zika Virus, and Ebola Virus, etc. a. Provided herein, inter alia, are proposed methods to detect patients infected with SARS-2-CoV, and thus help to prevent or stop the viral spread.

### BRIEF SUMMARY

In an aspect is provided a kit including a set of loop-mediated isothermal amplification (LAMP) primers including: (i) a set of LAMP primers including four primers including nucleic acid sequences having at least 90% sequence identity to the sequences of SEQ ID NO:1, SEQ ID NO:2, SEQ ID NO:3 or SEQ ID NO:4; (ii) a set of LAMP primers including four primers including nucleic acid sequences having at least 90% sequence identity to the sequences of SEQ ID NO:7, SEQ ID NO:8, SEQ ID NO:9 or SEQ ID NO:10; (iii) a set of LAMP primers including four primers including nucleic acid sequences having at least 90% sequence identity to the sequences of SEQ ID NO:13, SEQ ID NO:14, SEQ ID NO:15 or SEQ ID NO:16; (iv) a set of LAMP primers including four primers including nucleic acid sequences having at least 90% sequence identity to the sequences of SEQ ID NO:19, SEQ ID NO:20, SEQ ID NO:21 or SEQ ID NO:22; (v) a set of LAMP primers including four primers including nucleic acid sequences having at least 90% sequence identity to the sequences of SEQ ID NO:25, SEQ ID NO:26, SEQ ID NO:27 or SEQ ID NO:28; or (vi) a set of LAMP primers including four primers including nucleic acid sequences having at least 90% sequence identity to the sequences of SEQ ID NO:31, SEQ ID NO:32, SEQ ID NO:33 or SEQ ID NO:34.

In another aspect is provided a method of detecting a SARS-CoV-2 nucleic acid in a sample, the method including: (a) contacting a sample with a set of loop-mediated isothermal amplification (LAMP) primers under conditions sufficient to amplify a SARS-CoV-2 nucleic acid, wherein the set of LAMP primers includes: (i) a set of LAMP primers including four primers including nucleic acid sequences having at least 90% sequence identity to the sequences of SEQ ID NO:1, SEQ ID NO:2, SEQ ID NO:3 or SEQ ID NO:4; (ii) a set of LAMP primers including four primers including nucleic acid sequences having at least 90% sequence identity to the sequences of SEQ ID NO:7, SEQ ID NO:8, SEQ ID NO:9 or SEQ ID NO:10; (iii) a set of LAMP primers including four primers including nucleic acid sequences having at least 90% sequence identity to the sequences of SEQ ID NO:13, SEQ ID NO:14, SEQ ID NO:15 or SEQ ID NO:16; (iv) a set of LAMP primers including four primers including nucleic acid sequences having at least 90% sequence identity to the sequences of SEQ ID NO:19, SEQ ID NO:20, SEQ ID NO:21 or SEQ ID NO:22; (v) a set of LAMP primers including four primers including nucleic acid sequences having at least 90% sequence identity to the sequences of SEQ ID NO:25, SEQ ID NO:26, SEQ ID NO:27 or SEQ ID NO:28; or (vi) a set of LAMP primers including four primers including nucleic acid sequences having at least 90% sequence identity to the sequences of SEQ ID NO:31, SEQ ID NO:32, SEQ ID NO:33 or SEQ ID NO:34; thereby forming a SARS-CoV-2 amplification product; and (b) detecting the SARS-CoV-2 amplification product, thereby detecting the SARS-CoV-2 nucleic acid in the sample.

### BRIEF DESCRIPTION OF THE DRAWINGS

FIG. 1 presents qRT-PCR amplification curves for saliva samples (technical duplicates). In this figure, NTC: non-template control; POS: positive control; pt01 (patient 01), pt02 (patient 02), pt05 (patient 05) and pt06 (patient 06) are COVID-19 negative patients; pt08 (patient 08), pt14 (patient 14) and pt15 (patient 15) are COVID-19 positive patients.

FIG. 2 presents LAMP amplification curves for positive patient samples with positive and negative controls (technical duplicates) *: primer sequences from Zhang et al. (23).

FIG. 3 presents amplification curves for NTC with different sample matrices, water, human saliva, carrier RNA. * : primer sequences from Zhang et al. (23).

FIG. 4 presents graphs showing the limit of detection of SARS-CoV-2 RNA standard by LAMP with five primer sets. Dashed lines represent mean of Tt values.

FIG. 5 LAMP primer sets for SARS-CoV2. LAMP amplification curves for a COVID-19 positive patient, a COVID-19 negative patient, the EDX respiratory control panel control, and a non-template control (i.e., water).

FIG. 6 LAMP primer sets with SARS-CoV2 variants and controls. LAMP amplification curves for an RNA positive control, EDX Wuhan RNA standard, RNA standards of the SARS-CoV-2 variants alpha (UK), beta (South Africa), gamma (Brazil), and kappa (India) variants, and a non-template control (NTC).

FIG. 7A-7B Relationship between the RT-PCR and LAMP results for saliva samples. FIG. 7 A) Relationship between the mean LAMP time to threshold (Tt) value and the mean RT-PCR cycle threshold (Ct) value. The solid black line represents the fitted regression curve for data points where amplification was observed by both RT-PCR and LAMP, while the dotted black lines represent the 95% confidence interval. The white dots represent saliva samples where the matching NPS sample was negative, while the gray dots represent a positive matching NPS sample. The black dots indicate saliva samples that did not have a matching NPS sample. FIG. 7 B) Ct (RT-PCR) or Tt (RT-LAMP) times for each primer set. Gray bars indicate the mean Ct or Tt value.

### DETAILED DESCRIPTION

### Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

As used herein, the singular forms “a”, “an” and “the” are intended to include the plural forms as well, unless the context clearly indicates otherwise.

In this disclosure, “comprises,” “comprising,” “containing” and “having” and the like can have the meaning ascribed to them in U.S. Patent law and can mean “includes,” “including,” and the like. “Consisting essentially of or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins (1984)).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is amino acids or nucleotides in length.

The terms “corresponding to” and “at a position equivalent to” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue or is “at a position equivalent to” another position when it occupies the same essential structural position within the protein as the given residue. For example, precise amino acid numbering assignments my change between homologous proteins or between versions of the same proteins that differ in length (e.g. due to elimination of a protein domain). Thus, an amino acid residue “at a position equivalent to” another position may be the precise same amino acid position within the context of a given protein domain, but its number assignment may differ due to length between two version of the same protein. An amino acid or nucleotide base “position” is denoted by a number that sequentially identifies each amino acid (or nucleotide base) in the reference sequence based on its position relative to the N-terminus (or 5′-end). As descried above, due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence. By aligning sequences using methods known in the art, a given amino acid position that “corresponds to” or is “equivalent to” a given numbers position is easily identified.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous references, e.g., Current Protocols in Molecular Biology, ed. Ausubel, et al., supra.

The term “probe” or “primer”, as used herein, is defined to be one or more nucleic acid fragments whose specific hybridization to a sample can be detected. A probe or primer can be of any length depending on the particular technique it will be used for. For example, PCR primers are generally between 10 and 40 nucleotides in length, while nucleic acid probes for, e.g., a Southern blot, can be more than a hundred nucleotides in length. The probe may be unlabeled or labeled as described below so that its binding to the target or sample can be detected. The probe can be produced from a source of nucleic acids from one or more particular (preselected) portions of a chromosome, e.g., one or more clones, an isolated whole chromosome or chromosome fragment, or a collection of polymerase chain reaction (PCR) amplification products. The length and complexity of the nucleic acid fixed onto the target element is not critical to the invention. One of skill can adjust these factors to provide optimum hybridization and signal production for a given hybridization procedure, and to provide the required resolution among different genes or genomic locations.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “recombinant” when used with reference, e.g., to a cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “exogenous” refers to a molecule or substance (e.g., a compound, nucleic acid or protein) that originates from outside a given cell or organism. For example, an “exogenous promoter” as referred to herein is a promoter that does not originate from the cell or organism it is expressed by. Conversely, the term “endogenous” or “endogenous promoter” refers to a molecule or substance that is native to, or originates within, a given cell or organism.

“Biological sample” or “sample” refer to materials obtained from or derived from a subject or patient. A biological sample includes sections of tissues such as biopsy and autopsy samples, and frozen sections taken for histological purposes. Such samples include bodily fluids such as saliva, blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

The term “spike protein”, “S protein”, or “SARS-CoV-2 S protein” are used in accordance with their plain meaning as understood in the art and refer to the spike (S) protein of the SARS-CoV-2, or variants or homologs thereof. In embodiments, the S protein is a large (approx. 180 kDa) glycoprotein. In embodiments, the S protein is present on the viral surface as a trimer. The S protein may include two domains, S1 and S2. In embodiments, the S1 domain mediates receptor binding and is divided into two sub-domains, with the N-terminal subdomain (NTD) often binding sialic acid and the C-terminal subdomain (also known as C-domain) binding a specific proteinaceous receptor. In embodiments, the S2 domain mediates viral—membrane fusion through the exposure of a highly conserved fusion peptide. The fusion peptide may be activated through proteolytic cleavage at a site found immediately upstream (S2′), which is common to all coronaviruses. In many (but not all) coronaviruses, additional proteolytic priming may occur at a second site located at the interface of the S1 and S2 domains (S1/S2). In some embodiments, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring the S protein polypeptide (e.g. YP_009724390.1). In embodiments, the S protein is the protein as identified by the NCBI sequence reference YP_009724390.1, homolog or functional fragment thereof.

The terms “virus” or “virus particle” are used according to its plain ordinary meaning within Virology and refers to a virion including the viral genome (e.g. DNA, RNA, single strand, double strand), viral capsid and associated proteins, and in the case of enveloped viruses (e.g. herpesvirus), an envelope including lipids and optionally components of host cell membranes, and/or viral proteins.

The term “replicate” is used in accordance with its plain ordinary meaning and refers to the ability of a cell or virus to produce progeny. A person of ordinary skill in the art will immediately understand that the term replicate when used in connection with DNA, refers to the biological process of producing two identical replicas of DNA from one original DNA molecule. In the context of a virus, the term “replicate” includes the ability of a virus to replicate (duplicate the viral genome and packaging said genome into viral particles) in a host cell and subsequently release progeny viruses from the host cell, which results in the lysis of the host cell.

The terms “SARS-CoV-2” or “SARS-CoV 2” refer to the Severe Acute Respiratory Syndrome Coronavirus 2. In embodiments, the SARS-CoV-2 is the strain of coronavirus that causes coronavirus disease 2019 (COVID-19), the respiratory illness responsible for the COVID-19 pandemic. In embodiments, the SARS-CoV-2 is colloquially known as simply the coronavirus, it was previously referred to by its provisional name, 2019 novel coronavirus (2019-nCoV), and has also been called human coronavirus 2019 (HCoV-19 or hCoV-19). In embodiments, the SARS-CoV-2 is a Baltimore class IV positive-sense single-stranded RNA virus that is contagious in humans.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “effective dose” or “effective dosage” is defined as an amount sufficient to achieve or at least partially achieve the desired effect.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

An “inhibitor” refers to a compound (e.g. compounds described herein) that reduces activity when compared to a control, such as absence of the compound or a compound with known inactivity.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

The term “contacting” may include allowing two species to react, interact, or

physically touch, wherein the two species may be a compound as described herein and a protein or enzyme. In some embodiments contacting includes allowing a compound described herein to interact with a protein or enzyme that is involved in a signaling pathway.

As defined herein, the term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “associated” or “associated with” in the context of a substance or substance activity or function associated with a disease (e.g. a protein associated disease, a cancer (e.g., cancer, inflammatory disease, autoimmune disease, or infectious disease)) means that the disease (e.g. cancer, inflammatory disease, autoimmune disease, or infectious disease) is caused by (in whole or in part), or a symptom of the disease is caused by (in whole or in part) the substance or substance activity or function. As used herein, what is described as being associated with a disease, if a causative agent, could be a target for treatment of the disease.

A “control” or “standard control” refers to a sample, measurement, or value that serves as a reference, usually a known reference, for comparison to a test sample, measurement, or value. For example, a test sample can be taken from a patient suspected of having a given disease (e.g. cancer) and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. RNA levels, protein levels, specific cell types, specific bodily fluids, specific tissues, synoviocytes, synovial fluid, synovial tissue, fibroblast-like synoviocytes, macrophagelike synoviocytes, etc).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

“Patient” or “subject in need thereof” refers to a living organism suffering from or

prone to a disease or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “COVID19”, “COVID-19” refer to the coronavirus disease 2019, caused by SARS-CoV-2. In embodiments, the COVID-19 is a respiratory illness characterized by symptoms such as fever, cough, loss of appetite, fatigue, shortness of breath, coughing up sputum, muscle aches and pains, nausea, vomiting, diarrhea, sneezing, runny nose, sore throat, skin lesions, chest tightness, palpitations, decrease sense or loss of sense of smell, and/or disturbances in sense of taste. Comorbidities of COVID-19 include moderate or severe asthma, pre-existing chronic obstructive pulmonary disease, pulmonary fibrosis, cystic fibrosis, hypertension, diabetes mellitus, and cardiovascular diseases such as, but limited to, coronary artery diseases, stroke, heart failure, hypertensive heart disease, rheumatic heart disease, or cardiomyopathy.

The terms “disease” or “condition” refer to a state of being or health status of a patient or subject capable of being treated with the compounds or methods provided herein.

The term “prophylactic treatment” as used herein, refers to any intervention using the compositions embodied herein, that is administered to an individual in need thereof or having an increased risk of acquiring a respiratory tract infection, wherein the intervention is carried out prior to the onset of a viral infection, e.g. SARS-CoV-2, and typically has in effect that either no viral infection occurs or no clinically relevant symptoms of a viral infection occur in a healthy individual upon subsequent exposure to an amount of infectious viral agent that would otherwise, i.e. in the absence of such a prophylactic treatment, be sufficient to cause a viral infection.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

By “therapeutically effective dose or amount” as used herein is meant a dose that produces effects for which it is administered (e.g. treating or preventing a disease such as COVID-19 and its implications). The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a standard control. A therapeutically effective dose or amount may ameliorate one or more symptoms of a disease. A therapeutically effective dose or amount may prevent or delay the onset of a disease or one or more symptoms of a disease when the effect for which it is being administered is to treat a person who is at risk of developing the disease.

The term “therapy” or “therapeutic treatment” as used herein relates to the administration of the compositions embodied herein, in order to achieve a reduction in severity and/or frequency of symptoms, elimination of symptoms and/or underlying cause, prevention of the occurrence of symptoms and/or their underlying cause, and/or improvement or remediation of damage directly caused by or indirectly associated, e.g. through secondary infection, with the viral infection.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

“Treatment”, or “treating” as used herein, is defined as the application or administration of a therapeutic agent or combination of therapeutic agents to a patient, or application or administration of the active agent to a patient, who has a virus infection, e.g. SARS-CoV-2, with the purpose to cure, heal, alleviate, relieve, alter, remedy, ameliorate, improve or affect the infection, or symptoms thereof. The term “treatment” or “treating” is also used herein in the context of administering agents prophylactically. Accordingly, “treating” or “treatment” of a state, disorder or condition includes: (1) eradicating the virus; (2) preventing or delaying the appearance of clinical symptoms of the state, disorder or condition developing in a human or other mammal that may be afflicted with or predisposed to the state, disorder or condition but does not yet experience or display clinical or subclinical symptoms of the state, disorder or condition; (3) inhibiting the state, disorder or condition, i.e., arresting, reducing or delaying the development of the disease or a relapse thereof (in case of maintenance treatment) or at least one clinical or subclinical symptom thereof; or (4) relieving the disease, i.e., causing regression of the state, disorder or condition or at least one of its clinical or subclinical symptoms. The benefit to an individual to be treated is either statistically significant or at least perceptible to the patient or to the physician.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and. This applies regardless of the breadth of the range.

Any compositions or methods provided herein can be combined with one or more of any of the other compositions and methods provided herein.

A number of embodiments have been described. Nevertheless, it will be understood that various modifications may be made. Accordingly, other embodiments are within the scope of the claims.

The term “amplification” as provided herein refers to increasing the number of copies of a nucleic acid molecule, such as a gene or fragment of a gene, for example at least a portion of a SARS-CoV-2 nucleic acid molecule. The products of an amplification reaction are referred to herein as “amplification products.” A non-limiting example of in vitro amplification is the polymerase chain reaction (PCR), in which a sample (such as a biological sample from a subject) is contacted with a pair of oligonucleotide primers, under conditions that allow for hybridization of the primers to a nucleic acid molecule in the sample. The primers are extended under suitable conditions, dissociated from the template, and then re-annealed, extended, and dissociated to amplify the number of copies of the nucleic acid molecule. Other examples of in vitro amplification techniques include real-time PCR, quantitative real-time PCR (qPCR), reverse transcription PCR (RT-PCR), quantitative RT-PCR (qRT-PCR), loop-mediated isothermal amplification (LAMP; see Notomi et al., Nucl. Acids Res. 28:e63, 2000); reverse-transcriptase LAMP (RT-LAMP); strand displacement amplification (see, for example, U.S. Pat. No. 5,744,311); transcription-free isothermal amplification (see, for example, U.S. Pat. No. 6,033,881); repair chain reaction amplification (see, for example, WO 90/01069); ligase chain reaction amplification (see, for example, EP-A-320 308); gap filling ligase chain reaction amplification (see, for example, U.S. Pat. No. 5,427,930); coupled ligase detection and PCR (see, for example, U.S. Pat. No. 6,027,889); and NASBA™ RNA transcription-free amplification (see, for example, U.S. Pat. No. 6,025,134).

The phrase “conditions sufficient for” as provided herein refer to any environment that allows the desired activity, for example, that allows specific binding or hybridization between two nucleic acid molecules or that allows reverse transcription and/or amplification of a nucleic acid. Such an environment may include, without limitation, particular incubation conditions (such as time and/or temperature) or presence and/or concentration of particular factors, for example in a solution (such as, e.g., buffer(s), salt(s), metal ion(s), detergent(s), nucleotide(s), enzyme(s)).

The term “loop-mediated isothermal amplification” or “LAMP” as provided herein refers to a method of amplifying DNA including a single-step amplification reaction utilizing a DNA polymerase with strand displacement capabilities (e.g., a polymerase as described in Notomi et al., Nucl. Acids. Res. 28:E63, 2000; Nagamine et al., Mol. Cell. Probes 16:223-229, 2002; or Mori et al., J. Biochem. Biophys. Methods 59:145-157, 2004). At least four primers, which are specific for eight regions within a target nucleic acid sequence, are typically used for LAMP. The primers include a forward outer primer (F3), a reverse outer primer (R3), a forward inner primer (FTP), and a reverse inner primer (RIP). A forward loop primer (LF), and a reverse loop primer (LR) may also be included in embodiments. The amplification reaction forms a stem-loop DNA with inverted repeats of the target nucleic acid sequence. In embodiments, reverse transcriptase is added to the reaction for amplification of RNA target sequences. Where reverse transcriptase is added to the reaction the amplification is referred to as RT-LAMP.

The term “primer” or “primers” as referred to herein are short nucleic acid molecules, generally DNA oligonucleotides with 10 nucleotides or more in length (e.g., 12, 18, 20, 25, 30, 35, 40, or more nucleotides in length). Primers may be annealed to a complementary target DNA strand by nucleic acid hybridization to form a hybrid molecule between the primer and the target DNA strand. Subsequently the primer may be extended by a DNA polymerase enzyme and using the target DNA strand as template. In embodiments, primer pairs are used for amplification of a nucleic acid sequence, e.g., by the polymerase chain reaction (PCR) or other nucleic acid amplification methods known in the art and described herein.

A “sample” or “biological sample” as provided herein refers to a biological specimen including DNA (e.g., genomic DNA or cDNA), RNA (e.g., mRNA), protein, or combinations thereof. Non-limiting examples of samples include, isolated nucleic acids, cells, cell lysates, chromosomal preparations, peripheral blood, serum, plasma, urine, saliva, tissue biopsy, surgical specimen, bone marrow, amniocentesis samples, and autopsy material. In embodiments, a sample includes viral nucleic acids, for example, viral DNA, viral RNA, or cDNA reverse transcribed from viral RNA. In embodiments, samples are used directly (e.g., fresh or frozen), or can be manipulated prior to use, for example, by heat-treatment, purification of nucleic acids, fixation (e.g., using formalin) and/or embedding in wax (such as FFPE tissue samples). In embodiments, a sample is a saliva sample.

### Compositions

The methods and compositions provided herein may, inter alia, be used for detection of viral nucleic acids (e.g., SARS-CoV-2 nucleic acids), including diagnostic and prognostic applications, such as in laboratory and clinical settings. Appropriate samples include any conventional biological samples, including clinical samples obtained from a human or veterinary subject. Suitable samples include all biological samples useful for detection of infection in subjects, including, but not limited to, cells, tissues, autopsy samples, bone marrow aspirates, bodily fluids (for example, blood, serum, plasma, urine, cerebrospinal fluid, middle ear fluids, bronchoalveolar lavage, tracheal aspirates, sputum, nasopharyngeal aspirates, oropharyngeal aspirates, or saliva), oral swabs, nasal swaps, eye swabs, cervical swabs, vaginal swabs, rectal swabs, stool, and stool suspensions. The sample can be used directly or can be processed, such as by adding solvents, preservatives, buffers, or other compounds or substances. In embodiments, the nucleic acids are isolated from the sample. In embodiments, isolation of nucleic acids from the sample is not necessary prior to use in the methods disclosed herein and the sample (e.g., saliva) is used directly (without nucleic acid extraction). In embodiments, the sample is pre-treated with a lysis buffer, without isolating the nucleic acids for use in the disclosed methods.

Samples may also include isolated nucleic acids, such as DNA or RNA isolated from a biological specimen from a subject, a viral isolate, or other source of nucleic acids. Methods for extracting nucleic acids such as RNA or DNA from a sample are known to one of skill in the art; such methods will depend upon, for example, the type of sample in which the nucleic acid is found. Nucleic acids can be extracted using standard methods. For example, rapid nucleic acid preparation can be performed using a commercially available kit (such as kits and/or instruments from Qiagen (such as QiaAmpO, DNEasy® or RNEasy® kits), Roche Applied Science (such as MagNA Pure kits and instruments), Thermo Scientific (KingFisher mL), bioMerieux (Nuclisens® NASBA Diagnostics), or Epicentre (Masterpure™ kits)). In embodiments, the nucleic acids may be extracted using guanidinium isothiocyanate, such as single-step isolation by acid guanidinium isothiocyanate-phenol-chloroform extraction (Chomczynski et al. Anal. Biochem. 162:156-159, 1987).

The methods and compositions provided herein including embodiments thereof are, inter alia, highly sensitive and/or specific for detection of SARS-CoV-2 nucleic acids. In embodiments, the methods detect the presence of at least 1 International Unit (IU; about 5 copies) of SARS-CoV-2 nucleic acids (for example at least 10, 25, 50, 10², 10⁴ 10⁵, 10⁶, or more IU of SARS-CoV-2 nucleic acids) in a sample or reaction volume. In embodiments, the methods and compositions provided herein including embodiments thereof, are used for the detection of the presence of at least 1 copy of SARS-CoV-2 nucleic acids (for example at least 10, 25, 50, 10², 10⁴ 10⁵, 10⁶, or more copies) in a sample or reaction volume. In embodiments, the methods and compositions provided herein including embodiments thereof, predict with a sensitivity of at least 75% and a specificity of at least 75% for presence of SARS-CoV-2 nucleic acids in a sample, such as a sensitivity of at least 80%, 85%, 90%, 95%, or even 100% and a specificity of at least of at least 80%, 85%, 90%, 95%, or even 100%.

In embodiments, the methods for detecting viral nucleic acids in a sample utilize LAMP or RT-LAMP methods of amplification and detection. As described above, LAMP is a one-step isothermal amplification method that may produce amplified nucleic acids in a short period of time using a DNA polymerase with strand displacement activity (see, e.g., Notomi et al., Nucl. Acids Res. 28:e63, 2000). LAMP may be used for amplification of RNA targets with the addition of reverse transcriptase (RT) to the reaction without an additional heat step (referred to as RT-LAMP). The isothermal nature of LAMP and RT-LAMP allows for assay flexibility because it may be used with simple and inexpensive heating devices, which can facilitate viral detection in settings other than centralized clinical laboratories. In addition, LAMP and RT-LAMP assays are rapid, specific, and sensitive.

In embodiments, the sample and LAMP primer set(s) are contacted under conditions sufficient for amplification of a viral nucleic acid(s), producing an amplification product. The sample is contacted with the set(s) of LAMP primers at a concentration sufficient to support amplification of the SARS-CoV-2 nucleic acid(s) for the LAMP primer set(s). In embodiments, the amount of each primer is about 0.1 μM to about 5 μM (such as about 0.2 μM to about 2 μM, or about 0.5 μM to about 2 μM). Each primer may be included at a different concentration, and appropriate concentrations for each primer may be selected by one of skill in the art using routine methods. Exemplary primer concentrations are provided herein (e.g., Examples section).

In embodiments, the LAMP or RT-LAMP reaction is carried out in a mixture including a suitable buffer (such as a phosphate buffer or Tris buffer). The buffer may also include additional components, such as salts (such as KCl or NaCl, magnesium salts (e.g., MgCl₂ or MgSO₄), ammonium (e.g., (NH₄)₂SO₄)), detergents (e.g., TRITON-X100), or other additives (such as betaine or dimethylsulfoxide). The buffer or reaction mixture also includes nucleotides or nucleotide analogs. In embodiments, an equimolar mixture of dATP, dCTP, dGTP, and dTTP (referred to as dNTPs) is included, for example about 0.5-5 mM dNTPs (such as about 1-3 mM dNTPs). Any appropriate buffer and any additives using routine methods is contemplated for the methods provided herein.

The polymerase provided herein may be a DNA polymerase with strand displacement activity. Non-limiting examples of DNA polymerases include Bst DNA polymerase, Phi29 DNA polymerase, Bsu DNA polymerase, Taq DNA polymerase, Klenow fragment of DNA polymerase I, PhiPRD1 DNA polymerase, phage M2 DNA polymerase, T4 DNA polymerase, and T5 DNA polymerase. In embodiments, about 1 to 20 Units (U) (such as about 1 to 15 U, about 2 to 12 U, about 10 to 20 U, about 2 to 10 U, about 5 to 10 U, or 8 U) of DNA polymerase is included in the reaction. In SARS-CoV-2, the polymerase has strand displacement activity and lacks 5′-3′ exonuclease activity. In embodiments, the DNA polymerase is Bst DNA polymerase.

The reaction mixture, including sample, LAMP primers, buffers, nucleotides, DNA polymerase, and any other components, may be incubated for a period of time and at a temperature sufficient for production of an amplification product. In embodiments, the reaction conditions include incubating the reaction mixture at about 37° C. to about 80° C. (such as about 40° C. to about 70° C., about 50° C. to about 65° C., or about 60° C. to about 65° C.), for example about 40° C., about 45° C., about 50° C., about 55° C., about 60° C., about 65° C., about about 75° C., or about 80° C. In embodiments, the reaction mixture is incubated at about 60°, 63°, or 65°. The reaction mixture may be incubated for at least about 5 minutes (such as about 10, about 15, about 20, about 30, about 40, about 50, about 60, about 70, about 80 about 90, about 100, about 110, about 120 minutes or more), for example about 10-120 minutes, about 15-90 minutes, about 20-70 minutes, or about 30-60 minutes.

Following incubation of the reaction mixture, the amplification product is detected by any suitable method. The detection methods may be quantitative, semi-quantitative, or qualitative. In embodiments, accumulation of an amplification product is detected by measuring the turbidity of the reaction mixture (for example, visually or with a turbidometer). In embodiments, amplification product is detected using gel electrophoresis, for example by detecting presence or amount of amplification product with agarose gel electrophoresis. In embodiments, amplification product is detected using a colorimetric assay, such as with an intercalating dye (for example, propidium iodide, SYBRO green, GelRed™, or GelGreen™ dyes). In further embodiments, amplification product is detected with a metal ion sensitive fluorescent molecule (for example, calcein, which is a fluorescence dye that is quenched by manganese ions and has increased fluorescence when bound to magnesium ions). In embodiments, amplification products are detected using a detectable label incorporated in one or more of the LAMP primers (described herein). The detectable label may be optically detectable, for example, by eye or using a spectrophotometer or fluorimeter. In embodiments, the detectable label is a fluorophore. In embodiments, the label is detected using a fluorescence scanner (such as ESEQuant Tube Scanner, Qiagen; NanoDrop™ 3300 Fluorospectrometer, Thermo Scientific). One of skill in the art will be able to select one or more detectable labels for use in the methods and compositions disclosed herein.

In embodiments, the disclosed methods include detecting fluorescence from a detectable label incorporated in one or more LAMP primers. In embodiments, the sample is identified as containing a viral nucleic acid (for example is “positive” for the virus) if an increase in fluorescence is detected relative to a control (such as a no template control sample or a known negative sample). In embodiments, the amount of viral nucleic acid in a sample is determined semi-quantitatively or quantitatively. For example, the amount of viral nucleic acid in a test sample may be determined by comparing the amount of fluorescence obtained in a LAMP assay with fluorescence obtained in a LAMP assay with samples containing known amounts of the viral nucleic acid of interest or a standard curve prepared from such samples.

In embodiments, one of the LAMP primers in a set includes a detectable label, such as a fluorophore. In embodiments, a LAMP primer including a detectable label may be referred to herein as a “probe.” In a specific example, an LR primer (e.g., a primer having the sequence of SEQ ID NOs: 1-37) includes a fluorophore, for example attached to the 5′ end or the 3′ end of the primer. Any fluorophore may be used; in some non-limiting examples, the fluorophore is TET, FAM, Cy3, or TexasRed. In embodiments, the labeled LAMP primer includes an acceptor fluorophore (a quencher).

### Kit Compositions

In an aspect is provided a kit including a set of loop-mediated isothermal amplification (LAMP) primers including: (i) a set of LAMP primers including four primers including nucleic acid sequences having at least 90% sequence identity to the sequences of SEQ ID NO:1, SEQ ID NO:2, SEQ ID NO:3 or SEQ ID NO:4; (ii) a set of LAMP primers including four primers including nucleic acid sequences having at least 90% sequence identity to the sequences of SEQ ID NO:7, SEQ ID NO:8, SEQ ID NO:9 or SEQ ID NO:10; (iii) a set of LAMP primers including four primers including nucleic acid sequences having at least 90% sequence identity to the sequences of SEQ ID NO:13, SEQ ID NO:14, SEQ ID NO:15 or SEQ ID NO:16; (iv) a set of LAMP primers including four primers including nucleic acid sequences having at least 90% sequence identity to the sequences of SEQ ID NO:19, SEQ ID NO:20, SEQ ID NO:21 or SEQ ID NO:22; (v) a set of LAMP primers including four primers including nucleic acid sequences having at least 90% sequence identity to the sequences of SEQ ID NO:25, SEQ ID NO:26, SEQ ID NO:27 or SEQ ID NO:28; or (vi) a set of LAMP primers including four primers including nucleic acid sequences having at least 90% sequence identity to the sequences of SEQ ID NO:31, SEQ ID NO:32, SEQ ID NO:33 or SEQ ID NO:34.

In an aspect is provided a kit including a set of loop-mediated isothermal amplification (LAMP) primers including: (i) a set of LAMP primers including four primers including nucleic acid sequences having at least 90% sequence identity to the sequences of SEQ ID NO:1, SEQ ID NO:2, SEQ ID NO:3 or SEQ ID NO:4; (ii) a set of LAMP primers including four primers including nucleic acid sequences having at least 90% sequence identity to the sequences of SEQ ID NO:7, SEQ ID NO:8, SEQ ID NO:9 or SEQ ID NO:10; (iii) a set of LAMP primers including four primers including nucleic acid sequences having at least 90% sequence identity to the sequences of SEQ ID NO:13, SEQ ID NO:14, SEQ ID NO:15 or SEQ ID NO:16; (iv) a set of LAMP primers including four primers including nucleic acid sequences having at least 90% sequence identity to the sequences of SEQ ID NO:19, SEQ ID NO:20, SEQ ID NO:21 or SEQ ID NO:22; (v) a set of LAMP primers including four primers including nucleic acid sequences having at least 90% sequence identity to the sequences of SEQ ID NO:25, SEQ ID NO:26, SEQ ID NO:27 or SEQ ID NO:28; or (vi) a set of LAMP primers including four primers including nucleic acid sequences having at least 90% sequence identity to the sequences of SEQ ID NO:31, SEQ ID NO:32, SEQ ID NO:37 or SEQ ID NO:34.

In an aspect is provided a kit including a set of loop-mediated isothermal amplification (LAMP) primers including: (i) a set of LAMP primers including four primers including nucleic acid sequences having 90% sequence identity to the sequences of SEQ ID NO:1, SEQ ID NO:2, SEQ ID NO:3 or SEQ ID NO:4; (ii) a set of LAMP primers including four primers including nucleic acid sequences having 90% sequence identity to the sequences of SEQ ID NO:7, SEQ ID NO:8, SEQ ID NO:9 or SEQ ID NO:10; (iii) a set of LAMP primers including four primers including nucleic acid sequences having 90% sequence identity to the sequences of SEQ ID NO:13, SEQ ID NO:14, SEQ ID NO:15 or SEQ ID NO:16; (iv) a set of LAMP primers including four primers including nucleic acid sequences having 90% sequence identity to the sequences of SEQ ID NO:19, SEQ ID NO:20, SEQ ID NO:21 or SEQ ID NO:22; (v) a set of LAMP primers including four primers including nucleic acid sequences having 90% sequence identity to the sequences of SEQ ID NO:25, SEQ ID NO:26, SEQ ID NO:27 or SEQ ID NO:28; or (vi) a set of LAMP primers including four primers including nucleic acid sequences having 90% sequence identity to the sequences of SEQ ID NO:31, SEQ ID NO:32, SEQ ID NO:33 or SEQ ID NO:34.

In an aspect is provided a kit including a set of loop-mediated isothermal amplification (LAMP) primers including: (i) a set of LAMP primers including four primers including nucleic acid sequences having 90% sequence identity to the sequences of SEQ ID NO:1, SEQ ID NO:2, SEQ ID NO:3 or SEQ ID NO:4; (ii) a set of LAMP primers including four primers including nucleic acid sequences having 90% sequence identity to the sequences of SEQ ID NO:7, SEQ ID NO:8, SEQ ID NO:9 or SEQ ID NO:10; (iii) a set of LAMP primers including four primers including nucleic acid sequences having 90% sequence identity to the sequences of SEQ ID NO:13, SEQ ID NO:14, SEQ ID NO:15 or SEQ ID NO:16; (iv) a set of LAMP primers including four primers including nucleic acid sequences having 90% sequence identity to the sequences of SEQ ID NO:19, SEQ ID NO:20, SEQ ID NO:21 or SEQ ID NO:22; (v) a set of LAMP primers including four primers including nucleic acid sequences having 90% sequence identity to the sequences of SEQ ID NO:25, SEQ ID NO:26, SEQ ID NO:27 or SEQ ID NO:28; or (vi) a set of LAMP primers including four primers including nucleic acid sequences having 90% sequence identity to the sequences of SEQ ID NO:31, SEQ ID NO:32, SEQ ID NO:37 or SEQ ID NO:34.

In embodiments, the set of loop-mediated isothermal amplification (LAMP) primers includes: (i) a set of LAMP primers including four primers including nucleic acid sequences having at least 95% sequence identity to the sequences of SEQ ID NO:1, SEQ ID NO:2, SEQ ID NO:3 or SEQ ID NO:4; (ii) a set of LAMP primers including four primers including nucleic acid sequences having at least 95% sequence identity to the sequences of SEQ ID NO:7, SEQ ID NO:8, SEQ ID NO:9 or SEQ ID NO:10; (iii) a set of LAMP primers including four primers including nucleic acid sequences having at least 95% sequence identity to the sequences of SEQ ID NO:13, SEQ ID NO:14, SEQ ID NO:15 or SEQ ID NO:16; (iv) a set of LAMP primers including four primers including nucleic acid sequences having at least 95% sequence identity to the sequences of SEQ ID NO:19, SEQ ID NO:20, SEQ ID NO:21 or SEQ ID NO:22; (v) a set of LAMP primers including four primers including nucleic acid sequences having at least 95% sequence identity to the sequences of SEQ ID NO:25, SEQ ID NO:26, SEQ ID NO:27 or SEQ ID NO:28; or (vi) a set of LAMP primers including four primers including nucleic acid sequences having at least 95% sequence identity to the sequences of SEQ ID NO:31, SEQ ID NO:32, SEQ ID NO:33 or SEQ ID NO:34.

In embodiments, the set of loop-mediated isothermal amplification (LAMP) primers includes: (i) a set of LAMP primers including four primers including nucleic acid sequences having at least 95% sequence identity to the sequences of SEQ ID NO:1, SEQ ID NO:2, SEQ ID NO:3 or SEQ ID NO:4; (ii) a set of LAMP primers including four primers including nucleic acid sequences having at least 95% sequence identity to the sequences of SEQ ID NO:7, SEQ ID NO:8, SEQ ID NO:9 or SEQ ID NO:10; (iii) a set of LAMP primers including four primers including nucleic acid sequences having at least 95% sequence identity to the sequences of SEQ ID NO:13, SEQ ID NO:14, SEQ ID NO:15 or SEQ ID NO:16; (iv) a set of LAMP primers including four primers including nucleic acid sequences having at least 95% sequence identity to the sequences of SEQ ID NO:19, SEQ ID NO:20, SEQ ID NO:21 or SEQ ID NO:22; (v) a set of LAMP primers including four primers including nucleic acid sequences having at least 95% sequence identity to the sequences of SEQ ID NO:25, SEQ ID NO:26, SEQ ID NO:27 or SEQ ID NO:28; or (vi) a set of LAMP primers including four primers including nucleic acid sequences having at least 95% sequence identity to the sequences of SEQ ID NO:31, SEQ ID NO:32, SEQ ID NO:37 or SEQ ID NO:34.

In embodiments, the set of loop-mediated isothermal amplification (LAMP) primers includes: (i) a set of LAMP primers including four primers including nucleic acid sequences having 95% sequence identity to the sequences of SEQ ID NO:1, SEQ ID NO:2, SEQ ID NO:3 or SEQ ID NO:4; (ii) a set of LAMP primers including four primers including nucleic acid sequences having 95% sequence identity to the sequences of SEQ ID NO:7, SEQ ID NO:8, SEQ ID NO:9 or SEQ ID NO:10; (iii) a set of LAMP primers including four primers including nucleic acid sequences having 95% sequence identity to the sequences of SEQ ID NO:13, SEQ ID NO:14, SEQ ID NO:15 or SEQ ID NO:16; (iv) a set of LAMP primers including four primers including nucleic acid sequences having 95% sequence identity to the sequences of SEQ ID NO:19, SEQ ID NO:20, SEQ ID NO:21 or SEQ ID NO:22; (v) a set of LAMP primers including four primers including nucleic acid sequences having 95% sequence identity to the sequences of SEQ ID NO:25, SEQ ID NO:26, SEQ ID NO:27 or SEQ ID NO:28; or (vi) a set of LAMP primers including four primers including nucleic acid sequences having 95% sequence identity to the sequences of SEQ ID NO:31, SEQ ID NO:32, SEQ ID NO:33 or SEQ ID NO:34.

In embodiments, the set of loop-mediated isothermal amplification (LAMP) primers includes: (i) a set of LAMP primers including four primers including nucleic acid sequences having 95% sequence identity to the sequences of SEQ ID NO:1, SEQ ID NO:2, SEQ ID NO:3 or SEQ ID NO:4; (ii) a set of LAMP primers including four primers including nucleic acid sequences having 95% sequence identity to the sequences of SEQ ID NO:7, SEQ ID NO:8, SEQ ID NO:9 or SEQ ID NO:10; (iii) a set of LAMP primers including four primers including nucleic acid sequences having 95% sequence identity to the sequences of SEQ ID NO:13, SEQ ID NO:14, SEQ ID NO:15 or SEQ ID NO:16; (iv) a set of LAMP primers including four primers including nucleic acid sequences having 95% sequence identity to the sequences of SEQ ID NO:19, SEQ ID NO:20, SEQ ID NO:21 or SEQ ID NO:22; (v) a set of LAMP primers including four primers including nucleic acid sequences having 95% sequence identity to the sequences of SEQ ID NO:25, SEQ ID NO:26, SEQ ID NO:27 or SEQ ID NO:28; or (vi) a set of LAMP primers including four primers including nucleic acid sequences having 95% sequence identity to the sequences of SEQ ID NO:31, SEQ ID NO:32, SEQ ID NO:37 or SEQ ID NO:34.

In embodiments, the set of loop-mediated isothermal amplification (LAMP) primers includes: (i) a set of LAMP primers including four primers including nucleic acid sequences having at least 98% sequence identity to the sequences of SEQ ID NO:1, SEQ ID NO:2, SEQ ID NO:3 or SEQ ID NO:4; (ii) a set of LAMP primers including four primers including nucleic acid sequences having at least 98% sequence identity to the sequences of SEQ ID NO:7, SEQ ID NO:8, SEQ ID NO:9 or SEQ ID NO:10; (iii) a set of LAMP primers including four primers including nucleic acid sequences having at least 98% sequence identity to the sequences of SEQ ID NO:13, SEQ ID NO:14, SEQ ID NO:15 or SEQ ID NO:16; (iv) a set of LAMP primers including four primers including nucleic acid sequences having at least 98% sequence identity to the sequences of SEQ ID NO:19, SEQ ID NO:20, SEQ ID NO:21 or SEQ ID NO:22; (v) a set of LAMP primers including four primers including nucleic acid sequences having at least 98% sequence identity to the sequences of SEQ ID NO:25, SEQ ID NO:26, SEQ ID NO:27 or SEQ ID NO:28; or (vi) a set of LAMP primers including four primers including nucleic acid sequences having at least 98% sequence identity to the sequences of SEQ ID NO:31, SEQ ID NO:32, SEQ ID NO:33 or SEQ ID NO:34.

In embodiments, the set of loop-mediated isothermal amplification (LAMP) primers includes: (i) a set of LAMP primers including four primers including nucleic acid sequences having at least 98% sequence identity to the sequences of SEQ ID NO:1, SEQ ID NO:2, SEQ ID NO:3 or SEQ ID NO:4; (ii) a set of LAMP primers including four primers including nucleic acid sequences having at least 98% sequence identity to the sequences of SEQ ID NO:7, SEQ ID NO:8, SEQ ID NO:9 or SEQ ID NO:10; (iii) a set of LAMP primers including four primers including nucleic acid sequences having at least 98% sequence identity to the sequences of SEQ ID NO:13, SEQ ID NO:14, SEQ ID NO:15 or SEQ ID NO:16; (iv) a set of LAMP primers including four primers including nucleic acid sequences having at least 98% sequence identity to the sequences of SEQ ID NO:19, SEQ ID NO:20, SEQ ID NO:21 or SEQ ID NO:22; (v) a set of LAMP primers including four primers including nucleic acid sequences having at least 98% sequence identity to the sequences of SEQ ID NO:25, SEQ ID NO:26, SEQ ID NO:27 or SEQ ID NO:28; or (vi) a set of LAMP primers including four primers including nucleic acid sequences having at least 98% sequence identity to the sequences of SEQ ID NO:31, SEQ ID NO:32, SEQ ID NO:37 or SEQ ID NO:34.

In embodiments, the set of loop-mediated isothermal amplification (LAMP) primers includes: (i) a set of LAMP primers including four primers including nucleic acid sequences having 98% sequence identity to the sequences of SEQ ID NO:1, SEQ ID NO:2, SEQ ID NO:3 or SEQ ID NO:4; (ii) a set of LAMP primers including four primers including nucleic acid sequences having 98% sequence identity to the sequences of SEQ ID NO:7, SEQ ID NO:8, SEQ ID NO:9 or SEQ ID NO:10; (iii) a set of LAMP primers including four primers including nucleic acid sequences having 98% sequence identity to the sequences of SEQ ID NO:13, SEQ ID NO:14, SEQ ID NO:15 or SEQ ID NO:16; (iv) a set of LAMP primers including four primers including nucleic acid sequences having 98% sequence identity to the sequences of SEQ ID NO:19, SEQ ID NO:20, SEQ ID NO:21 or SEQ ID NO:22; (v) a set of LAMP primers including four primers including nucleic acid sequences having 98% sequence identity to the sequences of SEQ ID NO:25, SEQ ID NO:26, SEQ ID NO:27 or SEQ ID NO:28; or (vi) a set of LAMP primers including four primers including nucleic acid sequences having 98% sequence identity to the sequences of SEQ ID NO:31, SEQ ID NO:32, SEQ ID NO:33 or SEQ ID NO:34.

In embodiments, the set of loop-mediated isothermal amplification (LAMP) primers includes: (i) a set of LAMP primers including four primers including nucleic acid sequences having 98% sequence identity to the sequences of SEQ ID NO:1, SEQ ID NO:2, SEQ ID NO:3 or SEQ ID NO:4; (ii) a set of LAMP primers including four primers including nucleic acid sequences having 98% sequence identity to the sequences of SEQ ID NO:7, SEQ ID NO:8, SEQ ID NO:9 or SEQ ID NO:10; (iii) a set of LAMP primers including four primers including nucleic acid sequences having 98% sequence identity to the sequences of SEQ ID NO:13, SEQ ID NO:14, SEQ ID NO:15 or SEQ ID NO:16; (iv) a set of LAMP primers including four primers including nucleic acid sequences having 98% sequence identity to the sequences of SEQ ID NO:19, SEQ ID NO:20, SEQ ID NO:21 or SEQ ID NO:22; (v) a set of LAMP primers including four primers including nucleic acid sequences having 98% sequence identity to the sequences of SEQ ID NO:25, SEQ ID NO:26, SEQ ID NO:27 or SEQ ID NO:28; or (vi) a set of LAMP primers including four primers including nucleic acid sequences having 98% sequence identity to the sequences of SEQ ID NO:31, SEQ ID NO:32, SEQ ID NO:37 or SEQ ID NO:34.

In embodiments, the set of loop-mediated isothermal amplification (LAMP) primers includes: (i) a set of LAMP primers including four primers including nucleic acid sequences having at least 99% sequence identity to the sequences of SEQ ID NO:1, SEQ ID NO:2, SEQ ID NO:3 or SEQ ID NO:4; (ii) a set of LAMP primers including four primers including nucleic acid sequences having at least 99% sequence identity to the sequences of SEQ ID NO:7, SEQ ID NO:8, SEQ ID NO:9 or SEQ ID NO:10; (iii) a set of LAMP primers including four primers including nucleic acid sequences having at least 99% sequence identity to the sequences of SEQ ID NO:13, SEQ ID NO:14, SEQ ID NO:15 or SEQ ID NO:16; (iv) a set of LAMP primers including four primers including nucleic acid sequences having at least 99% sequence identity to the sequences of SEQ ID NO:19, SEQ ID NO:20, SEQ ID NO:21 or SEQ ID NO:22; (v) a set of LAMP primers including four primers including nucleic acid sequences having at least 99% sequence identity to the sequences of SEQ ID NO:25, SEQ ID NO:26, SEQ ID NO:27 or SEQ ID NO:28; or (vi) a set of LAMP primers including four primers including nucleic acid sequences having at least 99% sequence identity to the sequences of SEQ ID NO:31, SEQ ID NO:32, SEQ ID NO:33 or SEQ ID NO:34.

In embodiments, the set of loop-mediated isothermal amplification (LAMP) primers includes: (i) a set of LAMP primers including four primers including nucleic acid sequences having at least 99% sequence identity to the sequences of SEQ ID NO:1, SEQ ID NO:2, SEQ ID NO:3 or SEQ ID NO:4; (ii) a set of LAMP primers including four primers including nucleic acid sequences having at least 99% sequence identity to the sequences of SEQ ID NO:7, SEQ ID NO:8, SEQ ID NO:9 or SEQ ID NO:10; (iii) a set of LAMP primers including four primers including nucleic acid sequences having at least 99% sequence identity to the sequences of SEQ ID NO:13, SEQ ID NO:14, SEQ ID NO:15 or SEQ ID NO:16; (iv) a set of LAMP primers including four primers including nucleic acid sequences having at least 99% sequence identity to the sequences of SEQ ID NO:19, SEQ ID NO:20, SEQ ID NO:21 or SEQ ID NO:22; (v) a set of LAMP primers including four primers including nucleic acid sequences having at least 99% sequence identity to the sequences of SEQ ID NO:25, SEQ ID NO:26, SEQ ID NO:27 or SEQ ID NO:28; or (vi) a set of LAMP primers including four primers including nucleic acid sequences having at least 99% sequence identity to the sequences of SEQ ID NO:31, SEQ ID NO:32, SEQ ID NO:37 or SEQ ID NO:34.

In embodiments, the set of loop-mediated isothermal amplification (LAMP) primers includes: (i) a set of LAMP primers including four primers including nucleic acid sequences having 99% sequence identity to the sequences of SEQ ID NO:1, SEQ ID NO:2, SEQ ID NO:3 or SEQ ID NO:4; (ii) a set of LAMP primers including four primers including nucleic acid sequences having 99% sequence identity to the sequences of SEQ ID NO:7, SEQ ID NO:8, SEQ ID NO:9 or SEQ ID NO:10; (iii) a set of LAMP primers including four primers including nucleic acid sequences having 99% sequence identity to the sequences of SEQ ID NO:13, SEQ ID NO:14, SEQ ID NO:15 or SEQ ID NO:16; (iv) a set of LAMP primers including four primers including nucleic acid sequences having 99% sequence identity to the sequences of SEQ ID NO:19, SEQ ID NO:20, SEQ ID NO:21 or SEQ ID NO:22; (v) a set of LAMP primers including four primers including nucleic acid sequences having 99% sequence identity to the sequences of SEQ ID NO:25, SEQ ID NO:26, SEQ ID NO:27 or SEQ ID NO:28; or (vi) a set of LAMP primers including four primers including nucleic acid sequences having 99% sequence identity to the sequences of SEQ ID NO:31, SEQ ID NO:32, SEQ ID NO:33 or SEQ ID NO:34.

In embodiments, the set of loop-mediated isothermal amplification (LAMP) primers includes: (i) a set of LAMP primers including four primers including nucleic acid sequences having 99% sequence identity to the sequences of SEQ ID NO:1, SEQ ID NO:2, SEQ ID NO:3 or SEQ ID NO:4; (ii) a set of LAMP primers including four primers including nucleic acid sequences having 99% sequence identity to the sequences of SEQ ID NO:7, SEQ ID NO:8, SEQ ID NO:9 or SEQ ID NO:10; (iii) a set of LAMP primers including four primers including nucleic acid sequences having 99% sequence identity to the sequences of SEQ ID NO:13, SEQ ID NO:14, SEQ ID NO:15 or SEQ ID NO:16; (iv) a set of LAMP primers including four primers including nucleic acid sequences having 99% sequence identity to the sequences of SEQ ID NO:19, SEQ ID NO:20, SEQ ID NO:21 or SEQ ID NO:22; (v) a set of LAMP primers including four primers including nucleic acid sequences having 99% sequence identity to the sequences of SEQ ID NO:25, SEQ ID NO:26, SEQ ID NO:27 or SEQ ID NO:28; or (vi) a set of LAMP primers including four primers including nucleic acid sequences having 99% sequence identity to the sequences of SEQ ID NO:31, SEQ ID NO:32, SEQ ID NO:37 or SEQ ID NO:34.

In embodiments, the set of loop-mediated isothermal amplification (LAMP) primers includes: (i) a set of LAMP primers including four primers including nucleic acid sequences having 100% sequence identity to the sequences of SEQ ID NO:1, SEQ ID NO:2, SEQ ID NO:3 or SEQ ID NO:4; (ii) a set of LAMP primers including four primers including nucleic acid sequences having 100% sequence identity to the sequences of SEQ ID NO:7, SEQ ID NO:8, SEQ ID NO:9 or SEQ ID NO:10; (iii) a set of LAMP primers including four primers including nucleic acid sequences having 100% sequence identity to the sequences of SEQ ID NO:13, SEQ ID NO:14, SEQ ID NO:15 or SEQ ID NO:16; (iv) a set of LAMP primers including four primers including nucleic acid sequences having 100% sequence identity to the sequences of SEQ ID NO:19, SEQ ID NO:20, SEQ ID NO:21 or SEQ ID NO:22; (v) a set of LAMP primers including four primers including nucleic acid sequences having 100% sequence identity to the sequences of SEQ ID NO:25, SEQ ID NO:26, SEQ ID NO:27 or SEQ ID NO:28; or (vi) a set of LAMP primers including four primers including nucleic acid sequences having 100% sequence identity to the sequences of SEQ ID NO:31, SEQ ID NO:32, SEQ ID NO:33 or SEQ ID NO:34.

In embodiments, the set of loop-mediated isothermal amplification (LAMP) primers includes: (i) a set of LAMP primers including four primers including nucleic acid sequences having 100% sequence identity to the sequences of SEQ ID NO:1, SEQ ID NO:2, SEQ ID NO:3 or SEQ ID NO:4; (ii) a set of LAMP primers including four primers including nucleic acid sequences having 100% sequence identity to the sequences of SEQ ID NO:7, SEQ ID NO:8, SEQ ID NO:9 or SEQ ID NO:10; (iii) a set of LAMP primers including four primers including nucleic acid sequences having 100% sequence identity to the sequences of SEQ ID NO:13, SEQ ID NO:14, SEQ ID NO:15 or SEQ ID NO:16; (iv) a set of LAMP primers including four primers including nucleic acid sequences having 100% sequence identity to the sequences of SEQ ID NO:19, SEQ ID NO:20, SEQ ID NO:21 or SEQ ID NO:22; (v) a set of LAMP primers including four primers including nucleic acid sequences having 100% sequence identity to the sequences of SEQ ID NO:25, SEQ ID NO:26, SEQ ID NO:27 or SEQ ID NO:28; or (vi) a set of LAMP primers including four primers including nucleic acid sequences having 100% sequence identity to the sequences of SEQ ID NO:31, SEQ ID NO:32, SEQ ID NO:37 or SEQ ID NO:34.

In an aspect is provided a kit including a set of loop-mediated isothermal amplification (LAMP) primers including: (i) a set of LAMP primers including four primers including nucleic acid sequences having at least 90% sequence identity to the sequences of SEQ ID NO:1, SEQ ID NO:2, SEQ ID NO:3 and SEQ ID NO:4; (ii) a set of LAMP primers including four primers including nucleic acid sequences having at least 90% sequence identity to the sequences of SEQ ID NO:7, SEQ ID NO:8, SEQ ID NO:9 and SEQ ID NO:10; (iii) a set of LAMP primers including four primers including nucleic acid sequences having at least 90% sequence identity to the sequences of SEQ ID NO:13, SEQ ID NO:14, SEQ ID NO:15 and SEQ ID NO:16; (iv) a set of LAMP primers including four primers including nucleic acid sequences having at least 90% sequence identity to the sequences of SEQ ID NO:19, SEQ ID NO:20, SEQ ID NO:21 and SEQ ID NO:22; (v) a set of LAMP primers including four primers including nucleic acid sequences having at least 90% sequence identity to the sequences of SEQ ID NO:25, SEQ ID NO:26, SEQ ID NO:27 and SEQ ID NO:28; or (vi) a set of LAMP primers including four primers including nucleic acid sequences having at least 90% sequence identity to the sequences of SEQ ID NO:31, SEQ ID NO:32, SEQ ID NO:33 and SEQ ID NO:34.

In an aspect is provided a kit including a set of loop-mediated isothermal amplification (LAMP) primers including: (i) a set of LAMP primers including four primers including nucleic acid sequences having at least 90% sequence identity to the sequences of SEQ ID NO:1, SEQ ID NO:2, SEQ ID NO:3 and SEQ ID NO:4; (ii) a set of LAMP primers including four primers including nucleic acid sequences having at least 90% sequence identity to the sequences of SEQ ID NO:7, SEQ ID NO:8, SEQ ID NO:9 and SEQ ID NO:10; (iii) a set of LAMP primers including four primers including nucleic acid sequences having at least 90% sequence identity to the sequences of SEQ ID NO:13, SEQ ID NO:14, SEQ ID NO:15 and SEQ ID NO:16; (iv) a set of LAMP primers including four primers including nucleic acid sequences having at least 90% sequence identity to the sequences of SEQ ID NO:19, SEQ ID NO:20, SEQ ID NO:21 and SEQ ID NO:22; (v) a set of LAMP primers including four primers including nucleic acid sequences having at least 90% sequence identity to the sequences of SEQ ID NO:25, SEQ ID NO:26, SEQ ID NO:27 and SEQ ID NO:28; or (vi) a set of LAMP primers including four primers including nucleic acid sequences having at least 90% sequence identity to the sequences of SEQ ID NO:31, SEQ ID NO:32, SEQ ID NO:37 and SEQ ID NO:34.

In embodiments, the set of LAMP primers in (i) further includes a set of LAMP primers including two primers including nucleic acid sequences having at least 90% sequence identity to the sequences of SEQ ID NO:5 or SEQ ID NO:6. In embodiments, the set of LAMP primers in (i) further includes a set of LAMP primers including two primers including nucleic acid sequences having at least 95% sequence identity to the sequences of SEQ ID NO:5 or SEQ ID NO:6. In embodiments, the set of LAMP primers in (i) further includes a set of LAMP primers including two primers including nucleic acid sequences having at least 98% sequence identity to the sequences of SEQ ID NO:5 or SEQ ID NO:6. In embodiments, the set of LAMP primers in (i) further includes a set of LAMP primers including two primers including nucleic acid sequences having at least 99% sequence identity to the sequences of SEQ ID NO:5 or SEQ ID NO:6.

In embodiments, the set of LAMP primers in (i) further includes a set of LAMP primers including two primers including nucleic acid sequences having 90% sequence identity to the sequences of SEQ ID NO:5 or SEQ ID NO:6. In embodiments, the set of LAMP primers in (i) further includes a set of LAMP primers including two primers including nucleic acid sequences having 95% sequence identity to the sequences of SEQ ID NO:5 or SEQ ID NO:6. In embodiments, the set of LAMP primers in (i) further includes a set of LAMP primers including two primers including nucleic acid sequences having 98% sequence identity to the sequences of SEQ ID NO:5 or SEQ ID NO:6. In embodiments, the set of LAMP primers in (i) further includes a set of LAMP primers including two primers including nucleic acid sequences having 99% sequence identity to the sequences of SEQ ID NO:5 or SEQ ID NO:6. In embodiments, the set of LAMP primers in (i) further includes a set of LAMP primers including two primers including nucleic acid sequences having 100% sequence identity to the sequences of SEQ ID NO:5 or SEQ ID NO:6.

In embodiments, the set of LAMP primers in (ii) further includes a set of LAMP primers including two primers including nucleic acid sequences having at least 90% sequence identity to the sequences of SEQ ID NO:11 or SEQ ID NO:12. In embodiments, the set of LAMP primers in (ii) further includes a set of LAMP primers including two primers including nucleic acid sequences having at least 95% sequence identity to the sequences of SEQ ID NO:11 or SEQ ID NO:12. In embodiments, the set of LAMP primers in (ii) further includes a set of LAMP primers including two primers including nucleic acid sequences having at least 98% sequence identity to the sequences of SEQ ID NO:11 or SEQ ID NO:12. In embodiments, the set of LAMP primers in (ii) further includes a set of LAMP primers including two primers including nucleic acid sequences having at least 99% sequence identity to the sequences of SEQ ID NO:11 or SEQ ID NO:12.

In embodiments, the set of LAMP primers in (ii) further includes a set of LAMP primers including two primers including nucleic acid sequences having 90% sequence identity to the sequences of SEQ ID NO:11 or SEQ ID NO:12. In embodiments, the set of LAMP primers in (ii) further includes a set of LAMP primers including two primers including nucleic acid sequences having 95% sequence identity to the sequences of SEQ ID NO:11 or SEQ ID NO:12. In embodiments, the set of LAMP primers in (ii) further includes a set of LAMP primers including two primers including nucleic acid sequences having 98% sequence identity to the sequences of SEQ ID NO:11 or SEQ ID NO:12. In embodiments, the set of LAMP primers in (ii) further includes a set of LAMP primers including two primers including nucleic acid sequences having 99% sequence identity to the sequences of SEQ ID NO:11 or SEQ ID NO:12. In embodiments, the set of LAMP primers in (ii) further includes a set of LAMP primers including two primers including nucleic acid sequences having 100% sequence identity to the sequences of SEQ ID NO:11 or SEQ ID NO:12.

In embodiments, the set of LAMP primers in (iii) further includes a set of LAMP primers including two primers including nucleic acid sequences having at least 90% sequence identity to the sequences of SEQ ID NO:17 or SEQ ID NO:18. In embodiments, the set of LAMP primers in (iii) further includes a set of LAMP primers including two primers including nucleic acid sequences having at least 95% sequence identity to the sequences of SEQ ID NO:17 or SEQ ID NO:18. In embodiments, the set of LAMP primers in (iii) further includes a set of LAMP primers including two primers including nucleic acid sequences having at least 98% sequence identity to the sequences of SEQ ID NO:17 or SEQ ID NO:18.

In embodiments, the set of LAMP primers in (iii) further includes a set of LAMP primers including two primers including nucleic acid sequences having at least 99% sequence identity to the sequences of SEQ ID NO:17 or SEQ ID NO:18.

In embodiments, the set of LAMP primers in (iii) further includes a set of LAMP primers including two primers including nucleic acid sequences having 90% sequence identity to the sequences of SEQ ID NO:17 or SEQ ID NO:18. In embodiments, the set of LAMP primers in (iii) further includes a set of LAMP primers including two primers including nucleic acid sequences having 95% sequence identity to the sequences of SEQ ID NO:17 or SEQ ID NO:18. In embodiments, the set of LAMP primers in (iii) further includes a set of LAMP primers including two primers including nucleic acid sequences having 98% sequence identity to the sequences of SEQ ID NO:17 or SEQ ID NO:18. In embodiments, the set of LAMP primers in (iii) further includes a set of LAMP primers including two primers including nucleic acid sequences having 99% sequence identity to the sequences of SEQ ID NO:17 or SEQ ID NO:18. In embodiments, the set of LAMP primers in (iii) further includes a set of LAMP primers including two primers including nucleic acid sequences having 100% sequence identity to the sequences of SEQ ID NO:17 or SEQ ID NO:18.

In embodiments, the set of LAMP primers in (iv) further includes a set of LAMP primers including two primers including nucleic acid sequences having at least 90% sequence identity to the sequences of SEQ ID NO:23 or SEQ ID NO:24. In embodiments, the set of LAMP primers in (iv) further includes a set of LAMP primers including two primers including nucleic acid sequences having at least 95% sequence identity to the sequences of SEQ ID NO:23 or SEQ ID NO:24. In embodiments, the set of LAMP primers in (iv) further includes a set of LAMP primers including two primers including nucleic acid sequences having at least 98% sequence identity to the sequences of SEQ ID NO:23 or SEQ ID NO:24. In embodiments, the set of LAMP primers in (iv) further includes a set of LAMP primers including two primers including nucleic acid sequences having at least 99% sequence identity to the sequences of SEQ ID NO:23 or SEQ ID NO:24.

In embodiments, the set of LAMP primers in (iv) further includes a set of LAMP primers including two primers including nucleic acid sequences having 90% sequence identity to the sequences of SEQ ID NO:23 or SEQ ID NO:24. In embodiments, the set of LAMP primers in (iv) further includes a set of LAMP primers including two primers including nucleic acid sequences having 95% sequence identity to the sequences of SEQ ID NO:23 or SEQ ID NO:24. In embodiments, the set of LAMP primers in (iv) further includes a set of LAMP primers including two primers including nucleic acid sequences having 98% sequence identity to the sequences of SEQ ID NO:23 or SEQ ID NO:24. In embodiments, the set of LAMP primers in (iv) further includes a set of LAMP primers including two primers including nucleic acid sequences having 99% sequence identity to the sequences of SEQ ID NO:23 or SEQ ID NO:24. In embodiments, the set of LAMP primers in (iv) further includes a set of LAMP primers including two primers including nucleic acid sequences having 100% sequence identity to the sequences of SEQ ID NO:23 or SEQ ID NO:24.

In embodiments, the set of LAMP primers in (iv) further includes a set of LAMP

primers including two primers including nucleic acid sequences having at least 90% sequence identity to the sequences of SEQ ID NO:29 or SEQ ID NO:30. In embodiments, the set of LAMP primers in (iv) further includes a set of LAMP primers including two primers including nucleic acid sequences having at least 95% sequence identity to the sequences of SEQ ID NO:29 or SEQ ID NO:30. In embodiments, the set of LAMP primers in (iv) further includes a set of LAMP primers including two primers including nucleic acid sequences having at least 98% sequence identity to the sequences of SEQ ID NO:29 or SEQ ID NO:30.

In embodiments, the set of LAMP primers in (iv) further includes a set of LAMP primers including two primers including nucleic acid sequences having at least 99% sequence identity to the sequences of SEQ ID NO:29 or SEQ ID NO:30.

In embodiments, the set of LAMP primers in (iv) further includes a set of LAMP primers including two primers including nucleic acid sequences having 90% sequence identity to the sequences of SEQ ID NO:29 or SEQ ID NO:30. In embodiments, the set of LAMP primers in (iv) further includes a set of LAMP primers including two primers including nucleic acid sequences having 95% sequence identity to the sequences of SEQ ID NO:29 or SEQ ID NO:30. In embodiments, the set of LAMP primers in (iv) further includes a set of LAMP primers including two primers including nucleic acid sequences having 98% sequence identity to the sequences of SEQ ID NO:29 or SEQ ID NO:30. In embodiments, the set of LAMP primers in (iv) further includes a set of LAMP primers including two primers including nucleic acid sequences having 99% sequence identity to the sequences of SEQ ID NO:29 or SEQ ID NO:30. In embodiments, the set of LAMP primers in (iv) further includes a set of LAMP primers including two primers including nucleic acid sequences having 100% sequence identity to the sequences of SEQ ID NO:29 or SEQ ID NO:30.

In embodiments, the set of LAMP primers in (iv) further includes a set of LAMP primers including two primers including nucleic acid sequences having at least 90% sequence identity to the sequences of SEQ ID NO:35 or SEQ ID NO:36. In embodiments, the set of LAMP primers in (iv) further includes a set of LAMP primers including two primers including nucleic acid sequences having at least 95% sequence identity to the sequences of SEQ ID NO:35 or SEQ ID NO:36. In embodiments, the set of LAMP primers in (iv) further includes a set of LAMP primers including two primers including nucleic acid sequences having at least 98% sequence identity to the sequences of SEQ ID NO:35 or SEQ ID NO:36. In embodiments, the set of LAMP primers in (iv) further includes a set of LAMP primers including two primers including nucleic acid sequences having at least 99% sequence identity to the sequences of SEQ ID NO:35 or SEQ ID NO:36.

In embodiments, the set of LAMP primers in (iv) further includes a set of LAMP primers including two primers including nucleic acid sequences having 90% sequence identity to the sequences of SEQ ID NO:35 or SEQ ID NO:36. In embodiments, the set of LAMP primers in (iv) further includes a set of LAMP primers including two primers including nucleic acid sequences having 95% sequence identity to the sequences of SEQ ID NO:35 or SEQ ID NO:36. In embodiments, the set of LAMP primers in (iv) further includes a set of LAMP primers including two primers including nucleic acid sequences having 98% sequence identity to the sequences of SEQ ID NO:35 or SEQ ID NO:36. In embodiments, the set of LAMP primers in (iv) further includes a set of LAMP primers including two primers including nucleic acid sequences having 99% sequence identity to the sequences of SEQ ID NO:35 or SEQ ID NO:36. In embodiments, the set of LAMP primers in (iv) further includes a set of LAMP primers including two primers including nucleic acid sequences having 100% sequence identity to the sequences of SEQ ID NO:35 or SEQ ID NO:36.

In an aspect is provided a kit including a set of loop-mediated isothermal amplification (LAMP) primers including a set of LAMP primers including four primers including nucleic acid sequences having at least 90% sequence identity to the sequences of SEQ ID NO:1, SEQ ID NO:2, SEQ ID NO:3 or SEQ ID NO:4. In embodiments, the set of LAMP primers further includes a set of LAMP primers including two primers including nucleic acid sequences having at least 90% sequence identity to the sequences of SEQ ID NO:5 or SEQ ID NO:6. In one embodiment, the set of LAMP primers includes a set of LAMP primers including six primers including nucleic acid sequences having at least 90% sequence identity to the sequences of SEQ ID NO:1, SEQ ID NO:2, SEQ ID NO:3, SEQ ID NO:4, SEQ ID NO:5 or SEQ ID NO:6. In embodiments, the at least 90% sequence identity is 91%, 92%, 93%, 94%, 95%, 96%, 97%, 98%, 99% or 100%.

In an aspect is provided a kit including a set of loop-mediated isothermal amplification (LAMP) primers including a set of LAMP primers including four primers including nucleic acid sequences having at least 90% sequence identity to the sequences of SEQ ID NO:1, SEQ ID NO:2, SEQ ID NO:3 and SEQ ID NO:4. In embodiments, the set of LAMP primers further includes a set of LAMP primers including two primers including nucleic acid sequences having at least 90% sequence identity to the sequences of SEQ ID NO:5 and SEQ ID NO:6. In one embodiment, the set of LAMP primers includes a set of LAMP primers including six primers including nucleic acid sequences having at least 90% sequence identity to the sequences of SEQ ID NO:1, SEQ ID NO:2, SEQ ID NO:3, SEQ ID NO:4, SEQ ID NO:5 and SEQ ID NO:6. In embodiments, the at least 90% sequence identity is 91%, 92%, 93%, 94%, 95%, 96%, 97%, 98%, 99% or 100%. In one embodiment, the set of LAMP primers includes a set of LAMP primers including six primers with the sequences of SEQ ID NO:1, SEQ ID NO:2, SEQ ID NO:3, SEQ ID NO:4, SEQ ID NO:5 and SEQ ID NO:6.

In an aspect is provided a kit including a set of loop-mediated isothermal amplification (LAMP) primers including a set of LAMP primers including four primers including nucleic acid sequences having at least 90% sequence identity to the sequences of SEQ ID NO:7, SEQ ID NO:8, SEQ ID NO:9 or SEQ ID NO:10. In embodiments, the set of LAMP primers further includes a set of LAMP primers including two primers including nucleic acid sequences having at least 90% sequence identity to the sequences of SEQ ID NO:11 or SEQ ID NO:12. In one embodiment, the set of LAMP primers includes a set of LAMP primers including six primers including nucleic acid sequences having at least 90% sequence identity to the sequences of SEQ ID NO:7, SEQ ID NO:8, SEQ ID NO:9, SEQ ID NO:10, SEQ ID NO:11 or SEQ ID NO:12. In embodiments, the at least 90% sequence identity is 91%, 92%, 93%, 94%, 95%, 96%, 97%, 98%, 99% or 100%.

In an aspect is provided a kit including a set of loop-mediated isothermal amplification (LAMP) primers including a set of LAMP primers including four primers including nucleic acid sequences having at least 90% sequence identity to the sequences of SEQ ID NO:7, SEQ ID NO:8, SEQ ID NO:9 and SEQ ID NO:10. In embodiments, the set of LAMP primers further includes a set of LAMP primers including two primers including nucleic acid sequences having at least 90% sequence identity to the sequences of SEQ ID NO:11 and SEQ ID NO:12. In one embodiment, the set of LAMP primers includes a set of LAMP primers including six primers including nucleic acid sequences having at least 90% sequence identity to the sequences of SEQ ID NO:7, SEQ ID NO:8, SEQ ID NO:9, SEQ ID NO:10, SEQ ID NO:11 and SEQ ID NO:12. In embodiments, the at least 90% sequence identity is 91%, 92%, 93%, 94%, 95%, 96%, 97%, 98%, 99% or 100%. In one embodiment, the set of LAMP primers includes a set of LAMP primers including six primers with the sequences of SEQ ID NO:7, SEQ ID NO:8, SEQ ID NO:9, SEQ ID NO:10, SEQ ID NO:11 and SEQ ID NO:12.

In an aspect is provided a kit including a set of loop-mediated isothermal amplification (LAMP) primers including a set of LAMP primers including four primers including nucleic acid sequences having at least 90% sequence identity to the sequences of SEQ ID NO:13, SEQ ID NO:14, SEQ ID NO:15 or SEQ ID NO:16. In embodiments, the set of LAMP primers further includes a set of LAMP primers including two primers including nucleic acid sequences having at least 90% sequence identity to the sequences of SEQ ID NO:17 or SEQ ID NO:18. In one embodiment, the set of LAMP primers includes a set of LAMP primers including six primers including nucleic acid sequences having at least 90% sequence identity to the sequences of SEQ ID NO:13, SEQ ID NO:14, SEQ ID NO:15, SEQ ID NO:16, SEQ ID NO:17 or SEQ ID NO:18. In embodiments, the at least 90% sequence identity is 91%, 92%, 93%, 94%, 95%, 96%, 97%, 98%, 99% or 100%.

In an aspect is provided a kit including a set of loop-mediated isothermal amplification (LAMP) primers including a set of LAMP primers including four primers including nucleic acid sequences having at least 90% sequence identity to the sequences of SEQ ID NO:13, SEQ ID NO:14, SEQ ID NO:15 and SEQ ID NO:16. In embodiments, the set of LAMP primers further includes a set of LAMP primers including two primers including nucleic acid sequences having at least 90% sequence identity to the sequences of SEQ ID NO:17 and SEQ ID NO:18. In one embodiment, the set of LAMP primers includes a set of LAMP primers including six primers including nucleic acid sequences having at least 90% sequence identity to the sequences of SEQ ID NO:13, SEQ ID NO:14, SEQ ID NO:15, SEQ ID NO:16, SEQ ID NO:17 and SEQ ID NO:18. In embodiments, the at least 90% sequence identity is 91%, 92%, 93%, 94%, 95%, 96%, 97%, 98%, 99% or 100%. In one embodiment, the set of LAMP primers includes a set of LAMP primers including six primers with the sequences of SEQ ID NO:13, SEQ ID NO:14, SEQ ID NO:15, SEQ ID NO:16, SEQ ID NO:17 and SEQ ID NO:18.

In an aspect is provided a kit including a set of loop-mediated isothermal amplification (LAMP) primers including a set of LAMP primers including four primers including nucleic acid sequences having at least 90% sequence identity to the sequences of SEQ ID NO:19, SEQ ID NO:20, SEQ ID NO:21 or SEQ ID NO:22. In embodiments, the set of LAMP primers further includes a set of LAMP primers including two primers including nucleic acid sequences having at least 90% sequence identity to the sequences of SEQ ID NO:23 or SEQ ID NO:24. In one embodiment, the set of LAMP primers includes a set of LAMP primers including six primers including nucleic acid sequences having at least 90% sequence identity to the sequences of SEQ ID NO:19, SEQ ID NO:20, SEQ ID NO:21, SEQ ID NO:22, SEQ ID NO:23 or SEQ ID NO:24. In embodiments, the at least 90% sequence identity is 91%, 92%, 93%, 94%, 95%, 96%, 97%, 98%, 99% or 100%.

In an aspect is provided a kit including a set of loop-mediated isothermal amplification (LAMP) primers including a set of LAMP primers including four primers including nucleic acid sequences having at least 90% sequence identity to the sequences of SEQ ID NO:19, SEQ ID NO:20, SEQ ID NO:21 and SEQ ID NO:22. In embodiments, the set of LAMP primers further includes a set of LAMP primers including two primers including nucleic acid sequences having at least 90% sequence identity to the sequences of SEQ ID NO:23 and SEQ ID NO:24. In one embodiment, the set of LAMP primers includes a set of LAMP primers including six primers including nucleic acid sequences having at least 90% sequence identity to the sequences of SEQ ID NO:19, SEQ ID NO:20, SEQ ID NO:21, SEQ ID NO:22, SEQ ID NO:23 and SEQ ID NO:24. In embodiments, the at least 90% sequence identity is 91%, 92%, 93%, 94%, 95%, 96%, 97%, 98%, 99% or 100%. In one embodiment, the set of LAMP primers includes a set of LAMP primers including six primers with the sequences of SEQ ID NO:19, SEQ ID NO:20, SEQ ID NO:21, SEQ ID NO:22, SEQ ID NO:23 and SEQ ID NO:24.

In an aspect is provided a kit including a set of loop-mediated isothermal amplification (LAMP) primers including a set of LAMP primers including four primers including nucleic acid sequences having at least 90% sequence identity to the sequences of SEQ ID NO:25, SEQ ID NO:26, SEQ ID NO:27 or SEQ ID NO:28. In embodiments, the set of LAMP primers further includes a set of LAMP primers including two primers including nucleic acid sequences having at least 90% sequence identity to the sequences of SEQ ID NO:29 or SEQ ID NO:30. In one embodiment, the set of LAMP primers includes a set of LAMP primers including six primers including nucleic acid sequences having at least 90% sequence identity to the sequences of SEQ ID NO:25, SEQ ID NO:26, SEQ ID NO:27, SEQ ID NO:28, SEQ ID NO:29 or SEQ ID NO:30. In embodiments, the at least 90% sequence identity is 91%, 92%, 93%, 94%, 95%, 96%, 97%, 98%, 99% or 100%.

In an aspect is provided a kit including a set of loop-mediated isothermal amplification (LAMP) primers including a set of LAMP primers including four primers including nucleic acid sequences having at least 90% sequence identity to the sequences of SEQ ID NO:25, SEQ ID NO:26, SEQ ID NO:27 and SEQ ID NO:28. In embodiments, the set of LAMP primers further includes a set of LAMP primers including two primers including nucleic acid sequences having at least 90% sequence identity to the sequences of SEQ ID NO:29 and SEQ ID NO:30. In one embodiment, the set of LAMP primers includes a set of LAMP primers including six primers including nucleic acid sequences having at least 90% sequence identity to the sequences of SEQ ID NO:25, SEQ ID NO:26, SEQ ID NO:27, SEQ ID NO:28, SEQ ID NO:29 and SEQ ID NO:30. In embodiments, the at least 90% sequence identity is 91%, 92%, 93%, 94%, 95%, 96%, 97%, 98%, 99% or 100%. In one embodiment, the set of LAMP primers includes a set of LAMP primers including six primers with the sequences of SEQ ID NO:25, SEQ ID NO:26, SEQ ID NO:27, SEQ ID NO:28, SEQ ID NO:29 and SEQ ID NO:30.

In an aspect is provided a kit including a set of loop-mediated isothermal amplification (LAMP) primers including a set of LAMP primers including four primers including nucleic acid sequences having at least 90% sequence identity to the sequences of SEQ ID NO:31, SEQ ID NO:32, SEQ ID NO:33 or SEQ ID NO:34. In embodiments, the set of LAMP primers further includes a set of LAMP primers including two primers including nucleic acid sequences having at least 90% sequence identity to the sequences of SEQ ID NO:35 or SEQ ID NO:36. In one embodiment, the set of LAMP primers includes a set of LAMP primers including six primers including nucleic acid sequences having at least 90% sequence identity to the sequences of SEQ ID NO:31, SEQ ID NO:32, SEQ ID NO:33, SEQ ID NO:34, SEQ ID NO:35 or SEQ ID NO:36. In embodiments, the at least 90% sequence identity is 91%, 92%, 93%, 94%, 95%, 96%, 97%, 98%, 99% or 100%.

In an aspect is provided a kit including a set of loop-mediated isothermal amplification (LAMP) primers including a set of LAMP primers including four primers including nucleic acid sequences having at least 90% sequence identity to the sequences of SEQ ID NO:31, SEQ ID NO:32, SEQ ID NO:37 or SEQ ID NO:34. In embodiments, the set of LAMP primers further includes a set of LAMP primers including two primers including nucleic acid sequences having at least 90% sequence identity to the sequences of SEQ ID NO:35 or SEQ ID NO:36. In one embodiment, the set of LAMP primers includes a set of LAMP primers including six primers including nucleic acid sequences having at least 90% sequence identity to the sequences of SEQ ID NO:31, SEQ ID NO:32, SEQ ID NO:37, SEQ ID NO:34, SEQ ID NO:35 or SEQ ID NO:36. In embodiments, the at least 90% sequence identity is 91%, 92%, 93%, 94%, 95%, 96%, 97%, 98%, 99% or 100%.

In an aspect is provided a kit including a set of loop-mediated isothermal amplification (LAMP) primers including a set of LAMP primers including four primers including nucleic acid sequences having at least 90% sequence identity to the sequences of SEQ ID NO:31, SEQ ID NO:32, SEQ ID NO:33 and SEQ ID NO:34. In embodiments, the set of LAMP primers further includes a set of LAMP primers including two primers including nucleic acid sequences having at least 90% sequence identity to the sequences of SEQ ID NO:35 and SEQ ID NO:36. In one embodiment, the set of LAMP primers includes a set of LAMP primers including six primers including nucleic acid sequences having at least 90% sequence identity to the sequences of SEQ ID NO:31, SEQ ID NO:32, SEQ ID NO:33, SEQ ID NO:34, SEQ ID NO:35 and SEQ ID NO:36. In embodiments, the at least 90% sequence identity is 91%, 92%, 93%, 94%, 95%, 96%, 97%, 98%, 99% or 100%. In one embodiment, the set of LAMP primers includes a set of LAMP primers including six primers with the sequences of SEQ ID NO:31, SEQ ID NO:32, SEQ ID NO:33, SEQ ID NO:34, SEQ ID NO:35 and SEQ ID NO:36.

In an aspect is provided a kit including a set of loop-mediated isothermal amplification (LAMP) primers including a set of LAMP primers including four primers including nucleic acid sequences having at least 90% sequence identity to the sequences of SEQ ID NO:31, SEQ ID NO:32, SEQ ID NO:37 and SEQ ID NO:34. In embodiments, the set of LAMP primers further includes a set of LAMP primers including two primers including nucleic acid sequences having at least 90% sequence identity to the sequences of SEQ ID NO:35 and SEQ ID NO:36. In one embodiment, the set of LAMP primers includes a set of LAMP primers including six primers including nucleic acid sequences having at least 90% sequence identity to the sequences of SEQ ID NO:31, SEQ ID NO:32, SEQ ID NO:37, SEQ ID NO:34, SEQ ID NO:35 and SEQ ID NO:36. In embodiments, the at least 90% sequence identity is 91%, 92%, 93%, 94%, 95%, 96%, 97%, 98%, 99% or 100%. In one embodiment, the set of LAMP primers includes a set of LAMP primers including six primers with the sequences of SEQ ID NO:31, SEQ ID NO:32, SEQ ID NO:37, SEQ ID NO:34, SEQ ID NO:35 and SEQ ID NO:36.

In embodiments, the set of LAMP primers is specific for an SARS-CoV-2 nucleic acid. In embodiments, the set of LAMP primers is specific for an SARS-CoV-2 variant (e.g., delta) nucleic acid. In embodiments, the SARS-CoV-2 nucleic acid encodes a 5′-UTR/ORF1ab, a spike protein, nucleocapsid gene or a functional fragment thereof. In embodiments, the SARS-CoV-2 nucleic acid encodes a 5′-UTR/ORF1ab or a functional fragment thereof. In embodiments, the SARS-CoV-2 nucleic acid encodes a spike protein or a functional fragment thereof. In embodiments, the SARS-CoV-2 nucleic acid encodes a nucleocapsid gene or a functional fragment thereof.

In embodiments, the kit further includes a strand-displacement polymerase. In embodiments, the strand-displacement polymerase is a Bst polymerase.

In embodiments, the set of LAMP primers is in a container. In embodiments, each of the sets of LAMP primers is in a separate container. In embodiments, each primer of the set of LAMP primers is in a separate container. In embodiments, the container is in a detection device.

### Methods of Detection

In another aspect is provided a method of detecting a SARS-CoV-2 nucleic acid in a sample, the method including: (a) contacting a sample with a set of loop-mediated isothermal amplification (LAMP) primers under conditions sufficient to amplify a SARS-CoV-2 nucleic acid, wherein the set of LAMP primers includes: (i) a set of LAMP primers including four primers including nucleic acid sequences having at least 90% sequence identity to the sequences of SEQ ID NO:1, SEQ ID NO:2, SEQ ID NO:3 or SEQ ID NO:4; (ii) a set of LAMP primers including four primers including nucleic acid sequences having at least 90% sequence identity to the sequences of SEQ ID NO:7, SEQ ID NO:8, SEQ ID NO:9 or SEQ ID NO:10; (iii) a set of LAMP primers including four primers including nucleic acid sequences having at least 90% sequence identity to the sequences of SEQ ID NO:13, SEQ ID NO:14, SEQ ID NO:15 or SEQ ID NO:16; (iv) a set of LAMP primers including four primers including nucleic acid sequences having at least 90% sequence identity to the sequences of SEQ ID NO:19, SEQ ID NO:20, SEQ ID NO:21 or SEQ ID NO:22; (v) a set of LAMP primers including four primers including nucleic acid sequences having at least 90% sequence identity to the sequences of SEQ ID NO:25, SEQ ID NO:26, SEQ ID NO:27 or SEQ ID NO:28; or (vi) a set of LAMP primers including four primers including nucleic acid sequences having at least 90% sequence identity to the sequences of SEQ ID NO:31, SEQ ID NO:32, SEQ ID NO:33 or SEQ ID NO:34; thereby forming a SARS-CoV-2 amplification product; and (b) detecting the SARS-CoV-2 amplification product, thereby detecting the SARS-CoV-2 nucleic acid in the sample.

In another aspect is provided a method of detecting a SARS-CoV-2 nucleic acid in a sample, the method including: (a) contacting a sample with a set of loop-mediated isothermal amplification (LAMP) primers under conditions sufficient to amplify a SARS-CoV-2 nucleic acid, wherein the set of LAMP primers includes: (i) a set of LAMP primers including four primers including nucleic acid sequences having at least 90% sequence identity to the sequences of SEQ ID NO:1, SEQ ID NO:2, SEQ ID NO:3 or SEQ ID NO:4; (ii) a set of LAMP primers including four primers including nucleic acid sequences having at least 90% sequence identity to the sequences of SEQ ID NO:7, SEQ ID NO:8, SEQ ID NO:9 or SEQ ID NO:10; (iii) a set of LAMP primers including four primers including nucleic acid sequences having at least 90% sequence identity to the sequences of SEQ ID NO:13, SEQ ID NO:14, SEQ ID NO:15 or SEQ ID NO:16; (iv) a set of LAMP primers including four primers including nucleic acid sequences having at least 90% sequence identity to the sequences of SEQ ID NO:19, SEQ ID NO:20, SEQ ID NO:21 or SEQ ID NO:22; (v) a set of LAMP primers including four primers including nucleic acid sequences having at least 90% sequence identity to the sequences of SEQ ID NO:25, SEQ ID NO:26, SEQ ID NO:27 or SEQ ID NO:28; or (vi) a set of LAMP primers including four primers including nucleic acid sequences having at least 90% sequence identity to the sequences of SEQ ID NO:31, SEQ ID NO:32, SEQ ID NO:37 or SEQ ID NO:34; thereby forming a SARS-CoV-2 amplification product; and (b) detecting the SARS-CoV-2 amplification product, thereby detecting the SARS-CoV-2 nucleic acid in the sample.

In another aspect is provided a method of detecting a SARS-CoV-2 nucleic acid in a sample, the method including: (a) contacting a sample with a set of loop-mediated isothermal amplification (LAMP) primers under conditions sufficient to amplify a SARS-CoV-2 nucleic acid, wherein the set of LAMP primers includes: (i) a set of LAMP primers including four primers including nucleic acid sequences having 90% sequence identity to the sequences of SEQ ID NO:1, SEQ ID NO:2, SEQ ID NO:3 or SEQ ID NO:4; (ii) a set of LAMP primers including four primers including nucleic acid sequences having 90% sequence identity to the sequences of SEQ ID NO:7, SEQ ID NO:8, SEQ ID NO:9 or SEQ ID NO:10; (iii) a set of LAMP primers including four primers including nucleic acid sequences having 90% sequence identity to the sequences of SEQ ID NO:13, SEQ ID NO:14, SEQ ID NO:15 or SEQ ID NO:16; (iv) a set of LAMP primers including four primers including nucleic acid sequences having 90% sequence identity to the sequences of SEQ ID NO:19, SEQ ID NO:20, SEQ ID NO:21 or SEQ ID NO:22; (v) a set of LAMP primers including four primers including nucleic acid sequences having 90% sequence identity to the sequences of SEQ ID NO:25, SEQ ID NO:26, SEQ ID NO:27 or SEQ ID NO:28; or (vi) a set of LAMP primers including four primers including nucleic acid sequences having 90% sequence identity to the sequences of SEQ ID NO:31, SEQ ID NO:32, SEQ ID NO:33 or SEQ ID NO:34 thereby forming a SARS-CoV-2 amplification product; and (b) detecting the SARS-CoV-2 amplification product, thereby detecting the SARS-CoV-2 nucleic acid in the sample.

In another aspect is provided a method of detecting a SARS-CoV-2 nucleic acid in a sample, the method including: (a) contacting a sample with a set of loop-mediated isothermal amplification (LAMP) primers under conditions sufficient to amplify a SARS-CoV-2 nucleic acid, wherein the set of LAMP primers includes: (i) a set of LAMP primers including four primers including nucleic acid sequences having 90% sequence identity to the sequences of SEQ ID NO:1, SEQ ID NO:2, SEQ ID NO:3 or SEQ ID NO:4; (ii) a set of LAMP primers including four primers including nucleic acid sequences having 90% sequence identity to the sequences of SEQ ID NO:7, SEQ ID NO:8, SEQ ID NO:9 or SEQ ID NO:10; (iii) a set of LAMP primers including four primers including nucleic acid sequences having 90% sequence identity to the sequences of SEQ ID NO:13, SEQ ID NO:14, SEQ ID NO:15 or SEQ ID NO:16; (iv) a set of LAMP primers including four primers including nucleic acid sequences having 90% sequence identity to the sequences of SEQ ID NO:19, SEQ ID NO:20, SEQ ID NO:21 or SEQ ID NO:22; (v) a set of LAMP primers including four primers including nucleic acid sequences having 90% sequence identity to the sequences of SEQ ID NO:25, SEQ ID NO:26, SEQ ID NO:27 or SEQ ID NO:28; or (vi) a set of LAMP primers including four primers including nucleic acid sequences having 90% sequence identity to the sequences of SEQ ID NO:31, SEQ ID NO:32, SEQ ID NO:37 or SEQ ID NO:34 thereby forming a SARS-CoV-2 amplification product; and (b) detecting the SARS-CoV-2 amplification product, thereby detecting the SARS-CoV-2 nucleic acid in the sample.

In embodiments, the set of loop-mediated isothermal amplification (LAMP) primers includes: (i) a set of LAMP primers including four primers including nucleic acid sequences having at least 95% sequence identity to the sequences of SEQ ID NO:1, SEQ ID NO:2, SEQ ID NO:3 or SEQ ID NO:4; (ii) a set of LAMP primers including four primers including nucleic acid sequences having at least 95% sequence identity to the sequences of SEQ ID NO:7, SEQ ID NO:8, SEQ ID NO:9 or SEQ ID NO:10; (iii) a set of LAMP primers including four primers including nucleic acid sequences having at least 95% sequence identity to the sequences of SEQ ID NO:13, SEQ ID NO:14, SEQ ID NO:15 or SEQ ID NO:16; (iv) a set of LAMP primers including four primers including nucleic acid sequences having at least 95% sequence identity to the sequences of SEQ ID NO:19, SEQ ID NO:20, SEQ ID NO:21 or SEQ ID NO:22; (v) a set of LAMP primers including four primers including nucleic acid sequences having at least 95% sequence identity to the sequences of SEQ ID NO:25, SEQ ID NO:26, SEQ ID NO:27 or SEQ ID NO:28; or (vi) a set of LAMP primers including four primers including nucleic acid sequences having at least 95% sequence identity to the sequences of SEQ ID NO:31, SEQ ID NO:32, SEQ ID NO:33 or SEQ ID NO:34.

In embodiments, the set of loop-mediated isothermal amplification (LAMP) primers includes: (i) a set of LAMP primers including four primers including nucleic acid sequences having at least 95% sequence identity to the sequences of SEQ ID NO:1, SEQ ID NO:2, SEQ ID NO:3 or SEQ ID NO:4; (ii) a set of LAMP primers including four primers including nucleic acid sequences having at least 95% sequence identity to the sequences of SEQ ID NO:7, SEQ ID NO:8, SEQ ID NO:9 or SEQ ID NO:10; (iii) a set of LAMP primers including four primers including nucleic acid sequences having at least 95% sequence identity to the sequences of SEQ ID NO:13, SEQ ID NO:14, SEQ ID NO:15 or SEQ ID NO:16; (iv) a set of LAMP primers including four primers including nucleic acid sequences having at least 95% sequence identity to the sequences of SEQ ID NO:19, SEQ ID NO:20, SEQ ID NO:21 or SEQ ID NO:22; (v) a set of LAMP primers including four primers including nucleic acid sequences having at least 95% sequence identity to the sequences of SEQ ID NO:25, SEQ ID NO:26, SEQ ID NO:27 or SEQ ID NO:28; or (vi) a set of LAMP primers including four primers including nucleic acid sequences having at least 95% sequence identity to the sequences of SEQ ID NO:31, SEQ ID NO:32, SEQ ID NO:37 or SEQ ID NO:34.

In embodiments, the set of loop-mediated isothermal amplification (LAMP) primers includes: (i) a set of LAMP primers including four primers including nucleic acid sequences having 95% sequence identity to the sequences of SEQ ID NO:1, SEQ ID NO:2, SEQ ID NO:3 or SEQ ID NO:4; (ii) a set of LAMP primers including four primers including nucleic acid sequences having 95% sequence identity to the sequences of SEQ ID NO:7, SEQ ID NO:8, SEQ ID NO:9 or SEQ ID NO:10; (iii) a set of LAMP primers including four primers including nucleic acid sequences having 95% sequence identity to the sequences of SEQ ID NO:13, SEQ ID NO:14, SEQ ID NO:15 or SEQ ID NO:16; (iv) a set of LAMP primers including four primers including nucleic acid sequences having 95% sequence identity to the sequences of SEQ ID NO:19, SEQ ID NO:20, SEQ ID NO:21 or SEQ ID NO:22; (v) a set of LAMP primers including four primers including nucleic acid sequences having 95% sequence identity to the sequences of SEQ ID NO:25, SEQ ID NO:26, SEQ ID NO:27 or SEQ ID NO:28; or (vi) a set of LAMP primers including four primers including nucleic acid sequences having 95% sequence identity to the sequences of SEQ ID NO:31, SEQ ID NO:32, SEQ ID NO:33 or SEQ ID NO:34.

In embodiments, the set of loop-mediated isothermal amplification (LAMP) primers includes: (i) a set of LAMP primers including four primers including nucleic acid sequences having 95% sequence identity to the sequences of SEQ ID NO:1, SEQ ID NO:2, SEQ ID NO:3 or SEQ ID NO:4; (ii) a set of LAMP primers including four primers including nucleic acid sequences having 95% sequence identity to the sequences of SEQ ID NO:7, SEQ ID NO:8, SEQ ID NO:9 or SEQ ID NO:10; (iii) a set of LAMP primers including four primers including nucleic acid sequences having 95% sequence identity to the sequences of SEQ ID NO:13, SEQ ID NO:14, SEQ ID NO:15 or SEQ ID NO:16; (iv) a set of LAMP primers including four primers including nucleic acid sequences having 95% sequence identity to the sequences of SEQ ID NO:19, SEQ ID NO:20, SEQ ID NO:21 or SEQ ID NO:22; (v) a set of LAMP primers including four primers including nucleic acid sequences having 95% sequence identity to the sequences of SEQ ID NO:25, SEQ ID NO:26, SEQ ID NO:27 or SEQ ID NO:28; or (vi) a set of LAMP primers including four primers including nucleic acid sequences having 95% sequence identity to the sequences of SEQ ID NO:31, SEQ ID NO:32, SEQ ID NO:37 or SEQ ID NO:34.

In embodiments, the set of loop-mediated isothermal amplification (LAMP) primers includes: (i) a set of LAMP primers including four primers including nucleic acid sequences having at least 98% sequence identity to the sequences of SEQ ID NO:1, SEQ ID NO:2, SEQ ID NO:3 or SEQ ID NO:4; (ii) a set of LAMP primers including four primers including nucleic acid sequences having at least 98% sequence identity to the sequences of SEQ ID NO:7, SEQ ID NO:8, SEQ ID NO:9 or SEQ ID NO:10; (iii) a set of LAMP primers including four primers including nucleic acid sequences having at least 98% sequence identity to the sequences of SEQ ID NO:13, SEQ ID NO:14, SEQ ID NO:15 or SEQ ID NO:16; (iv) a set of LAMP primers including four primers including nucleic acid sequences having at least 98% sequence identity to the sequences of SEQ ID NO:19, SEQ ID NO:20, SEQ ID NO:21 or SEQ ID NO:22; (v) a set of LAMP primers including four primers including nucleic acid sequences having at least 98% sequence identity to the sequences of SEQ ID NO:25, SEQ ID NO:26, SEQ ID NO:27 or SEQ ID NO:28; or (vi) a set of LAMP primers including four primers including nucleic acid sequences having at least 98% sequence identity to the sequences of SEQ ID NO:31, SEQ ID NO:32, SEQ ID NO:33 or SEQ ID NO:34.

In embodiments, the set of loop-mediated isothermal amplification (LAMP) primers includes: (i) a set of LAMP primers including four primers including nucleic acid sequences having at least 98% sequence identity to the sequences of SEQ ID NO:1, SEQ ID NO:2, SEQ ID NO:3 or SEQ ID NO:4; (ii) a set of LAMP primers including four primers including nucleic acid sequences having at least 98% sequence identity to the sequences of SEQ ID NO:7, SEQ ID NO:8, SEQ ID NO:9 or SEQ ID NO:10; (iii) a set of LAMP primers including four primers including nucleic acid sequences having at least 98% sequence identity to the sequences of SEQ ID NO:13, SEQ ID NO:14, SEQ ID NO:15 or SEQ ID NO:16; (iv) a set of LAMP primers including four primers including nucleic acid sequences having at least 98% sequence identity to the sequences of SEQ ID NO:19, SEQ ID NO:20, SEQ ID NO:21 or SEQ ID NO:22; (v) a set of LAMP primers including four primers including nucleic acid sequences having at least 98% sequence identity to the sequences of SEQ ID NO:25, SEQ ID NO:26, SEQ ID NO:27 or SEQ ID NO:28; or (vi) a set of LAMP primers including four primers including nucleic acid sequences having at least 98% sequence identity to the sequences of SEQ ID NO:31, SEQ ID NO:32, SEQ ID NO:37 or SEQ ID NO:34.

In embodiments, the set of loop-mediated isothermal amplification (LAMP) primers includes: (i) a set of LAMP primers including four primers including nucleic acid sequences having 98% sequence identity to the sequences of SEQ ID NO:1, SEQ ID NO:2, SEQ ID NO:3 or SEQ ID NO:4; (ii) a set of LAMP primers including four primers including nucleic acid sequences having 98% sequence identity to the sequences of SEQ ID NO:7, SEQ ID NO:8, SEQ ID NO:9 or SEQ ID NO:10; (iii) a set of LAMP primers including four primers including nucleic acid sequences having 98% sequence identity to the sequences of SEQ ID NO:13, SEQ ID NO:14, SEQ ID NO:15 or SEQ ID NO:16; (iv) a set of LAMP primers including four primers including nucleic acid sequences having 98% sequence identity to the sequences of SEQ ID NO:19, SEQ ID NO:20, SEQ ID NO:21 or SEQ ID NO:22; (v) a set of LAMP primers including four primers including nucleic acid sequences having 98% sequence identity to the sequences of SEQ ID NO:25, SEQ ID NO:26, SEQ ID NO:27 or SEQ ID NO:28; or (vi) a set of LAMP primers including four primers including nucleic acid sequences having 98% sequence identity to the sequences of SEQ ID NO:31, SEQ ID NO:32, SEQ ID NO:33 or SEQ ID NO:34.

In embodiments, the set of loop-mediated isothermal amplification (LAMP) primers includes: (i) a set of LAMP primers including four primers including nucleic acid sequences having 98% sequence identity to the sequences of SEQ ID NO:1, SEQ ID NO:2, SEQ ID NO:3 or SEQ ID NO:4; (ii) a set of LAMP primers including four primers including nucleic acid sequences having 98% sequence identity to the sequences of SEQ ID NO:7, SEQ ID NO:8, SEQ ID NO:9 or SEQ ID NO:10; (iii) a set of LAMP primers including four primers including nucleic acid sequences having 98% sequence identity to the sequences of SEQ ID NO:13, SEQ ID NO:14, SEQ ID NO:15 or SEQ ID NO:16; (iv) a set of LAMP primers including four primers including nucleic acid sequences having 98% sequence identity to the sequences of SEQ ID NO:19, SEQ ID NO:20, SEQ ID NO:21 or SEQ ID NO:22; (v) a set of LAMP primers including four primers including nucleic acid sequences having 98% sequence identity to the sequences of SEQ ID NO:25, SEQ ID NO:26, SEQ ID NO:27 or SEQ ID NO:28; or (vi) a set of LAMP primers including four primers including nucleic acid sequences having 98% sequence identity to the sequences of SEQ ID NO:31, SEQ ID NO:32, SEQ ID NO:37 or SEQ ID NO:34.

In embodiments, the set of loop-mediated isothermal amplification (LAMP) primers includes: (i) a set of LAMP primers including four primers including nucleic acid sequences having at least 99% sequence identity to the sequences of SEQ ID NO:1, SEQ ID NO:2, SEQ ID NO:3 or SEQ ID NO:4; (ii) a set of LAMP primers including four primers including nucleic acid sequences having at least 99% sequence identity to the sequences of SEQ ID NO:7, SEQ ID NO:8, SEQ ID NO:9 or SEQ ID NO:10; (iii) a set of LAMP primers including four primers including nucleic acid sequences having at least 99% sequence identity to the sequences of SEQ ID NO:13, SEQ ID NO:14, SEQ ID NO:15 or SEQ ID NO:16; (iv) a set of LAMP primers including four primers including nucleic acid sequences having at least 99% sequence identity to the sequences of SEQ ID NO:19, SEQ ID NO:20, SEQ ID NO:21 or SEQ ID NO:22; (v) a set of LAMP primers including four primers including nucleic acid sequences having at least 99% sequence identity to the sequences of SEQ ID NO:25, SEQ ID NO:26, SEQ ID NO:27 or SEQ ID NO:28; or (vi) a set of LAMP primers including four primers including nucleic acid sequences having at least 99% sequence identity to the sequences of SEQ ID NO:31, SEQ ID NO:32, SEQ ID NO:33 or SEQ ID NO:34.

In embodiments, the set of loop-mediated isothermal amplification (LAMP) primers includes: (i) a set of LAMP primers including four primers including nucleic acid sequences having at least 99% sequence identity to the sequences of SEQ ID NO:1, SEQ ID NO:2, SEQ ID NO:3 or SEQ ID NO:4; (ii) a set of LAMP primers including four primers including nucleic acid sequences having at least 99% sequence identity to the sequences of SEQ ID NO:7, SEQ ID NO:8, SEQ ID NO:9 or SEQ ID NO:10; (iii) a set of LAMP primers including four primers including nucleic acid sequences having at least 99% sequence identity to the sequences of SEQ ID NO:13, SEQ ID NO:14, SEQ ID NO:15 or SEQ ID NO:16; (iv) a set of LAMP primers including four primers including nucleic acid sequences having at least 99% sequence identity to the sequences of SEQ ID NO:19, SEQ ID NO:20, SEQ ID NO:21 or SEQ ID NO:22; (v) a set of LAMP primers including four primers including nucleic acid sequences having at least 99% sequence identity to the sequences of SEQ ID NO:25, SEQ ID NO:26, SEQ ID NO:27 or SEQ ID NO:28; or (vi) a set of LAMP primers including four primers including nucleic acid sequences having at least 99% sequence identity to the sequences of SEQ ID NO:31, SEQ ID NO:32, SEQ ID NO:37 or SEQ ID NO:34.

In embodiments, the set of loop-mediated isothermal amplification (LAMP) primers includes: (i) a set of LAMP primers including four primers including nucleic acid sequences having 99% sequence identity to the sequences of SEQ ID NO:1, SEQ ID NO:2, SEQ ID NO:3 or SEQ ID NO:4; (ii) a set of LAMP primers including four primers including nucleic acid sequences having 99% sequence identity to the sequences of SEQ ID NO:7, SEQ ID NO:8, SEQ ID NO:9 or SEQ ID NO:10; (iii) a set of LAMP primers including four primers including nucleic acid sequences having 99% sequence identity to the sequences of SEQ ID NO:13, SEQ ID NO:14, SEQ ID NO:15 or SEQ ID NO:16; (iv) a set of LAMP primers including four primers including nucleic acid sequences having 99% sequence identity to the sequences of SEQ ID NO:19, SEQ ID NO:20, SEQ ID NO:21 or SEQ ID NO:22; (v) a set of LAMP primers including four primers including nucleic acid sequences having 99% sequence identity to the sequences of SEQ ID NO:25, SEQ ID NO:26, SEQ ID NO:27 or SEQ ID NO:28; or (vi) a set of LAMP primers including four primers including nucleic acid sequences having 99% sequence identity to the sequences of SEQ ID NO:31, SEQ ID NO:32, SEQ ID NO:33 or SEQ ID NO:34.

In embodiments, the set of loop-mediated isothermal amplification (LAMP) primers includes: (i) a set of LAMP primers including four primers including nucleic acid sequences having 99% sequence identity to the sequences of SEQ ID NO:1, SEQ ID NO:2, SEQ ID NO:3 or SEQ ID NO:4; (ii) a set of LAMP primers including four primers including nucleic acid sequences having 99% sequence identity to the sequences of SEQ ID NO:7, SEQ ID NO:8, SEQ ID NO:9 or SEQ ID NO:10; (iii) a set of LAMP primers including four primers including nucleic acid sequences having 99% sequence identity to the sequences of SEQ ID NO:13, SEQ ID NO:14, SEQ ID NO:15 or SEQ ID NO:16; (iv) a set of LAMP primers including four primers including nucleic acid sequences having 99% sequence identity to the sequences of SEQ ID NO:19, SEQ ID NO:20, SEQ ID NO:21 or SEQ ID NO:22; (v) a set of LAMP primers including four primers including nucleic acid sequences having 99% sequence identity to the sequences of SEQ ID NO:25, SEQ ID NO:26, SEQ ID NO:27 or SEQ ID NO:28; or (vi) a set of LAMP primers including four primers including nucleic acid sequences having 99% sequence identity to the sequences of SEQ ID NO:31, SEQ ID NO:32, SEQ ID NO:37 or SEQ ID NO:34.

In embodiments, the set of loop-mediated isothermal amplification (LAMP) primers includes: (i) a set of LAMP primers including four primers including nucleic acid sequences having 100% sequence identity to the sequences of SEQ ID NO:1, SEQ ID NO:2, SEQ ID NO:3 or SEQ ID NO:4; (ii) a set of LAMP primers including four primers including nucleic acid sequences having 100% sequence identity to the sequences of SEQ ID NO:7, SEQ ID NO:8, SEQ ID NO:9 or SEQ ID NO:10; (iii) a set of LAMP primers including four primers including nucleic acid sequences having 100% sequence identity to the sequences of SEQ ID NO:13, SEQ ID NO:14, SEQ ID NO:15 or SEQ ID NO:16; (iv) a set of LAMP primers including four primers including nucleic acid sequences having 100% sequence identity to the sequences of SEQ ID NO:19, SEQ ID NO:20, SEQ ID NO:21 or SEQ ID NO:22; (v) a set of LAMP primers including four primers including nucleic acid sequences having 100% sequence identity to the sequences of SEQ ID NO:25, SEQ ID NO:26, SEQ ID NO:27 or SEQ ID NO:28; or (vi) a set of LAMP primers including four primers including nucleic acid sequences having 100% sequence identity to the sequences of SEQ ID NO:31, SEQ ID NO:32, SEQ ID NO:33 or SEQ ID NO:34.

In embodiments, the set of loop-mediated isothermal amplification (LAMP) primers includes: (i) a set of LAMP primers including four primers including nucleic acid sequences having 100% sequence identity to the sequences of SEQ ID NO:1, SEQ ID NO:2, SEQ ID NO:3 or SEQ ID NO:4; (ii) a set of LAMP primers including four primers including nucleic acid sequences having 100% sequence identity to the sequences of SEQ ID NO:7, SEQ ID NO:8, SEQ ID NO:9 or SEQ ID NO:10; (iii) a set of LAMP primers including four primers including nucleic acid sequences having 100% sequence identity to the sequences of SEQ ID NO:13, SEQ ID NO:14, SEQ ID NO:15 or SEQ ID NO:16; (iv) a set of LAMP primers including four primers including nucleic acid sequences having 100% sequence identity to the sequences of SEQ ID NO:19, SEQ ID NO:20, SEQ ID NO:21 or SEQ ID NO:22; (v) a set of LAMP primers including four primers including nucleic acid sequences having 100% sequence identity to the sequences of SEQ ID NO:25, SEQ ID NO:26, SEQ ID NO:27 or SEQ ID NO:28; or (vi) a set of LAMP primers including four primers including nucleic acid sequences having 100% sequence identity to the sequences of SEQ ID NO:31, SEQ ID NO:32, SEQ ID NO:37 or SEQ ID NO:34.

In another aspect is provided a method of detecting a SARS-CoV-2 nucleic acid in a sample, the method including: (a) contacting a sample with a set of loop-mediated isothermal amplification (LAMP) primers under conditions sufficient to amplify a SARS-CoV-2 nucleic acid, wherein the set of LAMP primers includes: (i) a set of LAMP primers including four primers including nucleic acid sequences having at least 90% sequence identity to the sequences of SEQ ID NO:1, SEQ ID NO:2, SEQ ID NO:3 and SEQ ID NO:4; (ii) a set of LAMP primers including four primers including nucleic acid sequences having at least 90% sequence identity to the sequences of SEQ ID NO:7, SEQ ID NO:8, SEQ ID NO:9 and SEQ ID NO:10; (iii) a set of LAMP primers including four primers including nucleic acid sequences having at least 90% sequence identity to the sequences of SEQ ID NO:13, SEQ ID NO:14, SEQ ID NO:15 and SEQ ID NO:16; (iv) a set of LAMP primers including four primers including nucleic acid sequences having at least 90% sequence identity to the sequences of SEQ ID NO:19, SEQ ID NO:20, SEQ ID NO:21 and SEQ ID NO:22; (v) a set of LAMP primers including four primers including nucleic acid sequences having at least 90% sequence identity to the sequences of SEQ ID NO:25, SEQ ID NO:26, SEQ ID NO:27 and SEQ ID NO:28; or (vi) a set of LAMP primers including four primers including nucleic acid sequences having at least 90% sequence identity to the sequences of SEQ ID NO:31, SEQ ID NO:32, SEQ ID NO:33 and SEQ ID NO:34; thereby forming a SARS-CoV-2 amplification product; and (b) detecting the SARS-CoV-2 amplification product, thereby detecting the SARS-CoV-2 nucleic acid in the sample.

In another aspect is provided a method of detecting a SARS-CoV-2 nucleic acid in a sample, the method including: (a) contacting a sample with a set of loop-mediated isothermal amplification (LAMP) primers under conditions sufficient to amplify a SARS-CoV-2 nucleic acid, wherein the set of LAMP primers includes: (i) a set of LAMP primers including four primers including nucleic acid sequences having at least 90% sequence identity to the sequences of SEQ ID NO:1, SEQ ID NO:2, SEQ ID NO:3 and SEQ ID NO:4; (ii) a set of LAMP primers including four primers including nucleic acid sequences having at least 90% sequence identity to the sequences of SEQ ID NO:7, SEQ ID NO:8, SEQ ID NO:9 and SEQ ID NO:10; (iii) a set of LAMP primers including four primers including nucleic acid sequences having at least 90% sequence identity to the sequences of SEQ ID NO:13, SEQ ID NO:14, SEQ ID NO:15 and SEQ ID NO:16; (iv) a set of LAMP primers including four primers including nucleic acid sequences having at least 90% sequence identity to the sequences of SEQ ID NO:19, SEQ ID NO:20, SEQ ID NO:21 and SEQ ID NO:22; (v) a set of LAMP primers including four primers including nucleic acid sequences having at least 90% sequence identity to the sequences of SEQ ID NO:25, SEQ ID NO:26, SEQ ID NO:27 and SEQ ID NO:28; or (vi) a set of LAMP primers including four primers including nucleic acid sequences having at least 90% sequence identity to the sequences of SEQ ID NO:31, SEQ ID NO:32, SEQ ID NO:37 and SEQ ID NO:34; thereby forming a SARS-CoV-2 amplification product; and (b) detecting the SARS-CoV-2 amplification product, thereby detecting the SARS-CoV-2 nucleic acid in the sample.

In embodiments, the set of LAMP primers in (i) further includes a set of LAMP primers including two primers including nucleic acid sequences having at least 90% sequence identity to the sequences of SEQ ID NO:5 or SEQ ID NO:6. In embodiments, the set of LAMP primers in (i) further includes a set of LAMP primers including two primers including nucleic acid sequences having at least 95% sequence identity to the sequences of SEQ ID NO:5 or SEQ ID NO:6. In embodiments, the set of LAMP primers in (i) further includes a set of LAMP primers including two primers including nucleic acid sequences having at least 98% sequence identity to the sequences of SEQ ID NO:5 or SEQ ID NO:6. In embodiments, the set of LAMP primers in (i) further includes a set of LAMP primers including two primers including nucleic acid sequences having at least 99% sequence identity to the sequences of SEQ ID NO:5 or SEQ ID NO:6.

In embodiments, the set of LAMP primers in (i) further includes a set of LAMP primers including two primers including nucleic acid sequences having 90% sequence identity to the sequences of SEQ ID NO:5 or SEQ ID NO:6. In embodiments, the set of LAMP primers in (i) further includes a set of LAMP primers including two primers including nucleic acid sequences having 95% sequence identity to the sequences of SEQ ID NO:5 or SEQ ID NO:6. In embodiments, the set of LAMP primers in (i) further includes a set of LAMP primers including two primers including nucleic acid sequences having 98% sequence identity to the sequences of SEQ ID NO:5 or SEQ ID NO:6. In embodiments, the set of LAMP primers in (i) further includes a set of LAMP primers including two primers including nucleic acid sequences having 99% sequence identity to the sequences of SEQ ID NO:5 or SEQ ID NO:6. In embodiments, the set of LAMP primers in (i) further includes a set of LAMP primers including two primers including nucleic acid sequences having 100% sequence identity to the sequences of SEQ ID NO:5 or SEQ ID NO:6.

In embodiments, the set of LAMP primers in (ii) further includes a set of LAMP primers including two primers including nucleic acid sequences having at least 90% sequence identity to the sequences of SEQ ID NO:11 or SEQ ID NO:12. In embodiments, the set of LAMP primers in (ii) further includes a set of LAMP primers including two primers including nucleic acid sequences having at least 95% sequence identity to the sequences of SEQ ID NO:11 or SEQ ID NO:12. In embodiments, the set of LAMP primers in (ii) further includes a set of LAMP primers including two primers including nucleic acid sequences having at least 98% sequence identity to the sequences of SEQ ID NO:11 or SEQ ID NO:12. In embodiments, the set of LAMP primers in (ii) further includes a set of LAMP primers including two primers including nucleic acid sequences having at least 99% sequence identity to the sequences of SEQ ID NO:11 or SEQ ID NO:12.

In embodiments, the set of LAMP primers in (ii) further includes a set of LAMP

primers including two primers including nucleic acid sequences having 90% sequence identity to the sequences of SEQ ID NO:11 or SEQ ID NO:12. In embodiments, the set of LAMP primers in (ii) further includes a set of LAMP primers including two primers including nucleic acid sequences having 95% sequence identity to the sequences of SEQ ID NO:11 or SEQ ID NO:12. In embodiments, the set of LAMP primers in (ii) further includes a set of LAMP primers including two primers including nucleic acid sequences having 98% sequence identity to the sequences of SEQ ID NO:11 or SEQ ID NO:12. In embodiments, the set of LAMP primers in (ii) further includes a set of LAMP primers including two primers including nucleic acid sequences having 99% sequence identity to the sequences of SEQ ID NO:11 or SEQ ID NO:12. In embodiments, the set of LAMP primers in (ii) further includes a set of LAMP primers including two primers including nucleic acid sequences having 100% sequence identity to the sequences of SEQ ID NO:11 or SEQ ID NO:12.

In embodiments, the set of LAMP primers in (iii) further includes a set of LAMP primers including two primers including nucleic acid sequences having at least 90% sequence identity to the sequences of SEQ ID NO:17 or SEQ ID NO:18. In embodiments, the set of LAMP primers in (iii) further includes a set of LAMP primers including two primers including nucleic acid sequences having at least 95% sequence identity to the sequences of SEQ ID NO:17 or SEQ ID NO:18. In embodiments, the set of LAMP primers in (iii) further includes a set of LAMP primers including two primers including nucleic acid sequences having at least 98% sequence identity to the sequences of SEQ ID NO:17 or SEQ ID NO:18. In embodiments, the set of LAMP primers in (iii) further includes a set of LAMP primers including two primers including nucleic acid sequences having at least 99% sequence identity to the sequences of SEQ ID NO:17 or SEQ ID NO:18.

In embodiments, the set of LAMP primers in (iii) further includes a set of LAMP primers including two primers including nucleic acid sequences having 90% sequence identity to the sequences of SEQ ID NO:17 or SEQ ID NO:18. In embodiments, the set of LAMP primers in (iii) further includes a set of LAMP primers including two primers including nucleic acid sequences having 95% sequence identity to the sequences of SEQ ID NO:17 or SEQ ID NO:18. In embodiments, the set of LAMP primers in (iii) further includes a set of LAMP primers including two primers including nucleic acid sequences having 98% sequence identity to the sequences of SEQ ID NO:17 or SEQ ID NO:18. In embodiments, the set of LAMP primers in (iii) further includes a set of LAMP primers including two primers including nucleic acid sequences having 99% sequence identity to the sequences of SEQ ID NO:17 or SEQ ID NO:18. In embodiments, the set of LAMP primers in (iii) further includes a set of LAMP primers including two primers including nucleic acid sequences having 100% sequence identity to the sequences of SEQ ID NO:17 or SEQ ID NO:18.

In embodiments, the set of LAMP primers in (iv) further includes a set of LAMP

primers including two primers including nucleic acid sequences having at least 90% sequence identity to the sequences of SEQ ID NO:23 or SEQ ID NO:24. In embodiments, the set of LAMP primers in (iv) further includes a set of LAMP primers including two primers including nucleic acid sequences having at least 95% sequence identity to the sequences of SEQ ID NO:23 or SEQ ID NO:24. In embodiments, the set of LAMP primers in (iv) further includes a set of LAMP primers including two primers including nucleic acid sequences having at least 98% sequence identity to the sequences of SEQ ID NO:23 or SEQ ID NO:24.

In embodiments, the set of LAMP primers in (iv) further includes a set of LAMP primers including two primers including nucleic acid sequences having at least 99% sequence identity to the sequences of SEQ ID NO:23 or SEQ ID NO:24.

In embodiments, the set of LAMP primers in (iv) further includes a set of LAMP primers including two primers including nucleic acid sequences having 90% sequence identity to the sequences of SEQ ID NO:23 or SEQ ID NO:24. In embodiments, the set of LAMP primers in (iv) further includes a set of LAMP primers including two primers including nucleic acid sequences having 95% sequence identity to the sequences of SEQ ID NO:23 or SEQ ID NO:24. In embodiments, the set of LAMP primers in (iv) further includes a set of LAMP primers including two primers including nucleic acid sequences having 98% sequence identity to the sequences of SEQ ID NO:23 or SEQ ID NO:24. In embodiments, the set of LAMP primers in (iv) further includes a set of LAMP primers including two primers including nucleic acid sequences having 99% sequence identity to the sequences of SEQ ID NO:23 or SEQ ID NO:24. In embodiments, the set of LAMP primers in (iv) further includes a set of LAMP primers including two primers including nucleic acid sequences having 100% sequence identity to the sequences of SEQ ID NO:23 or SEQ ID NO:24.

In embodiments, the set of LAMP primers in (iv) further includes a set of LAMP primers including two primers including nucleic acid sequences having at least 90% sequence identity to the sequences of SEQ ID NO:29 or SEQ ID NO:30. In embodiments, the set of LAMP primers in (iv) further includes a set of LAMP primers including two primers including nucleic acid sequences having at least 95% sequence identity to the sequences of SEQ ID NO:29 or SEQ ID NO:30. In embodiments, the set of LAMP primers in (iv) further includes a set of LAMP primers including two primers including nucleic acid sequences having at least 98% sequence identity to the sequences of SEQ ID NO:29 or SEQ ID NO:30. In embodiments, the set of LAMP primers in (iv) further includes a set of LAMP primers including two primers including nucleic acid sequences having at least 99% sequence identity to the sequences of SEQ ID NO:29 or SEQ ID NO:30.

In embodiments, the set of LAMP primers in (iv) further includes a set of LAMP primers including two primers including nucleic acid sequences having 90% sequence identity to the sequences of SEQ ID NO:29 or SEQ ID NO:30. In embodiments, the set of LAMP primers in (iv) further includes a set of LAMP primers including two primers including nucleic acid sequences having 95% sequence identity to the sequences of SEQ ID NO:29 or SEQ ID NO:30. In embodiments, the set of LAMP primers in (iv) further includes a set of LAMP primers including two primers including nucleic acid sequences having 98% sequence identity to the sequences of SEQ ID NO:29 or SEQ ID NO:30. In embodiments, the set of LAMP primers in (iv) further includes a set of LAMP primers including two primers including nucleic acid sequences having 99% sequence identity to the sequences of SEQ ID NO:29 or SEQ ID NO:30. In embodiments, the set of LAMP primers in (iv) further includes a set of LAMP primers including two primers including nucleic acid sequences having 100% sequence identity to the sequences of SEQ ID NO:29 or SEQ ID NO:30.

In embodiments, the set of LAMP primers in (iv) further includes a set of LAMP primers including two primers including nucleic acid sequences having at least 90% sequence identity to the sequences of SEQ ID NO:35 or SEQ ID NO:36. In embodiments, the set of LAMP primers in (iv) further includes a set of LAMP primers including two primers including nucleic acid sequences having at least 95% sequence identity to the sequences of SEQ ID NO:35 or SEQ ID NO:36. In embodiments, the set of LAMP primers in (iv) further includes a set of LAMP primers including two primers including nucleic acid sequences having at least 98% sequence identity to the sequences of SEQ ID NO:35 or SEQ ID NO:36. In embodiments, the set of LAMP primers in (iv) further includes a set of LAMP primers including two primers including nucleic acid sequences having at least 99% sequence identity to the sequences of SEQ ID NO:35 or SEQ ID NO:36.

In embodiments, the set of LAMP primers in (iv) further includes a set of LAMP

primers including two primers including nucleic acid sequences having 90% sequence identity to the sequences of SEQ ID NO:35 or SEQ ID NO:36. In embodiments, the set of LAMP primers in (iv) further includes a set of LAMP primers including two primers including nucleic acid sequences having 95% sequence identity to the sequences of SEQ ID NO:35 or SEQ ID NO:36. In embodiments, the set of LAMP primers in (iv) further includes a set of LAMP primers including two primers including nucleic acid sequences having 98% sequence identity to the sequences of SEQ ID NO:35 or SEQ ID NO:36. In embodiments, the set of LAMP primers in (iv) further includes a set of LAMP primers including two primers including nucleic acid sequences having 99% sequence identity to the sequences of SEQ ID NO:35 or SEQ ID NO:36. In embodiments, the set of LAMP primers in (iv) further includes a set of LAMP primers including two primers including nucleic acid sequences having 100% sequence identity to the sequences of SEQ ID NO:35 or SEQ ID NO:36.

In another aspect is provided a method of detecting a SARS-CoV-2 nucleic acid in a sample, the method including: (a) contacting a sample with a set of loop-mediated isothermal amplification (LAMP) primers under conditions sufficient to amplify a SARS-CoV-2 nucleic acid, wherein the set of LAMP primers includes: four primers including nucleic acid sequences having at least 90% sequence identity to the sequences of SEQ ID NO:1, SEQ ID NO:2, SEQ ID NO:3 or SEQ ID NO:4, thereby forming a SARS-CoV-2 amplification product; and (b) detecting the SARS-CoV-2 amplification product, thereby detecting the SARS-CoV-2 nucleic acid in the sample. In embodiments, the set of LAMP primers further includes a set of LAMP primers including two primers including nucleic acid sequences having at least 90% sequence identity to the sequences of SEQ ID NO:5 or SEQ ID NO:6. In one embodiment, the set of LAMP primers includes a set of LAMP primers including six primers including nucleic acid sequences having at least 90% sequence identity to the sequences of SEQ ID NO:1, SEQ ID NO:2, SEQ ID NO:3, SEQ ID NO:4, SEQ ID NO:5 or SEQ ID NO:6. In embodiments, the at least 90% sequence identity is 91%, 92%, 93%, 94%, 95%, 96%, 97%, 98%, 99% or 100%.

In another aspect is provided a method of detecting a SARS-CoV-2 nucleic acid in a sample, the method including: (a) contacting a sample with a set of loop-mediated isothermal amplification (LAMP) primers under conditions sufficient to amplify a SARS-CoV-2 nucleic acid, wherein the set of LAMP primers includes: four primers including nucleic acid sequences having at least 90% sequence identity to the sequences of SEQ ID NO:1, SEQ ID NO:2, SEQ ID NO:3 and SEQ ID NO:4, thereby forming a SARS-CoV-2 amplification product; and (b) detecting the SARS-CoV-2 amplification product, thereby detecting the SARS-CoV-2 nucleic acid in the sample. In embodiments, the set of LAMP primers further includes a set of LAMP primers including two primers including nucleic acid sequences having at least 90% sequence identity to the sequences of SEQ ID NO:5 and SEQ ID NO:6.

In one embodiment, the set of LAMP primers includes a set of LAMP primers including six primers including nucleic acid sequences having at least 90% sequence identity to the sequences of SEQ ID NO:1, SEQ ID NO:2, SEQ ID NO:3, SEQ ID NO:4, SEQ ID NO:5 and SEQ ID NO:6. In embodiments, the at least 90% sequence identity is 91%, 92%, 93%, 94%, 95%, 96%, 97%, 98%, 99% or 100%. In one embodiment, the set of LAMP primers includes a set of LAMP primers including six primers with the sequences of SEQ ID NO:1, SEQ ID NO:2, SEQ ID NO:3, SEQ ID NO:4, SEQ ID NO:5 and SEQ ID NO:6.

In another aspect is provided a method of detecting a SARS-CoV-2 nucleic acid in a sample, the method including: (a) contacting a sample with a set of loop-mediated isothermal amplification (LAMP) primers under conditions sufficient to amplify a SARS-CoV-2 nucleic acid, wherein the set of LAMP primers includes: four primers including nucleic acid sequences having at least 90% sequence identity to the sequences of SEQ ID NO:7, SEQ ID NO:8, SEQ ID NO:9 or SEQ ID NO:10, thereby forming a SARS-CoV-2 amplification product; and (b) detecting the SARS-CoV-2 amplification product, thereby detecting the SARS-CoV-2 nucleic acid in the sample. In embodiments, the set of LAMP primers further includes a set of LAMP primers including two primers including nucleic acid sequences having at least 90% sequence identity to the sequences of SEQ ID NO:11 or SEQ ID NO:12.

In one embodiment, the set of LAMP primers includes a set of LAMP primers including six primers including nucleic acid sequences having at least 90% sequence identity to the sequences of SEQ ID NO:7, SEQ ID NO:8, SEQ ID NO:9, SEQ ID NO:10, SEQ ID NO:11 or SEQ ID NO:12. In embodiments, the at least 90% sequence identity is 91%, 92%, 93%, 94%, 95%, 96%, 97%, 98%, 99% or 100%.

In another aspect is provided a method of detecting a SARS-CoV-2 nucleic acid in a sample, the method including: (a) contacting a sample with a set of loop-mediated isothermal amplification (LAMP) primers under conditions sufficient to amplify a SARS-CoV-2 nucleic acid, wherein the set of LAMP primers includes: four primers including nucleic acid sequences having at least 90% sequence identity to the sequences of SEQ ID NO:7, SEQ ID NO:8, SEQ ID NO:9 and SEQ ID NO:10, thereby forming a SARS-CoV-2 amplification product; and (b) detecting the SARS-CoV-2 amplification product, thereby detecting the SARS-CoV-2 nucleic acid in the sample. In embodiments, the set of LAMP primers further includes a set of LAMP primers including two primers including nucleic acid sequences having at least 90% sequence identity to the sequences of SEQ ID NO:11 and SEQ ID NO:12. In one embodiment, the set of LAMP primers includes a set of LAMP primers including six primers including nucleic acid sequences having at least 90% sequence identity to the sequences of SEQ ID NO:7, SEQ ID NO:8, SEQ ID NO:9, SEQ ID NO:10, SEQ ID NO:11 and SEQ ID NO:12. In embodiments, the at least 90% sequence identity is 91%, 92%, 93%, 94%, 95%, 96%, 97%, 98%, 99% or 100%. In one embodiment, the set of LAMP primers includes a set of LAMP primers including six primers with the sequences of SEQ ID NO:7, SEQ ID NO:8, SEQ ID NO:9, SEQ ID NO:10, SEQ ID NO:11 and SEQ ID NO:12.

In another aspect is provided a method of detecting a SARS-CoV-2 nucleic acid in a sample, the method including: (a) contacting a sample with a set of loop-mediated isothermal amplification (LAMP) primers under conditions sufficient to amplify a SARS-CoV-2 nucleic acid, wherein the set of LAMP primers includes: four primers including nucleic acid sequences having at least 90% sequence identity to the sequences of SEQ ID NO:13, SEQ ID NO:14, SEQ ID NO:15 or SEQ ID NO:16, thereby forming a SARS-CoV-2 amplification product; and (b) detecting the SARS-CoV-2 amplification product, thereby detecting the SARS-CoV-2 nucleic acid in the sample. In embodiments, the set of LAMP primers further includes a set of LAMP primers including two primers including nucleic acid sequences having at least 90% sequence identity to the sequences of SEQ ID NO:17 or SEQ ID NO:18.

In one embodiment, the set of LAMP primers includes a set of LAMP primers including six primers including nucleic acid sequences having at least 90% sequence identity to the sequences of SEQ ID NO:13, SEQ ID NO:14, SEQ ID NO:15, SEQ ID NO:16, SEQ ID NO:17 or SEQ ID NO:18. In embodiments, the at least 90% sequence identity is 91%, 92%, 93%, 94%, 95%, 96%, 97%, 98%, 99% or 100%.

In another aspect is provided a method of detecting a SARS-CoV-2 nucleic acid in a sample, the method including: (a) contacting a sample with a set of loop-mediated isothermal amplification (LAMP) primers under conditions sufficient to amplify a SARS-CoV-2 nucleic acid, wherein the set of LAMP primers includes: four primers including nucleic acid sequences having at least 90% sequence identity to the sequences of SEQ ID NO:13, SEQ ID NO:14, SEQ ID NO:15 and SEQ ID NO:16, thereby forming a SARS-CoV-2 amplification product; and (b) detecting the SARS-CoV-2 amplification product, thereby detecting the SARS-CoV-2 nucleic acid in the sample. In embodiments, the set of LAMP primers further includes a set of LAMP primers including two primers including nucleic acid sequences having at least 90% sequence identity to the sequences of SEQ ID NO:17 and SEQ ID NO:18. In one embodiment, the set of LAMP primers includes a set of LAMP primers including six primers including nucleic acid sequences having at least 90% sequence identity to the sequences of SEQ ID NO:13, SEQ ID NO:14, SEQ ID NO:15, SEQ ID NO:16, SEQ ID NO:17 and SEQ ID NO:18. In embodiments, the at least 90% sequence identity is 91%, 92%, 93%, 94%, 95%, 96%, 97%, 98%, 99% or 100%. In one embodiment, the set of LAMP primers includes a set of LAMP primers including six primers with the sequences of SEQ ID NO:13, SEQ ID NO:14, SEQ ID NO:15, SEQ ID NO:16, SEQ ID NO:17 and SEQ ID NO:18.

In another aspect is provided a method of detecting a SARS-CoV-2 nucleic acid in a sample, the method including: (a) contacting a sample with a set of loop-mediated isothermal amplification (LAMP) primers under conditions sufficient to amplify a SARS-CoV-2 nucleic acid, wherein the set of LAMP primers includes: four primers including nucleic acid sequences having at least 90% sequence identity to the sequences of SEQ ID NO:19, SEQ ID NO:20, SEQ ID NO:21 or SEQ ID NO:22, thereby forming a SARS-CoV-2 amplification product; and (b) detecting the SARS-CoV-2 amplification product, thereby detecting the SARS-CoV-2 nucleic acid in the sample. In embodiments, the set of LAMP primers further includes a set of LAMP primers including two primers including nucleic acid sequences having at least 90% sequence identity to the sequences of SEQ ID NO:23 or SEQ ID NO:24. In one embodiment, the set of LAMP primers includes a set of LAMP primers including six primers including nucleic acid sequences having at least 90% sequence identity to the sequences of SEQ ID NO:19, SEQ ID NO:20, SEQ ID NO:21, SEQ ID NO:22, SEQ ID NO:23 or SEQ ID NO:24. In embodiments, the at least 90% sequence identity is 91%, 92%, 93%, 94%, 95%, 96%, 97%, 98%, 99% or 100%.

In another aspect is provided a method of detecting a SARS-CoV-2 nucleic acid in a sample, the method including: (a) contacting a sample with a set of loop-mediated isothermal amplification (LAMP) primers under conditions sufficient to amplify a SARS-CoV-2 nucleic acid, wherein the set of LAMP primers includes: four primers including nucleic acid sequences having at least 90% sequence identity to the sequences of SEQ ID NO:19, SEQ ID NO:20, SEQ ID NO:21 and SEQ ID NO:22, thereby forming a SARS-CoV-2 amplification product; and (b) detecting the SARS-CoV-2 amplification product, thereby detecting the SARS-CoV-2 nucleic acid in the sample. In embodiments, the set of LAMP primers further includes a set of LAMP primers including two primers including nucleic acid sequences having at least 90% sequence identity to the sequences of SEQ ID NO:23 and SEQ ID NO:24. In one embodiment, the set of LAMP primers includes a set of LAMP primers including six primers including nucleic acid sequences having at least 90% sequence identity to the sequences of SEQ ID NO:19, SEQ ID NO:20, SEQ ID NO:21, SEQ ID NO:22, SEQ ID NO:23 and SEQ ID NO:24. In embodiments, the at least 90% sequence identity is 91%, 92%, 93%, 94%, 95%, 96%, 97%, 98%, 99% or 100%. In one embodiment, the set of LAMP primers includes a set of LAMP primers including six primers with the sequences of SEQ ID NO:19, SEQ ID NO:20, SEQ ID NO:21, SEQ ID NO:22, SEQ ID NO:23 and SEQ ID NO:24.

In another aspect is provided a method of detecting a SARS-CoV-2 nucleic acid in a sample, the method including: (a) contacting a sample with a set of loop-mediated isothermal amplification (LAMP) primers under conditions sufficient to amplify a SARS-CoV-2 nucleic acid, wherein the set of LAMP primers includes: four primers including nucleic acid sequences having at least 90% sequence identity to the sequences of SEQ ID NO:25, SEQ ID NO:26, SEQ ID NO:27 or SEQ ID NO:28, thereby forming a SARS-CoV-2 amplification product; and (b) detecting the SARS-CoV-2 amplification product, thereby detecting the SARS-CoV-2 nucleic acid in the sample. In embodiments, the set of LAMP primers further includes a set of LAMP primers including two primers including nucleic acid sequences having at least 90% sequence identity to the sequences of SEQ ID NO:29 or SEQ ID NO:30. In one embodiment, the set of LAMP primers includes a set of LAMP primers including six primers including nucleic acid sequences having at least 90% sequence identity to the sequences of SEQ ID NO:25, SEQ ID NO:26, SEQ ID NO:27, SEQ ID NO:28, SEQ ID NO:29 or SEQ ID NO:30. In embodiments, the at least 90% sequence identity is 91%, 92%, 93%, 94%, 95%, 96%, 97%, 98%, 99% or 100%.

In another aspect is provided a method of detecting a SARS-CoV-2 nucleic acid in a sample, the method including: (a) contacting a sample with a set of loop-mediated isothermal amplification (LAMP) primers under conditions sufficient to amplify a SARS-CoV-2 nucleic acid, wherein the set of LAMP primers includes: four primers including nucleic acid sequences having at least 90% sequence identity to the sequences of SEQ ID NO:25, SEQ ID NO:26, SEQ ID NO:27 and SEQ ID NO:28, thereby forming a SARS-CoV-2 amplification product; and (b) detecting the SARS-CoV-2 amplification product, thereby detecting the SARS-CoV-2 nucleic acid in the sample. In embodiments, the set of LAMP primers further includes a set of LAMP primers including two primers including nucleic acid sequences having at least 90% sequence identity to the sequences of SEQ ID NO:29 and SEQ ID NO:30. In one embodiment, the set of LAMP primers includes a set of LAMP primers including six primers including nucleic acid sequences having at least 90% sequence identity to the sequences of SEQ ID NO:25, SEQ ID NO:26, SEQ ID NO:27, SEQ ID NO:28, SEQ ID NO:29 and SEQ ID NO:30. In embodiments, the at least 90% sequence identity is 91%, 92%, 93%, 94%, 95%, 96%, 97%, 98%, 99% or 100%. In one embodiment, the set of LAMP primers includes a set of LAMP primers including six primers with the sequences of SEQ ID NO:25, SEQ ID NO:26, SEQ ID NO:27, SEQ ID NO:28, SEQ ID NO:29 and SEQ ID NO:30.

In another aspect is provided a method of detecting a SARS-CoV-2 nucleic acid in a sample, the method including: (a) contacting a sample with a set of loop-mediated isothermal amplification (LAMP) primers under conditions sufficient to amplify a SARS-CoV-2 nucleic acid, wherein the set of LAMP primers includes: four primers including nucleic acid sequences having at least 90% sequence identity to the sequences of SEQ ID NO:31, SEQ ID NO:32, SEQ ID NO:33 or SEQ ID NO:34, thereby forming a SARS-CoV-2 amplification product; and (b) detecting the SARS-CoV-2 amplification product, thereby detecting the SARS-CoV-2 nucleic acid in the sample. In embodiments, the set of LAMP primers further includes a set of LAMP primers including two primers including nucleic acid sequences having at least 90% sequence identity to the sequences of SEQ ID NO:35 or SEQ ID NO:36. In one embodiment, the set of LAMP primers includes a set of LAMP primers including six primers including nucleic acid sequences having at least 90% sequence identity to the sequences of SEQ ID NO:31, SEQ ID NO:32, SEQ ID NO:33, SEQ ID NO:34, SEQ ID NO:35 or SEQ ID NO:36. In embodiments, the at least 90% sequence identity is 91%, 92%, 93%, 94%, 95%, 96%, 97%, 98%, 99% or 100%.

In another aspect is provided a method of detecting a SARS-CoV-2 nucleic acid in a sample, the method including: (a) contacting a sample with a set of loop-mediated isothermal amplification (LAMP) primers under conditions sufficient to amplify a SARS-CoV-2 nucleic acid, wherein the set of LAMP primers includes: four primers including nucleic acid sequences having at least 90% sequence identity to the sequences of SEQ ID NO:31, SEQ ID NO:32, SEQ ID NO:33 and SEQ ID NO:34, thereby forming a SARS-CoV-2 amplification product; and (b) detecting the SARS-CoV-2 amplification product, thereby detecting the SARS-CoV-2 nucleic acid in the sample. In embodiments, the set of LAMP primers further includes a set of LAMP primers including two primers including nucleic acid sequences having at least 90% sequence identity to the sequences of SEQ ID NO:35 and SEQ ID NO:36. In one embodiment, the set of LAMP primers includes a set of LAMP primers including six primers including nucleic acid sequences having at least 90% sequence identity to the sequences of SEQ ID NO:31, SEQ ID NO:32, SEQ ID NO:33, SEQ ID NO:34, SEQ ID NO:35 and SEQ ID NO:36. In embodiments, the at least 90% sequence identity is 91%, 92%, 93%, 94%, 95%, 96%, 97%, 98%, 99% or 100%. In one embodiment, the set of LAMP primers includes a set of LAMP primers including six primers with the sequences of SEQ ID NO:31, SEQ ID NO:32, SEQ ID NO:33, SEQ ID NO:34, SEQ ID NO:35 and SEQ ID NO:36.

In another aspect is provided a method of detecting a SARS-CoV-2 nucleic acid in a sample, the method including: (a) contacting a sample with a set of loop-mediated isothermal amplification (LAMP) primers under conditions sufficient to amplify a SARS-CoV-2 nucleic acid, wherein the set of LAMP primers includes: four primers including nucleic acid sequences having at least 90% sequence identity to the sequences of SEQ ID NO:31, SEQ ID NO:32, SEQ ID NO:37 or SEQ ID NO:34, thereby forming a SARS-CoV-2 amplification product; and (b) detecting the SARS-CoV-2 amplification product, thereby detecting the SARS-CoV-2 nucleic acid in the sample. In embodiments, the set of LAMP primers further includes a set of LAMP primers including two primers including nucleic acid sequences having at least 90% sequence identity to the sequences of SEQ ID NO:35 or SEQ ID NO:36. In one embodiment, the set of LAMP primers includes a set of LAMP primers including six primers including nucleic acid sequences having at least 90% sequence identity to the sequences of SEQ ID NO:31, SEQ ID NO:32, SEQ ID NO:37, SEQ ID NO:34, SEQ ID NO:35 or SEQ ID NO:36. In embodiments, the at least 90% sequence identity is 91%, 92%, 93%, 94%, 95%, 96%, 97%, 98%, 99% or 100%.

In another aspect is provided a method of detecting a SARS-CoV-2 nucleic acid in a sample, the method including: (a) contacting a sample with a set of loop-mediated isothermal amplification (LAMP) primers under conditions sufficient to amplify a SARS-CoV-2 nucleic acid, wherein the set of LAMP primers includes: four primers including nucleic acid sequences having at least 90% sequence identity to the sequences of SEQ ID NO:31, SEQ ID NO:32, SEQ ID NO:37 and SEQ ID NO:34, thereby forming a SARS-CoV-2 amplification product; and (b) detecting the SARS-CoV-2 amplification product, thereby detecting the SARS-CoV-2 nucleic acid in the sample. In embodiments, the set of LAMP primers further includes a set of LAMP primers including two primers including nucleic acid sequences having at least 90% sequence identity to the sequences of SEQ ID NO:35 and SEQ ID NO:36. In one embodiment, the set of LAMP primers includes a set of LAMP primers including six primers including nucleic acid sequences having at least 90% sequence identity to the sequences of SEQ ID NO:31, SEQ ID NO:32, SEQ ID NO:37, SEQ ID NO:34, SEQ ID NO:35 and SEQ ID NO:36. In embodiments, the at least 90% sequence identity is 91%, 92%, 93%, 94%, 95%, 96%, 97%, 98%, 99% or 100%. In one embodiment, the set of LAMP primers includes a set of LAMP primers including six primers with the sequences of SEQ ID NO:31, SEQ ID NO:32, SEQ ID NO:37, SEQ ID NO:34, SEQ ID NO:35 and SEQ ID NO:36.

In embodiments, the set of LAMP primers in (i) further includes a set of LAMP primers including two primers including nucleic acid sequences having at least 90% sequence identity to the sequences of SEQ ID NO:5 or SEQ ID NO:6.

In embodiments, the set of LAMP primers in (ii) further includes a set of LAMP primers including two primers including nucleic acid sequences having at least 90% sequence identity to the sequences of SEQ ID NO:11 or SEQ ID NO:12.

In embodiments, the set of LAMP primers in (iii) further includes a set of LAMP primers including two primers including nucleic acid sequences having at least 90% sequence identity to the sequences of SEQ ID NO:17 or SEQ ID NO:18.

In embodiments, the set of LAMP primers in (iv) further includes a set of LAMP

primers including two primers including nucleic acid sequences having at least 90% sequence identity to the sequences of SEQ ID NO:23 or SEQ ID NO:24.

In embodiments, the set of LAMP primers in (iv) further includes a set of LAMP primers including two primers including nucleic acid sequences having at least 90% sequence identity to the sequences of SEQ ID NO:29 or SEQ ID NO:30.

In embodiments, the set of LAMP primers in (iv) further includes a set of LAMP primers including two primers including nucleic acid sequences having at least 90% sequence identity to the sequences of SEQ ID NO:35 or SEQ ID NO:36.

In embodiments, the SARS-CoV-2 nucleic acid encodes a 5′-UTR/ORF1ab, a spike protein, nucleocapsid gene or a functional fragment thereof. In embodiments, the SARS-CoV-2 nucleic acid encodes a 5′-UTR/ORF1ab or a functional fragment thereof. In embodiments, the SARS-CoV-2 nucleic acid encodes a spike protein or a functional fragment thereof. In embodiments, the SARS-CoV-2 nucleic acid encodes a nucleocapsid gene or a functional fragment thereof.

In embodiments, the at least 90% sequence identity is 95%. In embodiments, the at least 90% sequence identity is 98%. In embodiments, the at least 90% sequence identity is 100%. In embodiments, the sample is saliva. In embodiments, the sample have been isolated from a subject having or being at risk of having COVID-19.

For the kits or methods of detection provided herein each occurrence were the primer includes or has the sequence of SEQ ID NO:33, the primer may include or have the sequence of SEQ ID NO:37 or SEQ ID NO:38. Therefore, the sequences of SEQ ID NO:33, SEQ ID NO:37 and SEQ ID NO:38 are used herein interchangeably and may be interchanged for each other for the kits and methods provided herein including embodiments thereof. For example, in an aspect where a kit includes a primer including the sequence of SEQ ID NO:33, a person having ordinary skill in the art would recognize that the same kit may be used with a primer including the sequence of SEQ ID NO:37 or primer including the sequence of SEQ ID NO:38.

The primers used for the kits and methods provided herein (e.g., primers including the sequences of SEQ ID NO:1-SEQ ID NO:38) may include any sequence that has a sequence identity of at least 90% to any of the sequences of SEQ ID NO:1-SEQ ID NO:38. In embodiments, the LAMP primer includes a sequence wherein the sequence has 1, 2, 3, 4, 5 6, 7, 8 or 9 nucleotides removed from the 5′ end or the 3′ end or 5′ end and the 3′ end of the sequence of SEQ ID NO:1-SEQ ID NO:38.

In embodiments, the at least 90% sequence identity is 91%. In embodiments, the at least 90% sequence identity is 92%. In embodiments, the at least 90% sequence identity is 93%. In embodiments, the at least 90% sequence identity is 94%. In embodiments, the at least 90% sequence identity is 95%. In embodiments, the at least 90% sequence identity is 96%. In embodiments, the at least 90% sequence identity is 97%. In embodiments, the at least 90% sequence identity is 98%. In embodiments, the at least 90% sequence identity is 99%. In embodiments, the at least 90% sequence identity is 100%.

In embodiments, the at least 90% sequence identity is 91% sequence identity to 10-consecutive nucleotides. In embodiments, the at least 90% sequence identity is 92% sequence identity to 10-40 consecutive nucleotides. In embodiments, the at least 90% sequence identity is 93% sequence identity to 10-40 consecutive nucleotides. In embodiments, the at least 90% sequence identity is 94% sequence identity to 10-40 consecutive nucleotides. In embodiments, the at least 90% sequence identity is 95% sequence identity to 10-40 consecutive nucleotides. In embodiments, the at least 90% sequence identity is 96% sequence identity to 10-40 consecutive nucleotides. In embodiments, the at least 90% sequence identity is 97% sequence identity to 10-40 consecutive nucleotides. In embodiments, the at least 90% sequence identity is 98% sequence identity to 10-40 consecutive nucleotides. In embodiments, the at least 90% sequence identity is 99% sequence identity to 10-40 consecutive nucleotides. In embodiments, the at least 90% sequence identity is 100% sequence identity to 10-40 consecutive nucleotides.

In embodiments, the at least 90% sequence identity is 91% sequence identity to 11-40 consecutive nucleotides. In embodiments, the at least 90% sequence identity is 92% sequence identity to 11-40 consecutive nucleotides. In embodiments, the at least 90% sequence identity is 93% sequence identity to 11-40 consecutive nucleotides. In embodiments, the at least 90% sequence identity is 94% sequence identity to 11-40 consecutive nucleotides. In embodiments, the at least 90% sequence identity is 95% sequence identity to 11-40 consecutive nucleotides. In embodiments, the at least 90% sequence identity is 96% sequence identity to 11-40 consecutive nucleotides. In embodiments, the at least 90% sequence identity is 97% sequence identity to 11-40 consecutive nucleotides. In embodiments, the at least 90% sequence identity is 98% sequence identity to 11-40 consecutive nucleotides. In embodiments, the at least 90% sequence identity is 99% sequence identity to 11-40 consecutive nucleotides. In embodiments, the at least 90% sequence identity is 100% sequence identity to 11-40 consecutive nucleotides.

In embodiments, the at least 90% sequence identity is 91% sequence identity to 12-40 consecutive nucleotides. In embodiments, the at least 90% sequence identity is 92% sequence identity to 12-40 consecutive nucleotides. In embodiments, the at least 90% sequence identity is 93% sequence identity to 12-40 consecutive nucleotides. In embodiments, the at least 90% sequence identity is 94% sequence identity to 12-40 consecutive nucleotides. In embodiments, the at least 90% sequence identity is 95% sequence identity to 12-40 consecutive nucleotides. In embodiments, the at least 90% sequence identity is 96% sequence identity to 12-40 consecutive nucleotides. In embodiments, the at least 90% sequence identity is 97% sequence identity to 12-40 consecutive nucleotides. In embodiments, the at least 90% sequence identity is 98% sequence identity to 12-40 consecutive nucleotides. In embodiments, the at least 90% sequence identity is 99% sequence identity to 12-40 consecutive nucleotides. In embodiments, the at least 90% sequence identity is 100% sequence identity to 12-40 consecutive nucleotides.

In embodiments, the at least 90% sequence identity is 91% sequence identity to 13-consecutive nucleotides. In embodiments, the at least 90% sequence identity is 92% sequence identity to 13-40 consecutive nucleotides. In embodiments, the at least 90% sequence identity is 93% sequence identity to 13-40 consecutive nucleotides. In embodiments, the at least 90% sequence identity is 94% sequence identity to 13-40 consecutive nucleotides. In embodiments, the at least 90% sequence identity is 95% sequence identity to 13-40 consecutive nucleotides. In embodiments, the at least 90% sequence identity is 96% sequence identity to 13-40 consecutive nucleotides. In embodiments, the at least 90% sequence identity is 97% sequence identity to 13-40 consecutive nucleotides. In embodiments, the at least 90% sequence identity is 98% sequence identity to 13-40 consecutive nucleotides. In embodiments, the at least 90% sequence identity is 99% sequence identity to 13-40 consecutive nucleotides. In embodiments, the at least 90% sequence identity is 100% sequence identity to 13-40 consecutive nucleotides.

In embodiments, the at least 90% sequence identity is 91% sequence identity to 14-40 consecutive nucleotides. In embodiments, the at least 90% sequence identity is 92% sequence identity to 14-40 consecutive nucleotides. In embodiments, the at least 90% sequence identity is 93% sequence identity to 14-40 consecutive nucleotides. In embodiments, the at least 90% sequence identity is 94% sequence identity to 14-40 consecutive nucleotides. In embodiments, the at least 90% sequence identity is 95% sequence identity to 14-40 consecutive nucleotides. In embodiments, the at least 90% sequence identity is 96% sequence identity to 14-40 consecutive nucleotides. In embodiments, the at least 90% sequence identity is 97% sequence identity to 14-40 consecutive nucleotides. In embodiments, the at least 90% sequence identity is 98% sequence identity to 14-40 consecutive nucleotides. In embodiments, the at least 90% sequence identity is 99% sequence identity to 14-40 consecutive nucleotides. In embodiments, the at least 90% sequence identity is 100% sequence identity to 14-40 consecutive nucleotides.

In embodiments, the at least 90% sequence identity is 91% sequence identity to 15-40 consecutive nucleotides. In embodiments, the at least 90% sequence identity is 92% sequence identity to 15-40 consecutive nucleotides. In embodiments, the at least 90% sequence identity is 93% sequence identity to 15-40 consecutive nucleotides. In embodiments, the at least 90% sequence identity is 94% sequence identity to 15-40 consecutive nucleotides. In embodiments, the at least 90% sequence identity is 95% sequence identity to 15-40 consecutive nucleotides. In embodiments, the at least 90% sequence identity is 96% sequence identity to 15-40 consecutive nucleotides. In embodiments, the at least 90% sequence identity is 97% sequence identity to 15-40 consecutive nucleotides. In embodiments, the at least 90% sequence identity is 98% sequence identity to 15-40 consecutive nucleotides. In embodiments, the at least 90% sequence identity is 99% sequence identity to 15-40 consecutive nucleotides. In embodiments, the at least 90% sequence identity is 100% sequence identity to 15-40 consecutive nucleotides.

In embodiments, the at least 90% sequence identity is 91% sequence identity to 16-40 consecutive nucleotides. In embodiments, the at least 90% sequence identity is 92% sequence identity to 16-40 consecutive nucleotides. In embodiments, the at least 90% sequence identity is 93% sequence identity to 16-40 consecutive nucleotides. In embodiments, the at least 90% sequence identity is 94% sequence identity to 16-40 consecutive nucleotides. In embodiments, the at least 90% sequence identity is 95% sequence identity to 16-40 consecutive nucleotides. In embodiments, the at least 90% sequence identity is 96% sequence identity to 16-40 consecutive nucleotides. In embodiments, the at least 90% sequence identity is 97% sequence identity to 16-40 consecutive nucleotides. In embodiments, the at least 90% sequence identity is 98% sequence identity to 16-40 consecutive nucleotides. In embodiments, the at least 90% sequence identity is 99% sequence identity to 16-40 consecutive nucleotides. In embodiments, the at least 90% sequence identity is 100% sequence identity to 16-40 consecutive nucleotides.

In embodiments, the at least 90% sequence identity is 91% sequence identity to 17-40 consecutive nucleotides. In embodiments, the at least 90% sequence identity is 92% sequence identity to 17-40 consecutive nucleotides. In embodiments, the at least 90% sequence identity is 93% sequence identity to 17-40 consecutive nucleotides. In embodiments, the at least 90% sequence identity is 94% sequence identity to 17-40 consecutive nucleotides. In embodiments, the at least 90% sequence identity is 95% sequence identity to 17-40 consecutive nucleotides. In embodiments, the at least 90% sequence identity is 96% sequence identity to 17-40 consecutive nucleotides. In embodiments, the at least 90% sequence identity is 97% sequence identity to 17-40 consecutive nucleotides. In embodiments, the at least 90% sequence identity is 98% sequence identity to 17-40 consecutive nucleotides. In embodiments, the at least 90% sequence identity is 99% sequence identity to 17-40 consecutive nucleotides. In embodiments, the at least 90% sequence identity is 100% sequence identity to 17-40 consecutive nucleotides.

In embodiments, the at least 90% sequence identity is 91% sequence identity to 18-40 consecutive nucleotides. In embodiments, the at least 90% sequence identity is 92% sequence identity to 18-40 consecutive nucleotides. In embodiments, the at least 90% sequence identity is 93% sequence identity to 18-40 consecutive nucleotides. In embodiments, the at least 90% sequence identity is 94% sequence identity to 18-40 consecutive nucleotides. In embodiments, the at least 90% sequence identity is 95% sequence identity to 18-40 consecutive nucleotides. In embodiments, the at least 90% sequence identity is 96% sequence identity to 18-40 consecutive nucleotides. In embodiments, the at least 90% sequence identity is 97% sequence identity to 18-40 consecutive nucleotides. In embodiments, the at least 90% sequence identity is 98% sequence identity to 18-40 consecutive nucleotides. In embodiments, the at least 90% sequence identity is 99% sequence identity to 18-40 consecutive nucleotides. In embodiments, the at least 90% sequence identity is 100% sequence identity to 18-40 consecutive nucleotides.

In embodiments, the at least 90% sequence identity is 91% sequence identity to 19-40 consecutive nucleotides. In embodiments, the at least 90% sequence identity is 92% sequence identity to 19-40 consecutive nucleotides. In embodiments, the at least 90% sequence identity is 93% sequence identity to 19-40 consecutive nucleotides. In embodiments, the at least 90% sequence identity is 94% sequence identity to 19-40 consecutive nucleotides. In embodiments, the at least 90% sequence identity is 95% sequence identity to 19-40 consecutive nucleotides. In embodiments, the at least 90% sequence identity is 96% sequence identity to 19-40 consecutive nucleotides. In embodiments, the at least 90% sequence identity is 97% sequence identity to 19-40 consecutive nucleotides. In embodiments, the at least 90% sequence identity is 98% sequence identity to 19-40 consecutive nucleotides. In embodiments, the at least 90% sequence identity is 99% sequence identity to 19-40 consecutive nucleotides. In embodiments, the at least 90% sequence identity is 100% sequence identity to 19-40 consecutive nucleotides.

In embodiments, the at least 90% sequence identity is 91% sequence identity to 20-40 consecutive nucleotides. In embodiments, the at least 90% sequence identity is 92% sequence identity to 20-40 consecutive nucleotides. In embodiments, the at least 90% sequence identity is 93% sequence identity to 20-40 consecutive nucleotides. In embodiments, the at least 90% sequence identity is 94% sequence identity to 20-40 consecutive nucleotides. In embodiments, the at least 90% sequence identity is 95% sequence identity to 20-40 consecutive nucleotides. In embodiments, the at least 90% sequence identity is 96% sequence identity to 20-40 consecutive nucleotides. In embodiments, the at least 90% sequence identity is 97% sequence identity to 20-40 consecutive nucleotides. In embodiments, the at least 90% sequence identity is 98% sequence identity to 20-40 consecutive nucleotides. In embodiments, the at least 90% sequence identity is 99% sequence identity to 20-40 consecutive nucleotides. In embodiments, the at least 90% sequence identity is 100% sequence identity to 20-40 consecutive nucleotides.

In embodiments, the at least 90% sequence identity is 91% sequence identity to 25-40 consecutive nucleotides. In embodiments, the at least 90% sequence identity is 92% sequence identity to 25-40 consecutive nucleotides. In embodiments, the at least 90% sequence identity is 93% sequence identity to 25-40 consecutive nucleotides. In embodiments, the at least 90% sequence identity is 94% sequence identity to 25-40 consecutive nucleotides. In embodiments, the at least 90% sequence identity is 95% sequence identity to 25-40 consecutive nucleotides. In embodiments, the at least 90% sequence identity is 96% sequence identity to 25-40 consecutive nucleotides. In embodiments, the at least 90% sequence identity is 97% sequence identity to 25-40 consecutive nucleotides. In embodiments, the at least 90% sequence identity is 98% sequence identity to 25-40 consecutive nucleotides. In embodiments, the at least 90% sequence identity is 99% sequence identity to 25-40 consecutive nucleotides. In embodiments, the at least 90% sequence identity is 100% sequence identity to 25-40 consecutive nucleotides.

In embodiments, the at least 90% sequence identity is 91% sequence identity to 30-40 consecutive nucleotides. In embodiments, the at least 90% sequence identity is 92% sequence identity to 30-40 consecutive nucleotides. In embodiments, the at least 90% sequence identity is 93% sequence identity to 30-40 consecutive nucleotides. In embodiments, the at least 90% sequence identity is 94% sequence identity to 30-40 consecutive nucleotides. In embodiments, the at least 90% sequence identity is 95% sequence identity to 30-40 consecutive nucleotides. In embodiments, the at least 90% sequence identity is 96% sequence identity to 30-40 consecutive nucleotides. In embodiments, the at least 90% sequence identity is 97% sequence identity to 30-40 consecutive nucleotides. In embodiments, the at least 90% sequence identity is 98% sequence identity to 30-40 consecutive nucleotides. In embodiments, the at least 90% sequence identity is 99% sequence identity to 30-40 consecutive nucleotides. In embodiments, the at least 90% sequence identity is 100% sequence identity to 30-40 consecutive nucleotides.

In embodiments, the at least 90% sequence identity is 91% sequence identity to 35-40 consecutive nucleotides. In embodiments, the at least 90% sequence identity is 92% sequence identity to 35-40 consecutive nucleotides. In embodiments, the at least 90% sequence identity is 93% sequence identity to 35-40 consecutive nucleotides. In embodiments, the at least 90% sequence identity is 94% sequence identity to 35-40 consecutive nucleotides. In embodiments, the at least 90% sequence identity is 95% sequence identity to 35-40 consecutive nucleotides. In embodiments, the at least 90% sequence identity is 96% sequence identity to 35-40 consecutive nucleotides. In embodiments, the at least 90% sequence identity is 97% sequence identity to 35-40 consecutive nucleotides. In embodiments, the at least 90% sequence identity is 98% sequence identity to 35-40 consecutive nucleotides. In embodiments, the at least 90% sequence identity is 99% sequence identity to 35-40 consecutive nucleotides. In embodiments, the at least 90% sequence identity is 100% sequence identity to 35-40 consecutive nucleotides.

In embodiments, the at least 90% sequence identity is 91% sequence identity to 10-30 consecutive nucleotides. In embodiments, the at least 90% sequence identity is 92% sequence identity to 10-30 consecutive nucleotides. In embodiments, the at least 90% sequence identity is 93% sequence identity to 10-30 consecutive nucleotides. In embodiments, the at least 90% sequence identity is 94% sequence identity to 10-30 consecutive nucleotides. In embodiments, the at least 90% sequence identity is 95% sequence identity to 10-30 consecutive nucleotides. In embodiments, the at least 90% sequence identity is 96% sequence identity to 10-30 consecutive nucleotides. In embodiments, the at least 90% sequence identity is 97% sequence identity to 10-30 consecutive nucleotides. In embodiments, the at least 90% sequence identity is 98% sequence identity to 10-30 consecutive nucleotides. In embodiments, the at least 90% sequence identity is 99% sequence identity to 10-30 consecutive nucleotides. In embodiments, the at least 90% sequence identity is 100% sequence identity to 10-30 consecutive nucleotides.

In embodiments, the at least 90% sequence identity is 91% sequence identity to 11-30 consecutive nucleotides. In embodiments, the at least 90% sequence identity is 92% sequence identity to 11-30 consecutive nucleotides. In embodiments, the at least 90% sequence identity is 93% sequence identity to 11-30 consecutive nucleotides. In embodiments, the at least 90% sequence identity is 94% sequence identity to 11-30 consecutive nucleotides. In embodiments, the at least 90% sequence identity is 95% sequence identity to 11-30 consecutive nucleotides. In embodiments, the at least 90% sequence identity is 96% sequence identity to 11-30 consecutive nucleotides. In embodiments, the at least 90% sequence identity is 97% sequence identity to 11-30 consecutive nucleotides. In embodiments, the at least 90% sequence identity is 98% sequence identity to 11-30 consecutive nucleotides. In embodiments, the at least 90% sequence identity is 99% sequence identity to 11-30 consecutive nucleotides. In embodiments, the at least 90% sequence identity is 100% sequence identity to 11-30 consecutive nucleotides.

In embodiments, the at least 90% sequence identity is 91% sequence identity to 12-30 consecutive nucleotides. In embodiments, the at least 90% sequence identity is 92% sequence identity to 12-30 consecutive nucleotides. In embodiments, the at least 90% sequence identity is 93% sequence identity to 12-30 consecutive nucleotides. In embodiments, the at least 90% sequence identity is 94% sequence identity to 12-30 consecutive nucleotides. In embodiments, the at least 90% sequence identity is 95% sequence identity to 12-30 consecutive nucleotides. In embodiments, the at least 90% sequence identity is 96% sequence identity to 12-30 consecutive nucleotides. In embodiments, the at least 90% sequence identity is 97% sequence identity to 12-30 consecutive nucleotides. In embodiments, the at least 90% sequence identity is 98% sequence identity to 12-30 consecutive nucleotides. In embodiments, the at least 90% sequence identity is 99% sequence identity to 12-30 consecutive nucleotides. In embodiments, the at least 90% sequence identity is 100% sequence identity to 12-30 consecutive nucleotides.

In embodiments, the at least 90% sequence identity is 91% sequence identity to 13-30 consecutive nucleotides. In embodiments, the at least 90% sequence identity is 92% sequence identity to 13-30 consecutive nucleotides. In embodiments, the at least 90% sequence identity is 93% sequence identity to 13-30 consecutive nucleotides. In embodiments, the at least 90% sequence identity is 94% sequence identity to 13-30 consecutive nucleotides. In embodiments, the at least 90% sequence identity is 95% sequence identity to 13-30 consecutive nucleotides. In embodiments, the at least 90% sequence identity is 96% sequence identity to 13-30 consecutive nucleotides. In embodiments, the at least 90% sequence identity is 97% sequence identity to 13-30 consecutive nucleotides. In embodiments, the at least 90% sequence identity is 98% sequence identity to 13-30 consecutive nucleotides. In embodiments, the at least 90% sequence identity is 99% sequence identity to 13-30 consecutive nucleotides. In embodiments, the at least 90% sequence identity is 100% sequence identity to 13-30 consecutive nucleotides.

In embodiments, the at least 90% sequence identity is 91% sequence identity to 14-30 consecutive nucleotides. In embodiments, the at least 90% sequence identity is 92% sequence identity to 14-30 consecutive nucleotides. In embodiments, the at least 90% sequence identity is 93% sequence identity to 14-30 consecutive nucleotides. In embodiments, the at least 90% sequence identity is 94% sequence identity to 14-30 consecutive nucleotides. In embodiments, the at least 90% sequence identity is 95% sequence identity to 14-30 consecutive nucleotides. In embodiments, the at least 90% sequence identity is 96% sequence identity to 14-30 consecutive nucleotides. In embodiments, the at least 90% sequence identity is 97% sequence identity to 14-30 consecutive nucleotides. In embodiments, the at least 90% sequence identity is 98% sequence identity to 14-30 consecutive nucleotides. In embodiments, the at least 90% sequence identity is 99% sequence identity to 14-30 consecutive nucleotides. In embodiments, the at least 90% sequence identity is 100% sequence identity to 14-30 consecutive nucleotides.

In embodiments, the at least 90% sequence identity is 91% sequence identity to 15-30 consecutive nucleotides. In embodiments, the at least 90% sequence identity is 92% sequence identity to 15-30 consecutive nucleotides. In embodiments, the at least 90% sequence identity is 93% sequence identity to 15-30 consecutive nucleotides. In embodiments, the at least 90% sequence identity is 94% sequence identity to 15-30 consecutive nucleotides. In embodiments, the at least 90% sequence identity is 95% sequence identity to 15-30 consecutive nucleotides. In embodiments, the at least 90% sequence identity is 96% sequence identity to 15-30 consecutive nucleotides. In embodiments, the at least 90% sequence identity is 97% sequence identity to 15-30 consecutive nucleotides. In embodiments, the at least 90% sequence identity is 98% sequence identity to 15-30 consecutive nucleotides. In embodiments, the at least 90% sequence identity is 99% sequence identity to 15-30 consecutive nucleotides. In embodiments, the at least 90% sequence identity is 100% sequence identity to 15-30 consecutive nucleotides.

In embodiments, the at least 90% sequence identity is 91% sequence identity to 16-30 consecutive nucleotides. In embodiments, the at least 90% sequence identity is 92% sequence identity to 16-30 consecutive nucleotides. In embodiments, the at least 90% sequence identity is 93% sequence identity to 16-30 consecutive nucleotides. In embodiments, the at least 90% sequence identity is 94% sequence identity to 16-30 consecutive nucleotides. In embodiments, the at least 90% sequence identity is 95% sequence identity to 16-30 consecutive nucleotides. In embodiments, the at least 90% sequence identity is 96% sequence identity to 16-30 consecutive nucleotides. In embodiments, the at least 90% sequence identity is 97% sequence identity to 16-30 consecutive nucleotides. In embodiments, the at least 90% sequence identity is 98% sequence identity to 16-30 consecutive nucleotides. In embodiments, the at least 90% sequence identity is 99% sequence identity to 16-30 consecutive nucleotides. In embodiments, the at least 90% sequence identity is 100% sequence identity to 16-30 consecutive nucleotides.

In embodiments, the at least 90% sequence identity is 91% sequence identity to 17-30 consecutive nucleotides. In embodiments, the at least 90% sequence identity is 92% sequence identity to 17-30 consecutive nucleotides. In embodiments, the at least 90% sequence identity is 93% sequence identity to 17-30 consecutive nucleotides. In embodiments, the at least 90% sequence identity is 94% sequence identity to 17-30 consecutive nucleotides. In embodiments, the at least 90% sequence identity is 95% sequence identity to 17-30 consecutive nucleotides. In embodiments, the at least 90% sequence identity is 96% sequence identity to 17-30 consecutive nucleotides. In embodiments, the at least 90% sequence identity is 97% sequence identity to 17-30 consecutive nucleotides. In embodiments, the at least 90% sequence identity is 98% sequence identity to 17-30 consecutive nucleotides. In embodiments, the at least 90% sequence identity is 99% sequence identity to 17-30 consecutive nucleotides. In embodiments, the at least 90% sequence identity is 100% sequence identity to 17-30 consecutive nucleotides.

In embodiments, the at least 90% sequence identity is 91% sequence identity to 18-30 consecutive nucleotides. In embodiments, the at least 90% sequence identity is 92% sequence identity to 18-30 consecutive nucleotides. In embodiments, the at least 90% sequence identity is 93% sequence identity to 18-30 consecutive nucleotides. In embodiments, the at least 90% sequence identity is 94% sequence identity to 18-30 consecutive nucleotides. In embodiments, the at least 90% sequence identity is 95% sequence identity to 18-30 consecutive nucleotides. In embodiments, the at least 90% sequence identity is 96% sequence identity to 18-30 consecutive nucleotides. In embodiments, the at least 90% sequence identity is 97% sequence identity to 18-30 consecutive nucleotides. In embodiments, the at least 90% sequence identity is 98% sequence identity to 18-30 consecutive nucleotides. In embodiments, the at least 90% sequence identity is 99% sequence identity to 18-30 consecutive nucleotides. In embodiments, the at least 90% sequence identity is 100% sequence identity to 18-30 consecutive nucleotides.

In embodiments, the at least 90% sequence identity is 91% sequence identity to 19-30 consecutive nucleotides. In embodiments, the at least 90% sequence identity is 92% sequence identity to 19-30 consecutive nucleotides. In embodiments, the at least 90% sequence identity is 93% sequence identity to 19-30 consecutive nucleotides. In embodiments, the at least 90% sequence identity is 94% sequence identity to 19-30 consecutive nucleotides. In embodiments, the at least 90% sequence identity is 95% sequence identity to 19-30 consecutive nucleotides. In embodiments, the at least 90% sequence identity is 96% sequence identity to 19-30 consecutive nucleotides. In embodiments, the at least 90% sequence identity is 97% sequence identity to 19-30 consecutive nucleotides. In embodiments, the at least 90% sequence identity is 98% sequence identity to 19-30 consecutive nucleotides. In embodiments, the at least 90% sequence identity is 99% sequence identity to 19-30 consecutive nucleotides. In embodiments, the at least 90% sequence identity is 100% sequence identity to 19-30 consecutive nucleotides.

In embodiments, the at least 90% sequence identity is 91% sequence identity to 20-30 consecutive nucleotides. In embodiments, the at least 90% sequence identity is 92% sequence identity to 20-30 consecutive nucleotides. In embodiments, the at least 90% sequence identity is 93% sequence identity to 20-30 consecutive nucleotides. In embodiments, the at least 90% sequence identity is 94% sequence identity to 20-30 consecutive nucleotides. In embodiments, the at least 90% sequence identity is 95% sequence identity to 20-30 consecutive nucleotides. In embodiments, the at least 90% sequence identity is 96% sequence identity to 20-30 consecutive nucleotides. In embodiments, the at least 90% sequence identity is 97% sequence identity to 20-30 consecutive nucleotides. In embodiments, the at least 90% sequence identity is 98% sequence identity to 20-30 consecutive nucleotides. In embodiments, the at least 90% sequence identity is 99% sequence identity to 20-30 consecutive nucleotides. In embodiments, the at least 90% sequence identity is 100% sequence identity to 20-30 consecutive nucleotides.

In embodiments, the at least 90% sequence identity is 91% sequence identity to 10-20 consecutive nucleotides. In embodiments, the at least 90% sequence identity is 92% sequence identity to 10-20 consecutive nucleotides. In embodiments, the at least 90% sequence identity is 93% sequence identity to 10-20 consecutive nucleotides. In embodiments, the at least 90% sequence identity is 94% sequence identity to 10-20 consecutive nucleotides. In embodiments, the at least 90% sequence identity is 95% sequence identity to 10-20 consecutive nucleotides. In embodiments, the at least 90% sequence identity is 96% sequence identity to 10-20 consecutive nucleotides. In embodiments, the at least 90% sequence identity is 97% sequence identity to 10-20 consecutive nucleotides. In embodiments, the at least 90% sequence identity is 98% sequence identity to 10-20 consecutive nucleotides. In embodiments, the at least 90% sequence identity is 99% sequence identity to 10-20 consecutive nucleotides. In embodiments, the at least 90% sequence identity is 100% sequence identity to 10-20 consecutive nucleotides.

In embodiments, the at least 90% sequence identity is 91% sequence identity to 11-20 consecutive nucleotides. In embodiments, the at least 90% sequence identity is 92% sequence identity to 11-20 consecutive nucleotides. In embodiments, the at least 90% sequence identity is 93% sequence identity to 11-20 consecutive nucleotides. In embodiments, the at least 90% sequence identity is 94% sequence identity to 11-20 consecutive nucleotides. In embodiments, the at least 90% sequence identity is 95% sequence identity to 11-20 consecutive nucleotides. In embodiments, the at least 90% sequence identity is 96% sequence identity to 11-20 consecutive nucleotides. In embodiments, the at least 90% sequence identity is 97% sequence identity to 11-20 consecutive nucleotides. In embodiments, the at least 90% sequence identity is 98% sequence identity to 11-20 consecutive nucleotides. In embodiments, the at least 90% sequence identity is 99% sequence identity to 11-20 consecutive nucleotides. In embodiments, the at least 90% sequence identity is 100% sequence identity to 11-20 consecutive nucleotides.

In embodiments, the at least 90% sequence identity is 91% sequence identity to 12-20 consecutive nucleotides. In embodiments, the at least 90% sequence identity is 92% sequence identity to 12-20 consecutive nucleotides. In embodiments, the at least 90% sequence identity is 93% sequence identity to 12-20 consecutive nucleotides. In embodiments, the at least 90% sequence identity is 94% sequence identity to 12-20 consecutive nucleotides. In embodiments, the at least 90% sequence identity is 95% sequence identity to 12-20 consecutive nucleotides. In embodiments, the at least 90% sequence identity is 96% sequence identity to 12-20 consecutive nucleotides. In embodiments, the at least 90% sequence identity is 97% sequence identity to 12-20 consecutive nucleotides. In embodiments, the at least 90% sequence identity is 98% sequence identity to 12-20 consecutive nucleotides. In embodiments, the at least 90% sequence identity is 99% sequence identity to 12-20 consecutive nucleotides. In embodiments, the at least 90% sequence identity is 100% sequence identity to 12-20 consecutive nucleotides.

In embodiments, the at least 90% sequence identity is 91% sequence identity to 13-20 consecutive nucleotides. In embodiments, the at least 90% sequence identity is 92% sequence identity to 13-20 consecutive nucleotides. In embodiments, the at least 90% sequence identity is 93% sequence identity to 13-20 consecutive nucleotides. In embodiments, the at least 90% sequence identity is 94% sequence identity to 13-20 consecutive nucleotides. In embodiments, the at least 90% sequence identity is 95% sequence identity to 13-20 consecutive nucleotides. In embodiments, the at least 90% sequence identity is 96% sequence identity to 13-20 consecutive nucleotides. In embodiments, the at least 90% sequence identity is 97% sequence identity to 13-20 consecutive nucleotides. In embodiments, the at least 90% sequence identity is 98% sequence identity to 13-20 consecutive nucleotides. In embodiments, the at least 90% sequence identity is 99% sequence identity to 13-20 consecutive nucleotides. In embodiments, the at least 90% sequence identity is 100% sequence identity to 13-20 consecutive nucleotides.

In embodiments, the at least 90% sequence identity is 91% sequence identity to 14-20 consecutive nucleotides. In embodiments, the at least 90% sequence identity is 92% sequence identity to 14-20 consecutive nucleotides. In embodiments, the at least 90% sequence identity is 93% sequence identity to 14-20 consecutive nucleotides. In embodiments, the at least 90% sequence identity is 94% sequence identity to 14-20 consecutive nucleotides. In embodiments, the at least 90% sequence identity is 95% sequence identity to 14-20 consecutive nucleotides. In embodiments, the at least 90% sequence identity is 96% sequence identity to 14-20 consecutive nucleotides. In embodiments, the at least 90% sequence identity is 97% sequence identity to 14-20 consecutive nucleotides. In embodiments, the at least 90% sequence identity is 98% sequence identity to 14-20 consecutive nucleotides. In embodiments, the at least 90% sequence identity is 99% sequence identity to 14-20 consecutive nucleotides. In embodiments, the at least 90% sequence identity is 100% sequence identity to 14-20 consecutive nucleotides.

In embodiments, the at least 90% sequence identity is 91% sequence identity to 15-20 consecutive nucleotides. In embodiments, the at least 90% sequence identity is 92% sequence identity to 15-20 consecutive nucleotides. In embodiments, the at least 90% sequence identity is 93% sequence identity to 15-20 consecutive nucleotides. In embodiments, the at least 90% sequence identity is 94% sequence identity to 15-20 consecutive nucleotides. In embodiments, the at least 90% sequence identity is 95% sequence identity to 15-20 consecutive nucleotides. In embodiments, the at least 90% sequence identity is 96% sequence identity to 15-20 consecutive nucleotides. In embodiments, the at least 90% sequence identity is 97% sequence identity to 15-20 consecutive nucleotides. In embodiments, the at least 90% sequence identity is 98% sequence identity to 15-20 consecutive nucleotides. In embodiments, the at least 90% sequence identity is 99% sequence identity to 15-20 consecutive nucleotides. In embodiments, the at least 90% sequence identity is 100% sequence identity to 15-20 consecutive nucleotides.

In embodiments, the at least 90% sequence identity is 91% sequence identity to 16-20 consecutive nucleotides. In embodiments, the at least 90% sequence identity is 92% sequence identity to 16-20 consecutive nucleotides. In embodiments, the at least 90% sequence identity is 93% sequence identity to 16-20 consecutive nucleotides. In embodiments, the at least 90% sequence identity is 94% sequence identity to 16-20 consecutive nucleotides. In embodiments, the at least 90% sequence identity is 95% sequence identity to 16-20 consecutive nucleotides. In embodiments, the at least 90% sequence identity is 96% sequence identity to 16-20 consecutive nucleotides. In embodiments, the at least 90% sequence identity is 97% sequence identity to 16-20 consecutive nucleotides. In embodiments, the at least 90% sequence identity is 98% sequence identity to 16-20 consecutive nucleotides. In embodiments, the at least 90% sequence identity is 99% sequence identity to 16-20 consecutive nucleotides. In embodiments, the at least 90% sequence identity is 100% sequence identity to 16-20 consecutive nucleotides.

In embodiments, the at least 90% sequence identity is 91% sequence identity to 17-20 consecutive nucleotides. In embodiments, the at least 90% sequence identity is 92% sequence identity to 17-20 consecutive nucleotides. In embodiments, the at least 90% sequence identity is 93% sequence identity to 17-20 consecutive nucleotides. In embodiments, the at least 90% sequence identity is 94% sequence identity to 17-20 consecutive nucleotides. In embodiments, the at least 90% sequence identity is 95% sequence identity to 17-20 consecutive nucleotides. In embodiments, the at least 90% sequence identity is 96% sequence identity to 17-20 consecutive nucleotides. In embodiments, the at least 90% sequence identity is 97% sequence identity to 17-20 consecutive nucleotides. In embodiments, the at least 90% sequence identity is 98% sequence identity to 17-20 consecutive nucleotides. In embodiments, the at least 90% sequence identity is 99% sequence identity to 17-20 consecutive nucleotides. In embodiments, the at least 90% sequence identity is 100% sequence identity to 17-20 consecutive nucleotides.

In embodiments, the at least 90% sequence identity is 91% sequence identity to 18-20 consecutive nucleotides. In embodiments, the at least 90% sequence identity is 92% sequence identity to 18-20 consecutive nucleotides. In embodiments, the at least 90% sequence identity is 93% sequence identity to 18-20 consecutive nucleotides. In embodiments, the at least 90% sequence identity is 94% sequence identity to 18-20 consecutive nucleotides. In embodiments, the at least 90% sequence identity is 95% sequence identity to 18-20 consecutive nucleotides. In embodiments, the at least 90% sequence identity is 96% sequence identity to 18-20 consecutive nucleotides. In embodiments, the at least 90% sequence identity is 97% sequence identity to 18-20 consecutive nucleotides. In embodiments, the at least 90% sequence identity is 98% sequence identity to 18-20 consecutive nucleotides. In embodiments, the at least 90% sequence identity is 99% sequence identity to 18-20 consecutive nucleotides. In embodiments, the at least 90% sequence identity is 100% sequence identity to 18-20 consecutive nucleotides.

In embodiments, the at least 90% sequence identity is 91% sequence identity to 19-20 consecutive nucleotides. In embodiments, the at least 90% sequence identity is 92% sequence identity to 19-20 consecutive nucleotides. In embodiments, the at least 90% sequence identity is 93% sequence identity to 19-20 consecutive nucleotides. In embodiments, the at least 90% sequence identity is 94% sequence identity to 19-20 consecutive nucleotides. In embodiments, the at least 90% sequence identity is 95% sequence identity to 19-20 consecutive nucleotides. In embodiments, the at least 90% sequence identity is 96% sequence identity to 19-20 consecutive nucleotides. In embodiments, the at least 90% sequence identity is 97% sequence identity to 19-20 consecutive nucleotides. In embodiments, the at least 90% sequence identity is 98% sequence identity to 19-20 consecutive nucleotides. In embodiments, the at least 90% sequence identity is 99% sequence identity to 19-20 consecutive nucleotides. In embodiments, the at least 90% sequence identity is 100% sequence identity to 19-20 consecutive nucleotides.

In embodiments, the at least 90% sequence identity is 91% sequence identity to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41 consecutive nucleotides. In embodiments, the at least 90% sequence identity is 92% sequence identity to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41 consecutive nucleotides. In embodiments, the at least 90% sequence identity is 93% sequence identity to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41 consecutive nucleotides. In embodiments, the at least 90% sequence identity is 94% sequence identity to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41 consecutive nucleotides. In embodiments, the at least 90% sequence identity is 95% sequence identity to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41 consecutive nucleotides. In embodiments, the at least 90% sequence identity is 96% sequence identity to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41 consecutive nucleotides. In embodiments, the at least 90% sequence identity is 97% sequence identity to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41 consecutive nucleotides. In embodiments, the at least 90% sequence identity is 98% sequence identity to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41 consecutive nucleotides. In embodiments, the at least 90% sequence identity is 99% sequence identity to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41 consecutive nucleotides. In embodiments, the at least 90% sequence identity is 100% sequence identity to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41 consecutive nucleotides.

### EXAMPLES

We have invented novel reagents for the rapid, sensitive, and specific detection of the novel coronavirus (SARS-CoV-2) using an isothermal nucleic acid amplification technique. These reagents entail novel oligo DNA primer sets for loop mediated amplification (LAMP) of SARS-CoV-2 RNA. The primer sets were designed such that they provide very efficient LAMP amplification specific for the pathogen. They will not amplify human DNA/RNA or those of other coronaviruses. We have tested our novel primer sets with commercial RNA standards for SARS-CoV-2, on saliva samples from COVID-19 patients, as well as on saliva from COVID-19 negative subjects. Our primer sets amplify regions of ORF1/2, the spike protein gene, and the nucleocapsid. Furthermore, one of the primer sets will only amplify RNA from a virus-positive sample, but not from the commercial standard. This, permits exclusion of false positive signals that could occur from potential contamination with a positive control or a standard. Attached manuscript draft contains detailed information on the primer sets and preliminary results. Our novel LAMP primer sets permits detection of the novel coronavirus SARS-CoV-2 in less than 10 minutes with sensitivity comparable to that of PCR.

Saliva was recently shown as a promising alternative specimen for COVID-19 diagnostics. In contrast to nasopharyngeal swabs, the collection of saliva does not pose the risk of healthcare worker exposure nor does it require scarce sampling materials. Furthermore, since transmission of the novel coronavirus involves droplets originating mostly from the upper respiratory tract, the viral load in saliva may be a very good indicator of the contagiousness of an infected person. To establish a rapid saliva-based diagnostic test, we designed and analyzed 17 novel sets of primers for isothermal loop-mediated amplification (LAMP) of several regions of the SARS-CoV-2 genome. Predicted internal hairpin structure formation and unfavorable melting temperatures were avoided. Primer sets with low homology to sequences of the human genome and those of other known human coronaviruses were chosen for synthesis and tested in vitro. We tested the performance of six primer sets in LAMP assays of saliva samples from known COVID-19 positive and negative individuals, as well as with commercial SARS-CoV-2 RNA standards. Our LAMP assays had sensitivities comparable to that of real time PCR, but provide definitive results faster. Average threshold times of 9.8±2.5 minutes were obtained with saliva samples from COVID-19 positive patients.

### Example 1: Introduction

In December of 2019, a respiratory illness caused by a novel coronavirus emerged in Wuhan, China. Because of its similarities to severe acute respiratory syndrome coronavirus (SARS-CoV), the novel coronavirus was termed SARS-CoV-2 and the disease that it causes has become known as COVID-19. As of Aug. 8, 2020, more than 19 million laboratory-confirmed cases of COVID-19 have been reported worldwide, including 716,075 deaths (1) and the WHO has declared the COVID-19 pandemic a “Public Health Emergency of International Concern” (2). While numerous COVID-19 therapeutics and vaccines are developed and tested, none are currently available for widespread use (3, 4). Thus, rapid, accurate, safe, and inexpensive diagnostic assays are currently play a vital role in curtailing the further spread of SARS-CoV-2. In fact, if COVID-19 testing can be performed rapidly and on-site, similar to a point-of-care (POC) approach, widespread test setups could be installed for screening purposes in many public or private places that deal with large crowds of people, including airports, train stations, ports, movie theaters, etc., a faster socio economic recovery phase could be achieved.

Most COVID-19 diagnostic assays utilize nasopharyngeal swabs (NPS) that are sent to centralized laboratories for testing by quantitative reverse-transcription polymerase chain reaction (qRT-PCR) (5, 6). NPS specimens must be collected using swabs made of synthetic material (e.g., polyester or nylon) with long plastic shafts, and the ongoing pandemic has led to swab shortages (7). Furthermore, NPS sample collection is an invasive process, which can cause discomfort, bleeding, sneezing and other sampling symptoms (8). During the collection process, trained healthcare personnel must come into close contact with patients, all of which increase the risk of transmission of the virus. Thus, alternative specimens for SARS-CoV-2 detection have been explored, including bronchoalveolar lavage fluid, sputum, saliva, serum, feces and urine (9, 10). In testing for respiratory viruses, To et al. demonstrated that saliva had a concordance rate of greater than 90% with nasopharyngeal swabs (11, 12) and that the median SARS-CoV-2 viral load in hospitalized COVID-19 patients was 3.3×10 6 copies/mL (13). In addition, collection of saliva specimens is a non-invasive procedure that can be performed without healthcare personnel. This minimizes the chance of healthcare workers being exposed to SARS-CoV-2 while also eliminating sample collection wait times.

Diagnostic assays designed to detect respiratory pathogens routinely employ qRT-PCR because it produces rapid results with high sensitivity and specificity (14, 15). However, there are several drawbacks to this method: it requires expensive equipment and trained personnel. To reduce equipment-related expenses, researchers have turned to isothermal amplification methods. One such method, loop-mediated amplification (LAMP) employs a set of 4-6 primers and a thermostable Bst polymerase for the isothermal amplification of DNA at a constant temperature (16, 17). In addition to polymerase and reverse transcriptase activity, Bst polymerase also possesses strand-displacement activity, which allows the forward and backward inner primers (FIP and BIP) to initiate invasion of the target strand. Following FIP-and-BIP-mediated extension, the F3 and B3 primers displace the single-stranded FIP/BIP product, which forms a self-hybridizing dumbbell-shaped loop structure. The loop structure contains multiple sites for initiation, which leads to the formation of long concatemers. Loop Forward and Loop Backward (LF and LB) primers can also be included, which anneal to and extend the singe-stranded loop structures, increasing the efficiency of the amplification process leading to shorter amplification times Furthermore, LAMP assays are much more tolerant of inhibitors than typical PCR reactions, meaning that crude samples can often be used rather than extracted or purified DNA or RNA (18).

In this study, we designed and evaluated 17 novel candidate LAMP primer sets targeting the ORF1ab gene, the spike gene, or the nucleocapsid gene of SARS-CoV-2 in silico. Based on this evaluation, six primer sets were selected for further optimization and evaluation, including testing with extracted viral RNA from the saliva of six patients who had been previously diagnosed with COVID-19 by qRT-PCR, and whose saliva tested either positive or negative for SARS-CoV-2, depending on whether the patient still had the disease or already recovered.

### Example 2: Materials and Methods

### Saliva Samples

Saliva samples were collected by qualified healthcare personnel from seven male City of Hope (COH) patients previously diagnosed with COVID-19 by COH's CLIA clinical lab using nasopharyngeal swabs. One mL saliva was collected from each patient using the OMNIgene ORAL Collection Device (DNAGenotek, Ottawa, Canada) according to the manufacturer's instructions. COVID-19 positive saliva samples were from three patients with reported onset of disease symptoms or were screened for COVID-19 upon hospitalization, within 4 days of saliva collection. Negative saliva samples were from four patients who were symptomatic 18 to 52 days before sample collection. All saliva samples were tested by RT-PCR and LAMP as described below.

Isolation of Viral RNA from Saliva

RNA extraction from saliva was performed using the QIAamp Viral RNA Mini Kit (Qiagen, Hilden, Germany) according to the manufacturer's “Spin Protocol”. Briefly, 140 μL, of buffered saliva was added to 550 μL, Buffer AVL and 10 μL, carrier RNA in a 1.5 mL RNAse-free microcentrifuge tube. The sample was vortexed, incubated for ten minutes at and 560 μL of 100% ethanol was added. The RNA was processed according to the manufacturer's instructions and then eluted from the column using 80 μL nuclease-free water.

qRT-PCR

Three primer-probe sets adapted from those designed or referenced by the National Reference Center for Respiratory Viruses, Institut Pasteur, Paris, France, and posted in a protocol by the World Health Organization (WHO) were used to perform real-time RT-PCR to detect SARS-CoV-2 viral RNA isolated from saliva samples were. Two sets targeted the SARS-CoV-2 RNA-dependent RNA polymerase (RdRP) gene (primers IP2 and IP4) and one set targeted the E gene (15, 19). Instead of black hole quenchers, we used internal ZEN and 3′ Iowa Black FQ quenchers offered by Integrated DNA technologies (IDT, Coralville, IA) that synthesized these oligonucleotides (Table 1).

qRT-PCR assays were performed using the SuperScript III Platinum One-Step qRT-PCR Kit (Invitrogen, Carlsbad, CA). Twenty-five microliters of reaction mix consisting of 2× reaction mix, 50× SuperScript III RT/Platinum Taq Mix, 10 μM each of primers/probes, mM MgSO₄, and 5 μL of extracted RNA. The E primer/probe set as used as a simplex mix and the IP2 and IP4 primer/probe sets were used as multiplex mixes. qRT-PCR was performed on a CFX96 Real-Time PCR Detection System (Bio-Rad Laboratories, Hercules, CA) with the following cycle parameters: 20 min at 55° C. for reverse transcription and 3 min of denaturation at 95° C. followed by 40 cycles of 15 seconds at 95° C. and 30 seconds at 58° C. The IP2 probe contained a hexachlorofluorescein (HEX) fluorophore and the IP4 and E probes were designed with fluorescein (FAM) fluorophores (Table 1). Accordingly, amplification signals were detected on the HEX and FAM channels. Commercially available in vitro SARS-CoV-2 RNA from Exact Diagnostics (EDX, Fort Worth, TX) was used as a positive control at a concentration of 1000 copies per reaction.

TABLE 1
Primer/probe sets for qRT-PCR detection of SARS-COV-2

| Target | | |
|---|---|---|
| Region | Name | Sequence |
| RdRP- | IP2 | ATGAGCTTAGTCCTGTTG |
| IP2 | Forward | |
| | IP2 | CTCCCTTTGTTGTGTTGT |
| | Reverse | |
| | IP2 Probe | /5HEX/AGATGTCTT/ZEN/GTGCTGCCGGTA/3IABKFQ/ |
| RdRP- | IP4 | GGTAACTGGTATGATTTCG |
| IP4 | Forward | |
| | IP4 | CTGGTCAAGGTTAATATAGG |
| | Reverse | |
| | IP4 Probe | /56-FAM/TCATACAAA/ZEN/CCACGCCAGG/3IABKFQ/ |
| E gene | E Forward | ACAGGTACGTTAATAGTTAATAGCGT |
| | E Reverse | ATATTGCAGCAGTACGCACACA |
| | E Probe | /56- |
| | | FAM/ACACTAGCC/ZEN/ATCCTTACTGCGCTTCG/3IABKFQ/ |

Legend:
5Hex = 5′-Hexachlorofluorescein; 56-Fam = 5′ 6-FAM (Fluorescein); IABKFQ = 3′ Iowa Black FQ; ZEN = IDT′s napthylene-azo compound (20).

### RT-LAMP Primer Design and Testing

Seventeen sets of LAMP primers were designed in silico (LAMP Designer Software, PREMIER Biosoft, Palo Alto, CA) to amplify three target regions within the SARS-CoV-2 genome: ORF1ab, the spike protein gene, and the nucleocapsid gene. Further analyses of each primer set were then performed using NCBI's Basic Local Alignment Search Tool (BLAST) for sequence specificity, the NextStrain database for diversity among SARS-CoV-2 strains, and Primer3 software for thermodynamic data. First, to determine if any homologous sequences were present in the human genome, NCBI BLASTn searches were performed using each primer in the set as a query sequence test for sequence similarity in the Homo sapiens (NCBI taxid:9606) genomes. To determine if any homologous sequences were present in non-SARS-CoV-2 human coronavirus genomes, NCBI BLASTn searches were performed using each primer in the set as a query sequence to identify sequence similarities in the genomes of the following human coronaviruses HCoV-SARS (taxid: 694009), MERS-CoV (taxid: 1335626), HCoV-229E (taxid: 11137), HCoV-HKU1 (taxid: 290028), HCoV-NL63 (taxid: 277944), HCoV-OC43 (taxid: 31631). Second, for the genomic position of each primer set, the highest diversity (entropy) score was determined for North-American strains of SARS-CoV-2 using the strain database on nextstrain.org (4485 genomes from North American strains were available when the site was accessed on 22 Jun. 2020). This “diversity score” is an indication of how much variability there is between strains at the regions of the genome where the primers bind. Third, a Primer3 python script was utilized to calculate hairpin, homodimer, and heterodimer formation thermodynamics. Primer3 software (21, 22) was used to determine the Gibbs free energy (ΔG) for each primer (hairpin and homodimer) or primer pair (heterodimer).

The selected oligonucleotide primer sets, as well as two sets of previously published LAMP primers (23), were custom synthesized by Integrated DNA Technologies (Table 2). LAMP assays were performed in a 20 μL reaction mixture containing: 1.6 μM each of FIP and BIP primers, 0.2 μM each of F3 and B3 primers, 0.4 μM each of LF and LB primers, 10 μL WarmStart LAMP 2× Master Mix (WarmStart LAMP Kit (DNA & RNA), New England BioLabs, Ipswich, MA), 0.15 μL fluorescent dye (WarmStart LAMP Kit (DNA & RNA), New England BioLabs, Ipswich, MA), the appropriate template RNA, and addition of nuclease-free water to 20 μL. The reaction mixture was heated at 65° C. for 63 minutes in a CFX96 Real-Time PCR Detection System (Bio-Rad Laboratories, Hercules, CA). Fluorescence measurements were taken every 30 seconds on the SYBR Green I/FAM channel (excitation: 450-490 nm, emission: 515-530 nm).

To verify their specificity, the six selected RT-LAMP primer sets as well as two previously published RT-LAMP primer sets were also tested against carrier RNA (QIAamp Viral RNA Mini Kit, Qiagen, Hilden, Germany) and human genomic DNA (SARS-CoV-2 Negative, Exact Diagnostics, Fort Worth, TX)).

TABLE 2
Novel primer sets designed for RT-LAMP detection of SARS-COV-2

| SEQ | Target | Primer | |
|---|---|---|---|
| ID NO: | Region | Name | Sequence |
| 1 | 5′-UTR/ | ORF2-F3 | AATCTGTGTGGCTGTCAC |
| 2 | ORF1ab | ORF2-B3 | TCTGATAAGACCTCCTCCAC |
| 3 | | ORF2-FIP | AGATGTGCTGATGATCGGCTGTGACAGGACACGAGTAACT |
| 4 | | ORF2-BIP | GTCCGGGTGTGACCGAAAGCTGAGTTGGACGTGTGTTT |
| 5 | | ORF2-LF | TAAGCAGCCTGCAGAAGATAG |
| 6 | | ORF2-LB | GTAAGATGGAGAGCCTTGTCC |
| 7 | ORF1ab | ORF3-F3 | CTTGGCACTGATCCTTATGAA |
| 8 | | ORF3-B3 | GTTCCGTGTACCAAGCAA |
| 9 | | ORF3-FIP | ATCGACATAGCGAGTGTATGCCAACATAGCAGTGGTGTTACC |
| 10 | | ORF3-BIP | GATGGCTACCCTCTTGAGTGCGTCCAGTTGTTCGGACAA |
| 11 | | ORF3-LF | AAGCTCACGCATGAGTTCA |
| 12 | | ORF3-LB | CGTGCTGGTAAAGCTTCATG |
| 13 | ORF1ab | ORF4-F3 | CAAGGTTACAAGAGTGTGAA |
| 14 | | ORF4-B3 | CTTCTTCTTCATCCTCATCTG |
| 15 | | ORF4-FIP | CACAACACAGGCGAACTCAGAGAAGTGCTCTGCCTATA |
| 16 | | ORF4-BIP | TTACTTACACCACTGGGCATTGAACTCACCAGACTCATCAA |
| 17 | | ORF4-LF | TTACTTCTGTACCGAGTTCAAC |
| 18 | | ORF4-LB | AGTGGAGTATGGCTACATACT |
| 19 | Spike | S11-F3 | CTTCTGCTAATCTTGCTGCTA |
| 20 | | S11-B3 | CATCACAGTTACCAGACACAA |
| 21 | | S11-FIP | TGCAGGGACATAAGTCACATGCGGCTATCATCTTATGTCCTTCC |
| 22 | | S11-BIP | TTCACAACTGCTCCTGCCATTGTGTGTGCCATTTGAAACAA |
| 23 | | S11-LF | CCATGAGGTGCTGACTGAG |
| 24 | | S11-LB | ACTTTCCTCGTGAAGGTGTC |
| 25 | Spike | S13-F3 | GGCTATCATCTTATGTCCTTCC |
| 26 | | S13-B3 | TACAACATCACAGTTACCAGAC |
| 27 | | S13-FIP | AATGGCAGGAGCAGTTGTGAACCTCATGGTGTAGTCTTCTTG |
| 28 | | S13-BIP | TGGAAAAGCACACTTTCCTCGTTGTGTTACAAACCAGTGTGT |
| 29 | | S13-LF | GTGCAGGGACATAAGTCACA |
| 30 | | S13-LB | AGGTGTCTTTGTTTCAAATGGC |
| 31 | Nucleocapsid | N17-F3 | TCAACATGGCAAGGAAGAC |
| 32 | | N17-B3 | TTGGTGTATTCAAGGCTCC |
| 33 | | N17-FIP | ACCGTCACCACCACGAATTCTTAACACCAATAGCAGTCCAG |
| 34 | | N17-BIP | CTAGGAACTGGGCCAGAAGCACCCATATGATGCCGTCT |
| 35 | | N17-LF | GCTCTTCGGTAGTAGCCAATT |
| 36 | | N17-LB | GACTTCCCTATGGTGCTAACAA |

Legend: Primer nomenclature (F3, B3, FIP, BIP, LF, and LB) as in ref. (16, 17)

### Dilution Series of SARS-CoV-2 Standard

The commercial RNA standard containing 2×10⁵ copies per mL of E, N, ORF1ab, RdRP, and S genes from SARS-CoV-2 was serially diluted in four-fold steps in SARS-CoV-2 Negative to 3.125×10³ copies to mL (i.e., approximately 3.9 copies per reaction). Five microliters of each dilution of the RNA was then used as a template for the RT-LAMP assays with primers sets ORF3, ORF4, S11, S13, and N17. Reactions with threshold times above 40 minutes were considered negative.

### Ethics Statement

This study was conducted in compliance with City of Hope's Institutional Review Board under approved protocol 20126.

### Example 3: Results

qRT-PCR

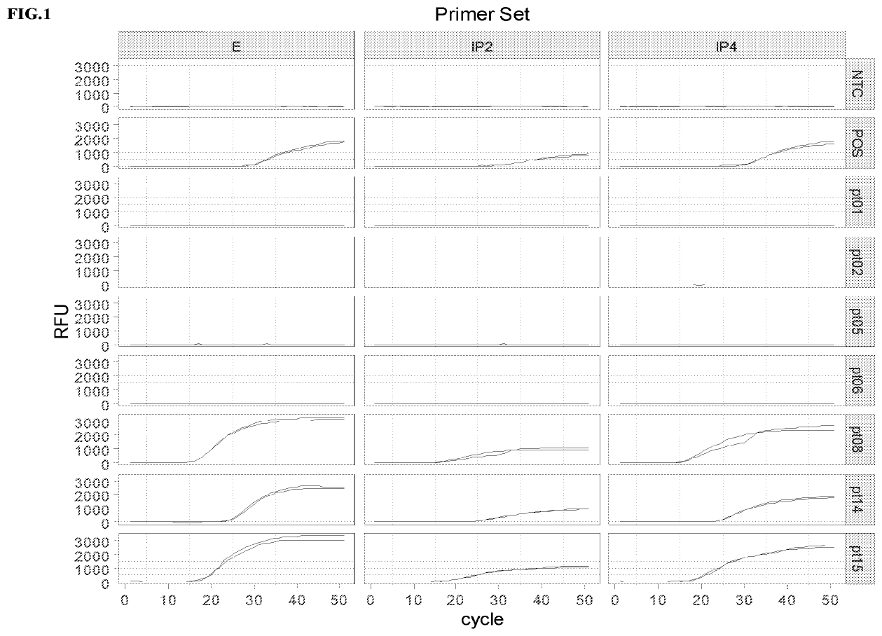

The seven patients included in this study had a positive nasopharyngeal swab qRT-PCR test in COH's CLIA lab (referred to as Test 1 in Table 3). Upon enrollment in the study, saliva samples were collected and qRT-PCR assays were performed to determine whether they were still positive for SARS-CoV-2 (referred to as Test 2 in Table 3). Four patients (1, 2, 5, and 6) tested negative and three patients (8, 14 and 15) tested positive for SARS-CoV-2 (Table 3, FIG. 1). These results served as a reference for the subsequent RT-LAMP assays.

TABLE 3
COVID-19 positive and negative saliva samples from patients
tested by RT-PCR (all male)

| | | symptom | days | | | | |
|---|---|---|---|---|---|---|---|
| | | onset before | between | | | | |
| | | positive NPS | Test 1 | | | | Result of |
| | | RT-PCR | (NPS) | | | | Test 2 |
| Patient | | (Test 1) | & Test 2 | Average Ct | | | (RT-PCR |
| # | Age | in days | (saliva) | E | IP2 | IP4 | on saliva) |
| 1 | 60 | 7 | 11 | — | — | — | Negative |
| 2 | 43 | 7 | 18 | — | — | — | Negative |
| 5 | 40 | 3 | 24 | — | — | — | Negative |
| 6 | 45 | 2 | 50 | — | — | — | Negative |
| 8 | 38 | 2 | 2 | 16.5 | 15.9 | 15.3 | Positive |
| 14 | 73 | 1 | 0 | 22.0 | 30.7 | 23.3 | Positive |
| 15 | ? | ? | ? | 18.4 | 17.4 | 18.2 | Positive |
| | | Positive control | | 29.5 | 30.7 | 30.0 | Positive |
| | | (1000 copies) | | | | | |
| | | | NTC | — | — | — | Negative |

Legend of Table 3: NPS, nasopharyngeal swab; -, no amplification observed; NTC, no template control; E, IP2, IP4 indicate primer sets. AS, patient was asymptomatic for COVID-19 when Test 1 was performed.

### LAMP Primer Design

Seventeen candidate LAMP primer sets were designed using the commercial software LAMP Designer (PREMIER Biosoft, Palo Alto, CA). All 17 candidate primer sets had a rating above 75 (Table 4) and thus were given quality ratings of “Best” by the software. To verify that the candidate primer sets would be specific for SARS-CoV-2, BLASTn was used to determine the homology of the primers to the sequences in the human genome and non-SARS-CoV-2 human coronavirus genomes (Table 4).

TABLE 4
In silico Evaluation of Candidate SARS-CoV-2 LAMP Primer Sets

| | | | | | BLAST | | | | | | |
|---|---|---|---|---|---|---|---|---|---|---|---|
| | | | BLAST | | (other human | | | | | | |
| | | | (Human) | | coronaviruses) | | | | | | |
| Primer | | | Avg. | | Avg. | | | | | | |
| Set | Region of | | Query | Avg. | Query | Avg. | Diversity | ΔG^{d} | | | |
| # | Genome | Ratingª | Coverage^{b} | Identity^{b} | Coverage^{b} | Identity^{b} | score^{c} | Hairpin | Homodimer | Heterodimer | Synthesized |
| 1 | ORF1ab | 90.8 | 71.2% | 99.2% | 88.7% | 98.8% | 0.133 | −2.2 | −3.9 | −5.6 | No |
| 2 | 5' UTR & | 89.5 | 73.5% | 100% | 83.3% | 98.0% | 0.010 | −1.3 | −6.7 | −6.2 | Yes |
| | ORF1ab | | | | | | | | | | |
| 3 | ORF1ab | 89 | 71.5% | 100% | 79.8% | 97.2% | 0.088 | −1.1 | −4.5 | −4.8 | Yes |
| 4 | ORF1ab | 92 | 71.3% | 100% | 78.8% | 96.4% | 0.028 | −2.4 | −4.0 | −6.1 | Yes |
| 5 | ORF1ab | 90.7 | 75.7% | 97.9% | 91.0% | 99.2% | 0.009 | 0.3 | −3.4 | −4.8 | No |
| 6 | ORF1ab | 85.4 | 81.0% | 98.4% | 77.0% | 98.5% | 0.046 | −1.4 | −4.9 | −5.5 | No |
| 7 | ORF1ab | 89.7 | 76.2% | 98.4% | 89.0% | 98.7% | 0.038 | −0.8 | −4.9 | −5.5 | No |
| 8 | ORF1ab | 88.9 | 82.5% | 98.3% | 88.7% | 96.5% | 0.007 | −1.6 | −4.6 | −4.7 | No |
| 9 | ORF1ab | 87.2 | 75.0% | 99.1% | 91.2% | 95.9% | 0.276 | −0.9 | −5.7 | −5.3 | No |
| 10 | ORF1ab | 85 | 89.0% | 99.1% | 78.8% | 95.8% | 0.007 | −1.0 | −6.2 | −5.3 | No |
| 11 | S | 89.4 | 83.7% | 98.2% | 81.7% | 97.6% | 0.009 | −2.5 | −10.4 | −4.9 | Yes |
| 12 | S | 87.5 | 83.7% | 99.2% | 84.0% | 100% | 0.560 | −3.4 | −6.5 | −6.1 | No |
| 13 | S | 87.5 | 86.2% | 98.3% | 83.7% | 99.2% | 0.009 | −0.7 | −3.2 | −7.1 | Yes |
| 14 | N | 92.6 | 84.5% | 99.1% | 93.0% | 99.1% | 0.319 | −1.3 | −7.9 | −6.0 | No |
| 15 | N | 92.1 | 88.8% | 98.2% | 89.3% | 98.3% | 0.319 | −0.3 | −5.1 | −8.7 | No |
| 16 | N | 90.1 | 85.8% | 98.3% | 91.7% | 96.2% | 0.319 | −0.9 | −5.8 | −7.0 | No |
| 17 | N | 89.2 | 79.2% | 98.3% | 90.2% | 98.4% | 0.061 | −0.9 | −10.8 | −6.0 | Yes |

^{a}Rating from LAMP Designer software.
^{b}Average of the highest query coverage or percent identity found in the BLAST results for each primer set.
^{c}Highest diversity (entropy) score for North-American strains of SARS-CoV-2 using the database on nextstrain.org.
^{d}Primer3 software was used to determine the Gibbs free energy (ΔG) in kcal/mol for each primer (hairpin and homodimer) or primer pair (heterodimer). The lowest ΔG for each primer set is listed.
Primer sets 2, 3, 4, 11, 13, and 17 are referred to as ORF2, ORF3, ORF4, S11, S13, and N17, respectively, elsewhere in this paper.

For each nucleotide position of each primer the diversity was determined for North-American strains of SARS-CoV-2 using the website nextstrain.org. Regions of the genome with high diversity scores contain more mutations, which may indicate that these regions evolve more rapidly than other regions. The highest “diversity score” for each primer set, as well as the lowest hairpin, homodimer, and heterodimer ΔGs for each primer set are shown in Table 4. Our primary criterion was to select primers for synthesis and wet lab testing that had the lowest possible sequence similarity with human genomic DNA or sequences from other coronaviruses, and acceptable diversity scores. The ΔG values for hairpin, homodimer, and heterodimer formation were already at acceptable levels according to the LAMP Designer software, and were found to be greater than −11 kcal/mol using Primer 3 software. We also calculated the same parameters for two LAMP primer sets that were previously reported (23) (Table 5).

TABLE 5
In silico Evaluation of Zhang SARS-CoV-2 LAMP Primer Sets (23)

| | | | | BLAST (other | | | | | |
|---|---|---|---|---|---|---|---|---|---|
| | | | | human | | | | | |
| | | BLAST (Human) | | coronaviruses) | | | | | |
| | Region | Avg. | Avg. | Avg. | Avg. | | | | |
| Primer | of | Query | % | Query | % | Diversity | ΔG^{c} | | |
| Set # | Genome | Coverageª | Identityª | Coverageª | Identityª | score^{b} | Hairpin | Homodimer | Heterodimer |
| ORF1aA | ORF1a | 64.7% | 99.1% | 90.2% | 97.4% | 0.129 | −2.2 | −12.2 | −4.9 |
| NA | N | 75.2% | 99.2% | 90.8% | 97.7% | 0.064 | −1.2 | −7.9 | −5.0 |

^{a}Average of the highest query coverage or percent identity found in the BLAST results for each primer set.
^{b}Highest diversity (entropy) score for North-American strains of SARS-CoV-2 using the database on nextstrain.org.
^{c}Primer3 software was used to determine the Gibbs free energy (ΔG) in kcal/mol for each primer (hairpin and homodimer) or primer pair (heterodimer). The lowest ΔG for each primer set is listed.

Isothermal Amplification of RNA Standard and RNA Extracted from Clinical Samples

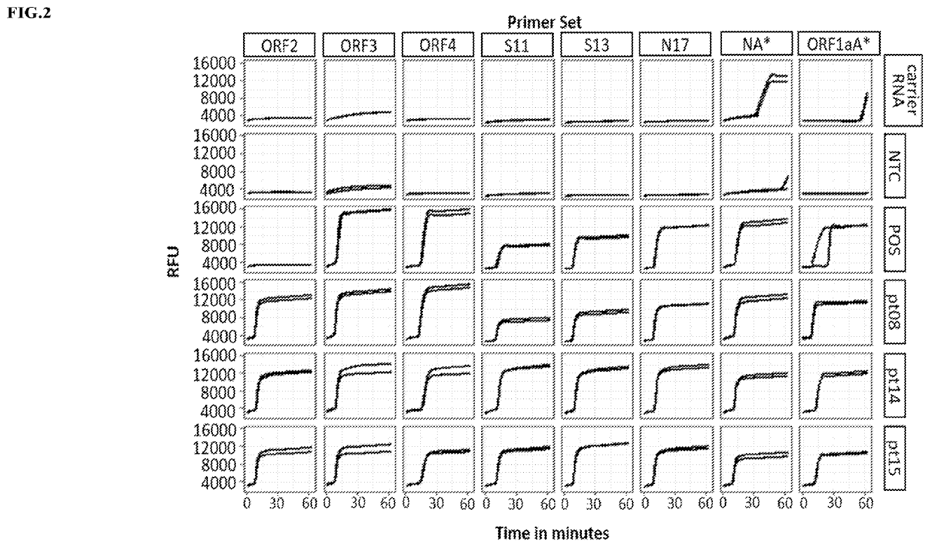

We first tested all six LAMP primer sets using 1000 copies of SARS-CoV-2 RNA standard (positive control) or with 5 μL of RNA extracted from saliva samples from patients who had previously tested positive via qRT-PCR. Except for the ORF2 primer set, all primer sets resulted in a positive detection signal for the RNA standard (FIG. 2 and Table 6). This was expected as the SARS-CoV-2 standard from Exact Diagnostics only contains RNA transcripts for the E, N, ORF1ab, RdRP and S genes, and does not include the 5′ UTR of the genome which several of the primers in the is set bind to. All six primer sets resulted in a positive detection signal for saliva samples from patients 8, 14 and 15, which were also positive in the qRT-PCR reference test. No signal was detected for the NTC controls (FIG. 2). The average time-to-threshold (Tt) values for each primer set are shown in Table 6. The ORF2, ORF3, and S13 primer sets were able to amplify the sample from patient 8 the in shortest amount of time, and this trend continued for the other two positive patient samples. Additionally, the threshold times of these three primer sets were below those that we measured with the previously published NA and ORF1aA primer sets (23) (Table 6 and FIG. 2).

TABLE 6
LAMP results for SARS-CoV-2 positive samples and
positive controls

| | Average Tt for Primer Set (minutes): | | | | | | | |
|---|---|---|---|---|---|---|---|---|
| | | | | | | | ORF1aA | NA |
| Sample | ORF2 | ORF3 | ORF4 | S11 | S13 | N17 | ⁽²³⁾ | ⁽²³⁾ |
| Positive | — | 9.0 | 15.5 | 10.9 | 8.4 | 9.8 | 35.1 | 10.5 |
| ctrl | | | | | | | | |
| Pt #8 | 6.7 | 7.4 | 12.4 | 9.3 | 7.5 | 8.3 | 10.5 | 8.5 |
| Pt #14 | 8.8 | 9.2 | 16.0 | 11.7 | 9.6 | 10.4 | 13.1 | 10.5 |
| Pt #15 | 8.0 | 8.5 | 14.9 | 10.7 | 8.5 | 9.1 | 12.5 | 9.3 |

Legend: —, no amplification observed; Average Tt, average threshold time in minutes (average of two technical replicates); * 1000 copies per reaction

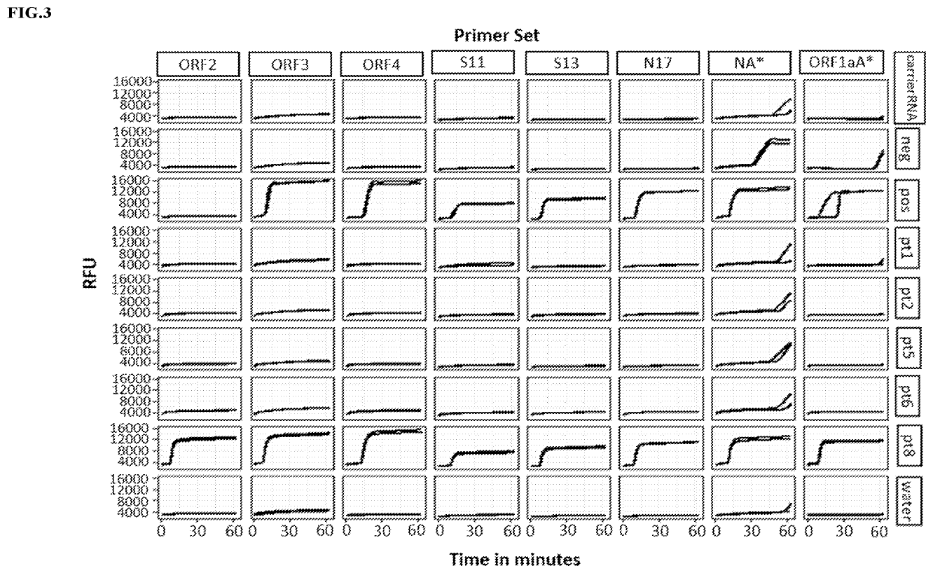

While patients 1, 2, 5, and 6 initially tested positive using nasopharyngeal swabs, saliva samples collected 11-50 days later, were negative by qRT-PCR. As shown in FIG. 3, the saliva samples from these patients also tested negative with using of our novel six LAMP primer sets (N17, ORF2, ORF3, ORF4 S11, and S13). However, unspecific false positive signals were obtained at late incubation times (>40 min.) when using the NA and ORF1aA primer sets published by others (23). Additionally, no amplification was observed when carrier RNA, which was added to all patient samples during RNA extraction, or genomic human DNA was used as a template (FIG. 3). In contrast, the NA primer set amplifies the carrier RNA, the human genomic DNA, plus the negative patient samples, while the ORF1aA primer set amplifies the negative control (FIG. 3).

### Limit of Detection

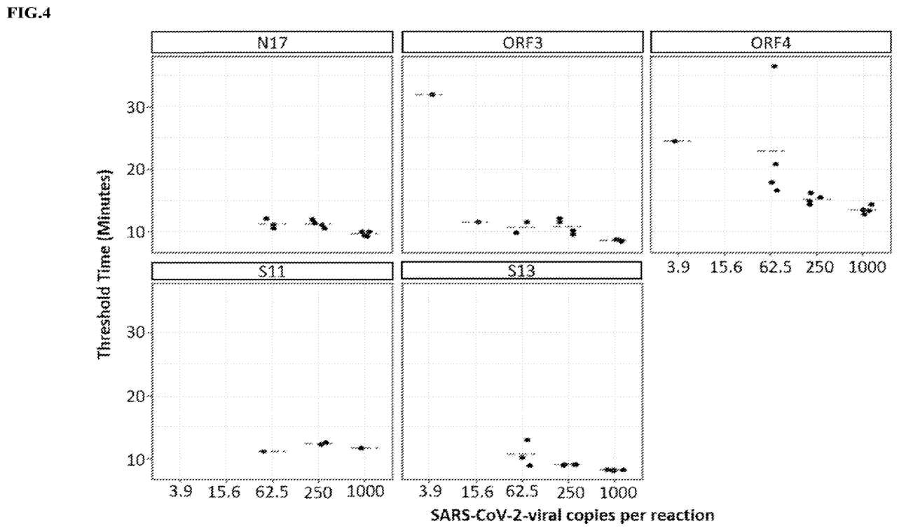

The limit of detection (LoD) of five out of the six novel LAMP primer sets was determined by RT-LAMP. Four-fold serial dilutions of the RNA standard, ranging from 200 copies per μL to <1 copy per μL (1000 to 3.9 copies per reaction) were tested in quadruplicate. No LoD testing was performed on the ORF2 primer set as it had previously been determined that the RNA standard was not amplified by this primer set (FIG. 2, Table 6, FIG. 3) as the standard does not contain the entire SARS-COV-2 genome. Among the other five primer sets, ORF4 showed the highest sensitivity, as all replicates were positive when 62.5 copies of RNA template were used. However, the ORF4 primer set also has the highest threshold times (FIG. 4). Furthermore, while the ORF2 primer set does not amplify the RNA standard, it efficiently amplified viral RNA extracted from the clinical samples. Because the ORF2 primer set can differentiate between the RNA standard and viral RNA, it may be very useful to include it in future assays as a control, since it would guard against potential contaminations from amplicons originating from a positive control. We plan to confirm these LoDs with 20 additional replicates, as 19 out of 20 replicates need to be detected for regulatory purposes.

TABLE 7
Limit of Detection of SARS-CoV-2 RNA standard by RT-LAMP

| | ORF3 | | ORF4 | | S11 | | S13 | | N17 | |
|---|---|---|---|---|---|---|---|---|---|---|
| RNA | Avg. | | Avg. | | Avg. | | Avg. | | Avg. | |
| COPIES | Tt | PR | Tt | PR | Tt | PR | Tt | PR | Tt | PR |
| 1000 | 8.6 | 4/4 | 13.5 | 4/4 | 10.8 | 4/4 | 8.1 | 4/4 | 9.7 | 4/4 |
| 250 | 10.9 | 2/4 | 15.2 | 4/4 | 12.3 | 2/4 | 9.0 | 4/4 | 11.3 | 4/4 |
| 62.5 | 10.7 | 2/4 | 22.9 | 4/4 | 11.1 | 1/4 | 10.6 | 3/4 | 11.2 | 3/4 |
| 15.6 | 11.5 | 1/4 | — | 0/4 | — | 0/4 | — | 0/4 | — | 0/4 |
| 3.9 | 32.0 | 1/4 | 24.4 | 1/4 | — | 0/4 | — | 0/4 | — | 0/4 |
| NTC | — | 0/4 | — | 0/4 | — | 0/4 | — | 0/4 | — | 0/4 |

Legend: NTC, non-template control; —, no amplification observed; Avg. Tt, average threshold time in minutes; PR, positive replicates.

### Example 4: Discussion

To create a rapid and sensitive LAMP-based SARS-CoV-2 diagnostic assay, we designed 17 novel SARS-CoV-2 LAMP primer sets. After in silico analysis, six primer sets were chosen together with two previously published primers (23) and custom synthesized for in vitro analysis. In addition to controls, seven clinical samples were tested by both qRT-PCR and RT-LAMP. All six of our novel LAMP primer sets showed 100% agreement with the qRT-PCR results, with four patient samples testing negative and three samples testing positive with a mean threshold time of 9.8±2.5 minutes across all six primer sets. In comparison, the qRT-PCR assay showed a mean cycle threshold time of 19.3±4.6 minutes. The ORF4 primer set had the lowest LOD (highest sensitivity). However, it also exhibited slower Tt values. Primer sets ORF3, S13 and N17 exhibited the fastest Tt values for most standard dilutions. The same sets and the ORF2 set had the best time-to-positive (Tt) for patient saliva samples.

In future work, we plan to evaluate a larger number of clinical samples to determine the full diagnostic potential, sensitivity, and specificity of our primer sets.

### Example 5: Lamp Detection of SARS-CoV-2 in Saliva for the Rapid Diagnosis of Covid-19

To explore the robustness of our assay and the potential for non-specific target amplification, we have tested our novel SARS-CoV-2 LAMP primer sets, ORF2, S13, and N17, on a respiratory control panel (EDX), containing common respiratory disease viruses and bacteria, including 5 other coronaviruses. According to the manufacturer, the panel contains 22 respiratory analytes in form of a combination of whole, intact virus and bacteria, that have been heat or chemically inactivated, and synthetic RNA transcripts.

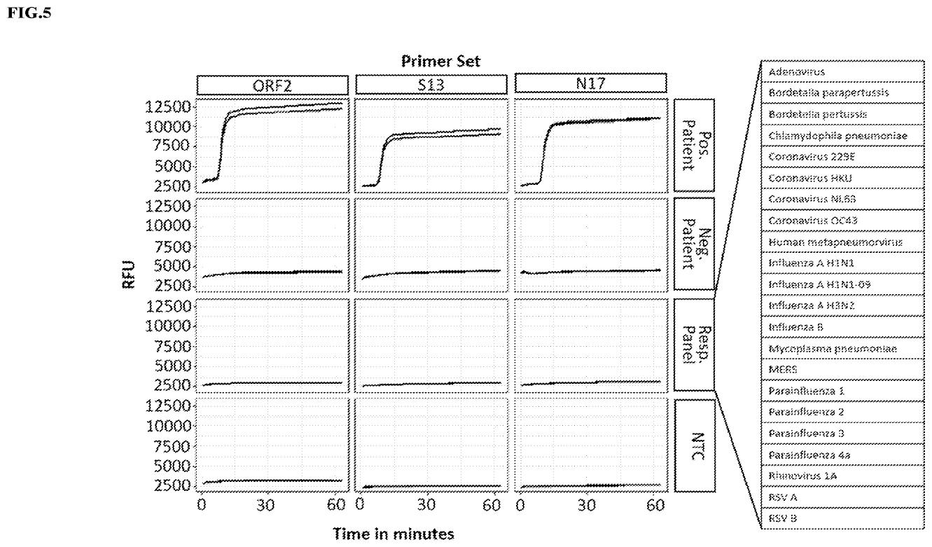

Our LAMP primer sets showed no amplification with the respiratory panel (FIG. 5). Only saliva samples from a confirmed COVID-19 patient produced positive results.

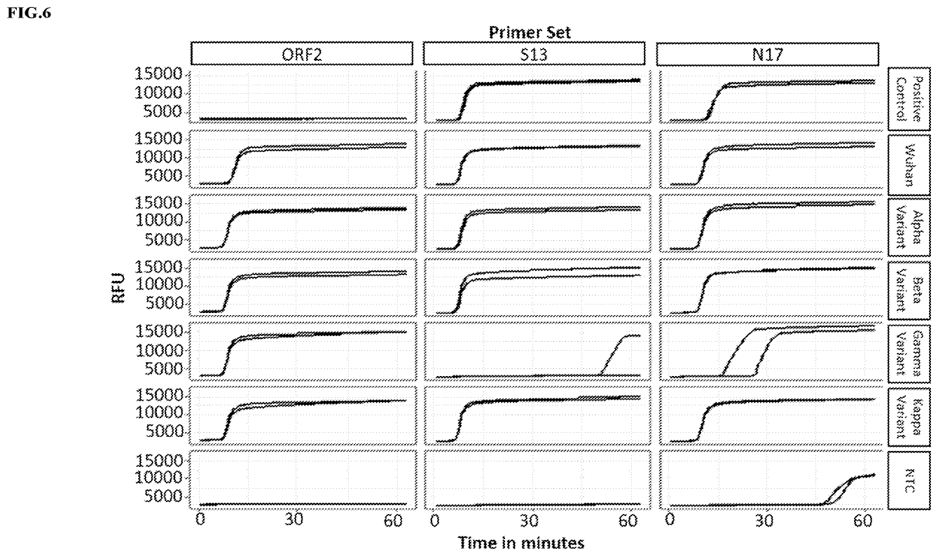

LAMP detection was also efficient and positive for known variants of SARS-CoV-2, including for the Wuhan strain, the variants α (alpha), β (beta), γ (gamma) and κ (kappa). S13 was the only primer set with less than satisfactory amplification of the γ-variant (FIG. 6). Standards for the currently fast spreading δ (delta) variant, were not available. However, sequence analysis predicts that our primers should amplify and detect the δ (delta) and λ (lambda) variant as well.

It was obvious that the N17 primer set had FIP primer mismatched the sequence of the gamma variant. Therefore, we created a N17m-FIP primer containing a mixed base to accommodate the known variants. The sequence of the new N17m-FIP primer is: ACCGTCACCACCACGAATTCTTAACACCAATAGCAGTCSAG, with the S being a mixture of G and C.

The novel N17m primer set allowed amplification for all other variants, as well, but showed some non-specific amplification with a negative control at long incubation times (>47 minutes, Table 8).

TABLE 8
Modified primer set N17m to efficiently amplify
variant SARS-CoV-2 viruses.

| Variant or Sample | Tt well #1 | Tt well #2 | Avg. Tt |
|---|---|---|---|
| Wuhan | 9.5 | 9.5 | 9.5 |
| UK (alpha) | 9.2 | 9.2 | 9.2 |
| South Africa (beta) | 9.0 | 9.1 | 9.1 |
| Brazil (gamma) | 9.8 | 9.8 | 9.8 |
| India (kappa) | 9.3 | 9.1 | 9.2 |
| EDX Resp. Panel | 47.0 | — | 47.0 |
| TE + EDX neg (NTC) | 53.4 | 51.1 | 52.2 |
| EDX Positive Control | 14.3 | 14.3 | 14.3 |

Legend: NTC = non-template control (negative)

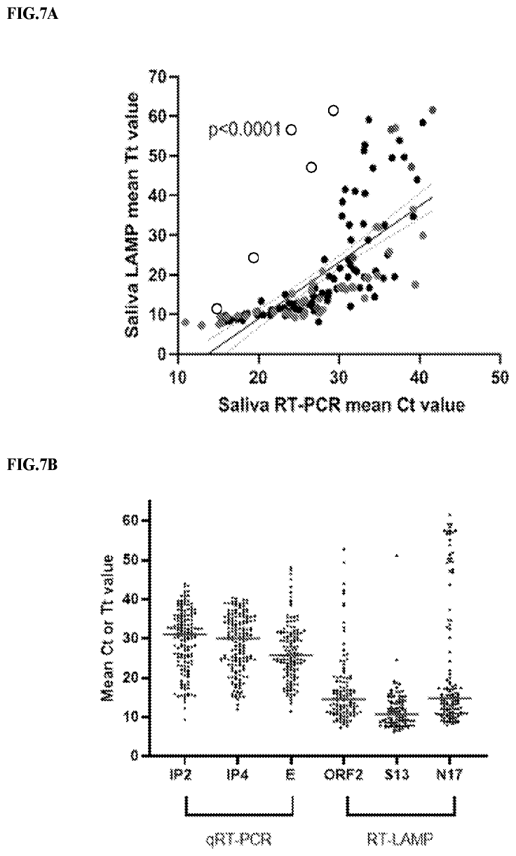

We analyzed over 83 clinical saliva specimens obtained from cancer patients who had a recent positive nasopharyngeal (NPS) swab RT PCR Covid-19 test performed at the City of Hope clinical lab. We conducted RT PCR and LAMP analysis on the RNA derived from these saliva samples. While NPS and saliva data do not always correlate—they are distinct specimens from different locations of the human body after all; there is, however, a very good correlation between RT PCR and LAMP results obtained from the same saliva samples (FIG. 7A-B). The data also shows that for relevant positive samples, the LAMP method is more than twice faster (FIG. 7 B).

### Example 6

| | Se- | | | | | | | | | | | | |
|---|---|---|---|---|---|---|---|---|---|---|---|---|---|
| Pri- | quence | | | | | | | | | | | GC | 3′ |
| mer | Defi- | Sequence | | | | Concen- | | | | | | Cl- | End |
| Set | nition | Length | Name | Quality | Primer | tration | Position | Length | Tm | GC% | Rating | amp | dG |
| 2 | All | 29,903 | F3 | | AATCTGTGTGGCTGTCAC | 5.3 | 81 | 18 | 59.6 | 50 | 85.3 | 1 | 0.1 |
| | | | B3 | | TCTGATAAGACCTCCTCCAC | 4.9 | 387 | 20 | 60 | 50 | 93.3 | 2 | −1.3 |
| | | | LoopF | | TAAGCAGCCTGCAGAAGATAG | 4 | 196 | 21 | 62 | 47.6 | 88.6 | 1 | 0.7 |
| | | | LoopB | | GTAAGATGGAGAGCCTTGTCC | 4.3 | 261 | 21 | 62.2 | 52.4 | 92.3 | 2 | 0.5 |
| | | | F2 | | TGACAGGACACGAGTAACT | 4.5 | 154 | 19 | 60 | 47.4 | 93.3 | 1 | −1 |
| | | | F1c | | AGATGTGCTGATGATCGGCTG | 4.3 | 234 | 21 | 64.9 | 52.4 | 93.3 | 3 | −2 |
| | | | B2 | | CTGAGTTGGACGTGTGTTT | 4.9 | 315 | 19 | 60.3 | 47.4 | 85.2 | 1 | −0.3 |
| | | | B1c | | GTCCGGGTGTGACCGAAAG | 4.7 | 242 | 19 | 65.3 | 63.2 | 84 | 1 | −1.9 |
| | | | Product | Best | | | | 162 | 73.1 | 51.2 | 89.5 | | |
| | Amplicon | | TGACAGGACACGAGTAACTCGTCTATCTTCTGCAGGCTGCTTACGGTTTCGTCCGTGTTGCAGCCGATCATCAGCACATCTAGGTTTT | | | | | | | | | | |
| | | | GTCCGGGTGTGACCGAAAGGTAAGATGGAGACCTTGTCCCTGGTTTCAACGAGAAAACACGTCCAACTCAG | | | | | | | | | | |
| 3 | All | 29,903 | F3 | | CTTGGCACTGATCCTTATGAA | 4.4 | 710 | 21 | 60.1 | 42.9 | 93.2 | 1 | −0.8 |
| | | | B3 | | GTTCCGTGTACCAAGCAA | 5.1 | 995 | 18 | 60 | 50 | 93.3 | 2 | −1.3 |
| | | | LoopF | | AAGCTCACGCATGAGTTCA | 4.7 | 796 | 19 | 62 | 47.4 | 92.4 | 1 | −0.5 |
| | | | LoopB | | CGTGCTGGTAAAGCTTCATG | 4.6 | 884 | 20 | 61.9 | 50 | 85.1 | 1 | −2.3 |
| | | | F2 | | AACATAGCAGTGGTGTTACC | 4.4 | 756 | 20 | 59.9 | 45 | 93.1 | 2 | −0.2 |
| | | | F1c | | ATCGACATAGCGAGTGTATGCC | 4.1 | 826 | 22 | 64.6 | 50 | 90.2 | w | −1.7 |
| | | | B2 | | GTCCAGTTGTTCGGACAA | 5.1 | 925 | 18 | 59.6 | 50 | 86.9 | 1 | −0.5 |
| | | | B1c | | GATGGCTACCCTCTTGAGTGC | 4.6 | 845 | 21 | 64.9 | 57.1 | 86.6 | N | −0.7 |
| | | | Product | Best | | | | 170 | 72.5 | 49.4 | 89 | | |
| 4 | All | 29,903 | F3 | | CAAGGTTACAAGAGTGTGAA | 4.1 | 2,765 | 20 | 59.7 | 40 | 91.4 | 1 | −0.6 |
| | | | B3 | | CTTCTTCTTCATCCTCATCTG | 5.1 | 3,045 | 21 | 59.9 | 42.9 | 93.2 | 1 | 0 |
| | | | LoopF | | TTACTTCTGTACCGAGTTCAAC | 4.3 | 2,849 | 22 | 62.2 | 40.9 | 92.8 | 1 | −0.2 |
| | | | LoopB | | AGTGGAGTATGGCTACATACT | 4.2 | 2,961 | 21 | 62 | 42.9 | 93.3 | 1 | −1.1 |
| | | | F2 | | GAGAAGTGCTCTGCCTATA | 4.7 | 2,828 | 19 | 60.1 | 47.4 | 93.1 | 1 | 0 |
| | | | F1c | | CACAACACAGGCGAACTCA | 4.6 | 2,872 | 19 | 65 | 52.6 | 93.3 | 1 | −0.7 |
| | | | B2 | | AACTCACCAGACTCATCAA | 4.7 | 2,988 | 19 | 60.1 | 42.1 | 93.1 | 1 | 0.2 |
| | | | B1c | | TTACTTACACCACTGGGCATTG | 4.3 | 2,980 | 22 | 65.1 | 45.5 | 86.6 | 1 | 0.5 |
| | | | Product | Best | | | | 179 | 69.4 | 41.3 | 92 | | |
| 11 | All | 29,903 | F3 | | CTTCTGCTAATCTTGCTGCTA | 4.8 | 24,621 | 21 | 60.2 | 42.9 | 92.9 | 2 | −0.2 |
| | | | B3 | | CATCACAGTTACCAGACACAA | 4.1 | 24,924 | 21 | 60.2 | 42.9 | 92.3 | 1 | −0.6 |
| | | | LoopF | | CCATGAGGTGCTGACTGAG | 4.7 | 24,720 | 19 | 60.2 | 57.9 | 92.6 | 1 | −0.5 |
| | | | LoopB | | ACTTTCCTCGTGAAGGTGTC | 4.8 | 24,825 | 20 | 62 | 50 | 86.7 | 1 | −0.1 |
| | | | F2 | | GGCTATCATCTTATGTCCTTCC | 4.6 | 24,698 | 22 | 60.1 | 45.5 | 93 | 2 | −1.2 |
| | | | F1c | | TGCAGGGACATAAGTCACATGC | 4 | 24,751 | 22 | 65.3 | 50 | 84.4 | 2 | −1.2 |
| | | | B2 | | GTGTGTGCCATTTGAAACAA | 4.4 | 24,846 | 20 | 60.1 | 40 | 86.6 | 1 | −0.3 |
| | | | B1c | | TTCACAACTGCTCCTGCCATT | 4.8 | 24,785 | 21 | 65 | 47.6 | 93.3 | 2 | −0.5 |
| | | | Product | Best | | | | 168 | 70.7 | 45.2 | 89.4 | | |
| 13 | All | 29,903 | F3 | | GGCTATCATCTTATGTCCTTCC | 4.6 | 24,698 | 22 | 60.1 | 45.5 | 93 | 2 | −1.2 |
| | | | B3 | | TACAACATCACAGTTACCAGAC | 4 | 24,928 | 22 | 60.1 | 40.9 | 93.2 | 1 | −0.7 |
| | | | LoopF | | GTGCAGGGACATAAGTCACA | 4.2 | 24,754 | 20 | 62.1 | 50 | 85.8 | 1 | −1 |
| | | | LoopB | | AGGTGTCTTTGTTTCAAATGGC | 4.2 | 24,838 | 22 | 62.2 | 40.9 | 86.1 | 3 | 1.3 |
| | | | F2 | | CCTCATGGTGTAGTCTTCTTG | 4.8 | 24,731 | 21 | 60.1 | 47.6 | 93.2 | 1 | −1.2 |
| | | | F1c | | AATGGCAGGAGCAGTTGTGAA | 3.9 | 24,785 | 21 | 65 | 47.6 | 93.3 | 1 | −0.5 |
| | | | B2 | | TGTGTTACAAACCAGTGTGT | 4.5 | 24,860 | 20 | 59.9 | 40 | 86.6 | 1 | −1.1 |
| | | | B1c | | TGGAAAAGCACACTTTCCTCGT | 4.2 | 24,814 | 22 | 65 | 45.5 | 79.7 | 2 | −1 |
| | | | Product | Best | | | | 149 | 69.6 | 43.6 | 87.5 | | |
| | Amplicon | | CCTCATGGTGTAGTCTTCTTGCATGTGACTTATGTCCCTGCACAAGAAAAGAACTTCACAACTGCTCCTGCCATTTGTCATGAT | | | | | | | | | | |
| | | | GGAAAAGCACACTTTCCTCGTGAAGGTGTCTTTGTTTCAAATGGCACACACTGGTTTGTAACACA | | | | | | | | | | |
| 17 | All | 29,903 | F3 | | TCAACATGGCAAGGAAGAC | 4.4 | 28,444 | 19 | 60.1 | 47.4 | 93.2 | 1 | −0.5 |
| | | | B3 | | TTGGTGTATTCAAGGCTCC | 5 | 28,682 | 19 | 59.5 | 47.4 | 88.7 | 2 | −1.8 |
| | | | LoopF | | GCTCTTCGGTAGTAGCCAATT | 4.5 | 28,522 | 21 | 62.2 | 47.6 | 92.3 | 1 | 0.4 |
| | | | LoopB | | GACTTCCCTATGGTGCTAACAA | 4.2 | 28,632 | 22 | 62.1 | 45.5 | 86.5 | 1 | −0.3 |
| | | | F2 | | TTAACACCAATAGCAGTCCAG | 4.2 | 28,494 | 21 | 60 | 42.9 | 93.3 | 2 | 0.7 |
| | | | F1c | | ACCGTCACCACCACGAATTC | 4.8 | 28,551 | 20 | 65.1 | 55 | 93 | 1 | 0.5 |
| | | | B2 | | ACCCATATGATGCCGTCT | 5.3 | 28,654 | 18 | 60.2 | 50 | 86.3 | 2 | −1.4 |
| | | | B1c | | CTAGGAACTGGGCCAGAAGC | 4.3 | 28,610 | 20 | 64.7 | 60 | 83.9 | 2 | −0.3 |
| | | | Product | Best | | | | 178 | 71.3 | 46.1 | 89.2 | | |
| 2 | All | 29,903 | F3 | | AATCTGTGTGGCTGTCAC | 5.3 | 81 | 18 | 59.6 | 50 | 85.3 | 1 | 0.1 |
| | | | B3 | | TCTGATAAGACCTCCTCCAC | 4.9 | 387 | 20 | 60 | 50 | 93.3 | 2 | −1.3 |
| | | | LoopF | | TAAGCAGCCTGCAGAAGATAG | 4 | 196 | 21 | 62 | 47.6 | 88.6 | 1 | 0.7 |
| | | | LoopB | | GTAAGATGGAGAGCCTTGTCC | 4.3 | 261 | 21 | 62.2 | 52.4 | 92.3 | 2 | 0.5 |
| | | | F2 | | TGACAGGACACGAGTAACT | 4.5 | 154 | 19 | 60 | 47.4 | 93.3 | 1 | −1 |
| | | | F1c | | AGATGTGCTGATGATCGGCTG | 4.3 | 234 | 21 | 64.9 | 52.4 | 93.3 | 3 | −2 |
| | | | B2 | | CTGAGTTGGACGTGTGTTT | 4.9 | 315 | 19 | 60.3 | 47.4 | 85.2 | 1 | −0.3 |
| | | | B1c | | GTCCGGGTGTGACCGAAAG | 4.7 | 242 | 19 | 65.3 | 63.2 | 84 | 1 | −1.9 |
| | | | Product | Best | | | | 162 | 73.1 | 51.2 | 89.5 | | |
| | Amplicon | | TGACAGGACACGAGTAACTCGTCTATCTTCTGCAGGCTGCTTACGGTTTCGTCCGTGTTGCAGCCGATCATCAGCACATCTA | | | | | | | | | | |
| | | | GGTTTTGTCCGGGTGTGACCGAAAGGTAAGATGGAGAGCCTTGTCCCTGGTTTCAACGAGAAAACACACGTCCAACTCAG | | | | | | | | | | |
| 3 | All | 29,903 | F3 | | CTTGGCACTGATCCTTATGAA | 4.4 | 710 | 21 | 60.1 | 42.9 | 93.2 | 1 | −0.8 |
| | | | B3 | | GTTCCGTGTACCAAGCAA | 5.1 | 995 | 18 | 60 | 50 | 93.3 | 2 | −1.3 |
| | | | LoopF | | AAGCTCACGCATGAGTTCA | 4.7 | 796 | 19 | 62 | 47.4 | 92.4 | 1 | −0.5 |
| | | | LoopB | | CGTGCTGGTAAAGCTTCATG | 4.6 | 884 | 20 | 61.9 | 50 | 85.1 | 1 | −2.3 |
| | | | F2 | | AACATAGCAGTGGTGTTACC | 4.4 | 756 | 20 | 59.9 | 45 | 93.1 | 2 | −0.2 |
| | | | F1c | | ATCGACATAGCGAGTGTATGCC | 4.1 | 826 | 22 | 64.6 | 50 | 90.2 | 3 | −1.7 |
| | | | B2 | | GTCCAGTTGTTCGGACAA | 5.1 | 925 | 18 | 59.6 | 50 | 86.9 | 1 | −0.5 |
| | | | B1c | | GATGGCTACCCTCTTGAGTGC | 4.6 | 845 | 21 | 64.9 | 57.1 | 86.6 | 2 | −0.7 |
| | | | Product | Best | | | | 170 | 72.5 | 49.4 | 89 | | |
| 4 | All | 29,903 | F3 | | CAAGGTTACAAGAGTGTGAA | 4.1 | 2,765 | 20 | 59.7 | 40 | 91.4 | 1 | −0.6 |
| | | | B3 | | CTTCTTCTTCATCCTCATCTG | 5.1 | 3,045 | 21 | 59.9 | 42.9 | 93.2 | 1 | 0 |
| | | | LoopF | | TTACTTCTGTACCGAGTTCAAC | 4.3 | 2,849 | 22 | 62.2 | 40.9 | 92.8 | 1 | −0.2 |
| | | | LoopB | | AGTGGAGTATGGCTACATACT | 4.2 | 2,961 | 21 | 62 | 42.9 | 93.3 | 1 | −1.1 |
| | | | F2 | | GAGAAGTGCTCTGCCTATA | 4.7 | 2,828 | 19 | 60.1 | 47.4 | 93.1 | 1 | 0 |
| | | | F1c | | CACAACACAGGCGAACTCA | 4.6 | 2,872 | 19 | 65 | 52.6 | 93.3 | 1 | −0.7 |
| | | | B2 | | AACTCACCAGACTCATCAA | 4.7 | 2,988 | 19 | 60.1 | 42.1 | 93.1 | 1 | 0.2 |
| | | | B1c | | TTACTTACACCACTGGGCATTG | 4.3 | 2,980 | 22 | 65.1 | 45.4 | 86.6 | 1 | 0.5 |
| | | | Product | Best | | | | 179 | 69.4 | 41.3 | 92 | | |

| | Se- | | | | | Self | Hair- | Hair- | Self | | Hair- | Hair- | Self | | | |
|---|---|---|---|---|---|---|---|---|---|---|---|---|---|---|---|---|
| | quence | Se- | | | | Dimer | pin | pin | Dimer | | pin | pin | Dimer | Dimer | | |
| Pri- | Defi- | quence | | | | dG | Dg(In- | Bond | bond | Run/ | dG | bond | dG | bond | | |
| mer | ni- | Len- | | Qual- | | (In- | ter- | (Inter- | (In- | Repeat | (3′ | (3′ | (3′ | (3′ | Ta- | Ba- |
| Set | tion | gth | Name | ity | Primer | ternal) | nal) | nal) | ternal) | Length | End) | End) | End) | End) | Opt | ses |
| 2 | All | 29,903 | F3 | | AATCTGTGTGGCTGTCAC | 0 | 0 | 0 | 0 | 3 | 0 | 0 | 0 | 0 | | |
| | | | B3 | | TCTGATAAGACCTCCTCCAC | 0 | 0 | 0 | 0 | 2 | 0 | 0 | 0 | 0 | | |
| | | | LoopF | | TAAGCAGCCTGCAGAAGATAG | −2.2 | −0.3 | 3 | 6 | 2 | 0 | 0 | 0 | 0 | | |
| | | | LoopB | | GTAAGATGGAGAGCCTTGTCC | 0 | 0 | 0 | 0 | 2 | 0 | 0 | 0 | 0 | | |
| | | | F2 | | TGACAGGACACGGTAACT | 0 | 0 | 0 | 0 | 2 | 0 | 0 | 0 | 0 | | |
| | | | F1c | | AGATGTGCTGATGATCGGCTG | 0 | 0 | 0 | 0 | 2 | 0 | 0 | 0 | 0 | | |
| | | | B2 | | CTGAGTTGGACGTGTGTTT | −1 | 0 | 0 | 4 | 3 | 0 | 0 | 0 | 0 | | |
| | | | B1c | | GTCCGGGTGTGACCGAAAG | −1.7 | −0.5 | 3 | 4 | 3 | 0 | O | 0 | 0 | | |
| | | | Product | Best | | | | | | | | | | | 54.2 | 0 |
| | | | TGACAGGACACGAGTAACTCGTCTATCTTCTGCAGGCTGCTTACGGTTTCGTCCGTGTTGCAGCCGATCATCAGCA | | | | | | | | | | | | | |
| | | | CATCTAGGTTTTGTCCGGGTGTGACCGAAAGGTAAGATGGAGAGCCTTGTCCCTGGTTTCAACGAGAAAACACAC | | | | | | | | | | | | | |
| | | | GTCCAACTCAG | | | | | | | | | | | | | |
| 3 | All | 29,903 | F3 | | CTTGGCACTGATCCTTATGAA | 0 | 0 | 0 | 0 | 2 | 0 | 0 | 0 | 0 | | |
| | | | B3 | | GTTCCGTGTACCAAGCAA | 0 | 0 | 0 | 0 | 2 | 0 | 0 | 0 | 0 | | |
| | | | LoopF | | AAGCTCACGCATGAGTTCA | −0.9 | 0 | 4 | 4 | 2 | 0 | 0 | 0 | 0 | | |
| | | | LoopB | | CGTGCTGGTAAAGCTTCATG | −1.5 | −0.1 | 3 | 6 | 3 | 0 | 0 | 0 | 0 | | |
| | | | F2 | | AACATAGCAGTGGTGTTACC | 0 | 0 | 0 | 0 | 2 | 0 | 0 | 0 | 0 | | |
| | | | F1c | | ATCGACATAGCGAGTGTATGCC | −0.7 | 0 | 3 | 4 | 2 | 0 | 0 | 0 | 0 | | |
| | | | B2 | | GTCCAGTTGTTCGGACAA | 0 | 0 | 0 | 0 | 2 | −0.5 | 5 | −0.5 | 4 | | |
| | | | B1c | | GATGGCTACCCTCTTGAGTGC | 0 | 0 | 0 | 0 | 3 | 0 | O | 0 | 0 | | |
| | | | Product | Best | | | | | | | | | | | 53.7 | 0 |
| 4 | All | 29,903 | F3 | | CAAGGTTACAAGAGTGTGAA | 0 | 0 | 0 | 0 | 2 | 0 | 0 | 0 | 0 | | |
| | | | B3 | | CTTCTTCTTCATCCTCATCTG | 0 | 0 | 0 | 0 | 2 | 0 | 0 | 0 | 0 | | |
| | | | LoopF | | TTACTTCTGTACCGAGTTCAAC | 0 | 0 | 0 | 0 | 2 | 0 | 0 | 0 | 0 | | |
| | | | LoopB | | AGTGGAGTATGGCTACATACT | 0 | 0 | 0 | 0 | 2 | 0 | 6 | 0 | 6 | | |
| | | | F2 | | GAGAAGTGCTCTGCCTATA | 0 | 0 | 0 | 0 | 2 | 0 | 0 | 0 | 0 | | |
| | | | F1c | | CACAACACAGGCGAACTCA | 0 | 0 | 0 | 0 | 2 | 0 | 0 | 0 | 0 | | |
| | | | B2 | | AACTCACCAGACTCATCAA | 0 | 0 | 0 | 0 | 2 | 0 | 0 | 0 | 0 | | |
| | | | B1c | | TTACTTACACCACTGGGCATTG | 0 | 0 | 0 | 0 | 3 | 0 | 0 | 0 | 0 | | |
| | | | Product | Best | | | | | | | | | | | 51.6 | 0 |
| 11 | All | 29,903 | F3 | | CTTCTGCTAATCTTGCTGCTA | 0 | 0 | 0 | 0 | 2 | 0 | 0 | 0 | 0 | | |
| | | | B3 | | CATCACAGTTACCAGACACAA | 0 | 0 | 0 | 0 | 2 | 0 | 0 | 0 | 0 | | |
| | | | LoopF | | CCATGAGGTGCTGACTGAG | 0 | 0 | 0 | 0 | 2 | 0 | 0 | 0 | 0 | | |
| | | | LoopB | | ACTTTCCTCGTGAAGGTGTC | 0 | 0 | 0 | 0 | 3 | 0 | 0 | 0 | 0 | | |
| | | | F2 | | GGCTATCATCTTATGTCCTTCC | 0 | 0 | 0 | 0 | 2 | 0 | 0 | 0 | 0 | | |
| | | | F1c | | TGCAGGGACATAAGTCACATGC | −1.1 | 0 | 0 | 4 | 3 | −0.3 | 3 | −0.3 | 3 | | |
| | | | B2 | | GTGTGTGCCATTTGAAACAA | 0 | 0 | 0 | 0 | 3 | 0 | 0 | 0 | 0 | | |
| | | | B1c | | TTCACAACTGCTCCTGCCATT | 0 | 0 | 0 | 0 | 2 | 0 | 0 | 0 | 0 | | |
| | | | Product | Best | | | | | | | | | | | 52.6 | |
| 13 | All | 29,903 | F3 | | GGCTATCATCTTATGTCCTTCC | 0 | 0 | 0 | 0 | 2 | 0 | 0 | 0 | 0 | | |
| | | | B3 | | TACAACATCACAGTTACCAGAC | 0 | 0 | 0 | 0 | 2 | 0 | 0 | 0 | 0 | | |
| | | | LoopF | | GTGCAGGGACATAAGTCACA | −1.1 | 0 | 0 | 0 | 3 | 0 | 0 | 0 | 0 | | |
| | | | LoopB | | AGGTGTCTTTGTTTCAAATGGC | 0 | 0 | 0 | 0 | 3 | 0 | 0 | 0 | 0 | | |
| | | | F2 | | CCTCATGGTGTAGTCTTCTTG | 0 | 0 | 0 | 0 | 2 | 0 | 0 | 0 | 0 | | |
| | | | F1c | | AATGGCAGGAGCAGTTGTGAA | 0 | 0 | 0 | 0 | 2 | 0 | 0 | 0 | 0 | | |
| | | | B2 | | TGTGTTACAAACCAGTGTGT | 0 | 0 | 0 | 0 | 3 | 0 | 0 | 0 | 0 | | |
| | | | B1c | | TGGAAAAGCACACTTTCCTCGT | −0.5 | −0.5 | 5 | 5 | 4 | 0 | 0 | 0 | 0 | | |
| | | | Product | Best | | | | | | | | | | | 51.8 | 0 |
| | Amp- | CCTCATGGTGTAGTCTTCTTGCATGTGACTTATGTCCCTGCACAAGAAAAGAACTTCACA | | | | | | | | | | | | | | |
| | licon | ACTGCTCCTGCCATTTGTCATGATGGAAAAGCACACTTTCCTCGTGAAGGTGTCTTTGTTT | | | | | | | | | | | | | | |
| | | CAAATGGCACACACTGGTTTGTAACACA | | | | | | | | | | | | | | |
| 17 | All | 29,903 | F3 | | TCAACATGGCAAGGAAGAC | 0 | 0 | 0 | 0 | 2 | 0 | 0 | 0 | 0 | | |
| | | | B3 | | TTGGTGTATTCAAGGCTCC | 0 | 0 | 0 | 0 | 2 | 0 | 0 | 0 | 0 | | |
| | | | LoopF | | GCTCTTCGGTAGTAGCCAATT | −0.1 | −0.1 | 3 | 3 | 2 | 0 | 0 | 0 | 0 | | |
| | | | LoopB | | GACTTCCCTATGGTGCTAACAA | 0 | 0 | 0 | 0 | 3 | 0 | 0 | 0 | 0 | | |
| | | | F2 | | TTAACACCAATAGCAGTCCAG | 0 | 0 | 0 | 0 | 2 | 0 | 0 | 0 | 0 | | |
| | | | F1c | | ACCGTCACCACCACGAATTC | −0.1 | −0.1 | 3 | 3 | 2 | 0 | 0 | 0 | 6 | | |
| | | | B2 | | ACCCATATGATGCCGTCT | 0 | 0 | 0 | 0 | 3 | 0 | 0 | 0 | 0 | | |
| | | | B1c | | CTAGGAACTGGGCCAGAAGC | −1.8 | 0 | 0 | 4 | 3 | 0 | 0 | 0 | 0 | | |
| | | | Product | Best | | | | | | | | | | | 52.9 | 0 |

### REFERENCES

1. Coronavirus disease 2019 (COVID-19) situation reports, situation report 201 World Health Organization.
2. COVID-19 Public Health Emergency of International Concern (PHEIC) Global research and innovation forum. World Health Organization; 2020 12 Feb. 2020.
3. Dhama K, Sharun K, Tiwari R, Dadar M, Malik Y S, Singh K P, et al. COVID-19, an emerging coronavirus infection: advances and prospects in designing and developing vaccines, immunotherapeutics, and therapeutics. Hum Vaccin Immunother. 2020; 16(6):1232-8.
4. Jean S S, Lee P I, Hsueh P R. Treatment options for COVID-19: The reality and challenges. J Microbiol Immunol Infect. 2020; 53(3):436-43.
5. Venter M, Richter K. Towards effective diagnostic assays for COVID-19: a review. 2020; 73(7):370-7.
6. Tang Y W, Schmitz J E, Persing D H, Stratton C W. Laboratory Diagnosis of COVID-19: Current Issues and Challenges. J Clin Microbiol. 2020; 58(6).
7. Radio N P. Despite Early Warnings, U.S. Took Months To Expand Swab Production For COVID-19 Test. 2020.
8. Wang H, Liu Q, Hu J, Zhou M, Yu M Q, Li K Y, et al. Nasopharyngeal Swabs Are More Sensitive Than Oropharyngeal Swabs for COVID-19 Diagnosis and Monitoring the SARS-CoV-2 Load. Front Med (Lausanne). 2020; 7:334.
9. Chan J F, Yuan S, Kok K H, To K K, Chu H, Yang J, et al. A familial cluster of pneumonia associated with the 2019 novel coronavirus indicating person-to-person transmission: a study of a family cluster. Lancet. 2020; 395(10223):514-23.
10. Wang W, Xu Y, Gao R, Lu R, Han K, Wu G, et al. Detection of SARS-CoV-2 in Different Types of Clinical Specimens. JAMA. 2020.
11. To K K W, Yip C C Y, Lai C Y W, Wong C K H, Ho D T Y, Pang P K P, et al. Saliva as a diagnostic specimen for testing respiratory virus by a point-of-care molecular assay: a diagnostic validity study. Clin Microbiol Infect. 2019; 25(3):372-8.
12. To K K, Lu L, Yip C C, Poon R W, Fung A M, Cheng A, et al. Additional molecular testing of saliva specimens improves the detection of respiratory viruses. Emerg Microbes Infect. 2017; 6(6):e49.
13. To K K, Tsang O T, Yip C C, Chan K H, Wu T C, Chan J M, et al. Consistent Detection of 2019 Novel Coronavirus in Saliva. Clin Infect Dis. 2020; 71(15):841-3.
14. Mahony J B. Detection of respiratory viruses by molecular methods. Clin Microbiol Rev. 2008; 21(4):716-47.
15. Corman V M, Landt O, Kaiser M, Molenkamp R, Meijer A, Chu D K, et al. Detection of 2019 novel coronavirus (2019-nCoV) by real-time RT-PCR. Euro Surveill. 2020; 25(3).
16. Notomi T, Okayama H, Masubuchi H, Yonekawa T, Watanabe K, Amino N, et al. Loop-mediated isothermal amplification of DNA. Nucleic Acids Res. 2000; 28(12):E63.
17. Nagamine K, Hase T, Notomi T, Accelerated reaction by loop-mediated isothermal amplification using loop primers. Mol Cell Probes. 2002; 16(3):223-9.
18. Kaneko H, Kawana T, Fukushima E, Suzutani T, Tolerance of loop-mediated isothermal amplification to a culture medium and biological substances. J Biochem Biophys Methods. 2007; 70(3):499-501.
19. Etievant S, Bal A, Escuret V, Brengel-Pesce K, Bouscambert M, Cheynet V, et al. Performance Assessment of SARS-CoV-2 PCR Assays Developed by WHO Referral Laboratories. J Clin Med. 2020; 9(6).
20. Mark Aaron Behlke K L, Richard Owczarzy, Joseph Alan Walder, Yong You, inventor; Integrated Dna Technologies, Inc., assignee. Modifications for antisense compounds patent WO2012033848A1. 2010.
21. Untergasser A, Cutcutache I, Koressaar T, Ye J, Faircloth B C, Remm M, et al. Primer3—new capabilities and interfaces. Nucleic Acids Res. 2012; 40(15):e115.
22. Koressaar T, Remm M. Enhancements and modifications of primer design program Primer3. Bioinformatics. 2007; 23(10):1289-91.
23. Zhang Y, Odiwuor N, Xiong J, Sun L, Nyaruaba R O, Wei H, et al. Rapid Molecular Detection of SARS-CoV-2 (COVID-19) Virus RNA Using Colorimetric LAMP. 2020:2020.02.26.20028373.
24. Huang W E, Lim B, Hsu C C, Xiong D, Wu W, Yu Y, et al. RT-LAMP for rapid diagnosis of coronavirus SARS-CoV-2. Microb Biotechnol. 2020; 13(4):950-61.
25. Lamb L E, Bartolone S N, Ward E, Chancellor M B. Rapid detection of novel coronavirus/Severe Acute Respiratory Syndrome Coronavirus 2 (SARS-CoV-2) by reverse transcription-loop-mediated isothermal amplification. PLoS One. 2020; 15(6):e0234682.
26. Yu L, Wu S, Hao X, Li X, Liu X, Ye S, et al. Rapid colorimetric detection of COVID-19 coronavirus using a reverse tran-scriptional loop-mediated isothermal amplification (RT-LAMP) diagnostic plat-form: iLACO. 2020:2020.02.20.20025874.
27. Yan C, Cui J, Huang L, Du B, Chen L, Xue G, et al. Rapid and visual detection of 2019 novel coronavirus (SARS-CoV-2) by a reverse transcription loop-mediated isothermal amplification assay. Clin Microbiol Infect. 2020; 26(6):773-9.
28. Park G S, Ku K, Baek S H, Kim S J, Kim S I, Kim B T, et al. Development of Reverse Transcription Loop-Mediated Isothermal Amplification Assays Targeting Severe Acute Respiratory Syndrome Coronavirus 2 (SARS-CoV-2). J Mol Diagn. 2020; 22(6):729-35.
29. Bhadra S, Riedel T E, Lakhotia S, Tran N D, Ellington A D. High-surety isothermal amplification and detection of SARS-CoV-2, including with crude enzymes. 2020:2020.04.13.039941.
30. Osterdahl M F, Lee K A, Ni Lochlainn M, Wilson S, Douthwaite S, Horsfall R, et al. Detecting SARS-CoV-2 at point of care: Preliminary data comparing Loop-mediated isothermal amplification (LAMP) to PCR. 2020:2020.04.01.20047357.
31. Lu R, Wu X, Wan Z, Li Y, Zuo L, Qin J, et al. Development of a Novel Reverse Transcription Loop-Mediated Isothermal Amplification Method for Rapid Detection of SARS-CoV-2. Virol Sin. 2020; 35(3):344-7.
32. Lee J Y H, Best N, McAuley J, Porter J L, Seemann T, Schultz M B, et al. Validation of a single-step, single-tube reverse transcription loop-mediated isothermal amplification assay for rapid detection of SARS-CoV-2 RNA. J Med Microbiol. 2020.
33. Baek Y H, Um J, Antigua K J C, Park J H, Kim Y, Oh S, et al. Development of a reverse transcription-loop-mediated isothermal amplification as a rapid early-detection method for novel SARS-CoV-2. Emerg Microbes Infect. 2020; 9(1):998-1007.
34. El-Tholoth M, Bau H H, Song J. A Single and Two-Stage, Closed-Tube, Molecular Test for the 2019 Novel Coronavirus (COVID-19) at Home, Clinic, and Points of Entry. ChemRxiv. 2020.
35. Meagher R J, Priye A, Light Y K, Huang C, Wang E. Impact of primer dimers and self-amplifying hairpins on reverse transcription loop-mediated isothermal amplification detection of viral RNA. Analyst. 2018; 143(8):1924-33.

INFORMAL SEQUENCE LISTING

SEQ ID NO: 1: AATCTGTGTGGCTGTCAC
SEQ ID NO: 2: TCTGATAAGACCTCCTCCAC
SEQ ID NO: 3:
AGATGTGCTGATGATCGGCTGTGACAGGACACGAGTAACT
SEQ ID NO: 4:
GTCCGGGTGTGACCGAAAGCTGAGTTGGACGTGTGTTT
SEQ ID NO: 5: TAAGCAGCCTGCAGAAGATAG
SEQ ID NO: 6: GTAAGATGGAGAGCCTTGTCC
SEQ ID NO: 7: CTTGGCACTGATCCTTATGAA
SEQ ID NO: 8: GTTCCGTGTACCAAGCAA
SEQ ID NO: 9:
ATCGACATAGCGAGTGTATGCCAACATAGCAGTGGTGTTACC
SEQ ID NO: 10:
GATGGCTACCCTCTTGAGTGCGTCCAGTTGTTCGGACAA
SEQ ID NO: 11: AAGCTCACGCATGAGTTCA
SEQ ID NO: 12: CGTGCTGGTAAAGCTTCATG
SEQ ID NO: 13: CAAGGTTACAAGAGTGTGAA
SEQ ID NO: 14: CTTCTTCTTCATCCTCATCTG
SEQ ID NO: 15:
CACAACACAGGCGAACTCAGAGAAGTGCTCTGCCTATA
SEQ ID NO: 16:
TTACTTACACCACTGGGCATTGAACTCACCAGACTCATCAA
SEQ ID NO: 17: TTACTTCTGTACCGAGTTCAAC
SEQ ID NO: 18: AGTGGAGTATGGCTACATACT
SEQ ID NO: 19: CTTCTGCTAATCTTGCTGCTA
SEQ ID NO: 20: CATCACAGTTACCAGACACAA
SEQ ID NO: 21:
TGCAGGGACATAAGTCACATGCGGCTATCATCTTATGTCCTTCC
SEQ ID NO: 22:
TTCACAACTGCTCCTGCCATTGTGTGTGCCATTTGAAACAA
SEQ ID NO: 23: CCATGAGGTGCTGACTGAG
SEQ ID NO: 24: ACTTTCCTCGTGAAGGTGTC
SEQ ID NO: 25: GGCTATCATCTTATGTCCTTCC
SEQ ID NO: 26: TACAACATCACAGTTACCAGAC
SEQ ID NO: 27:
AATGGCAGGAGCAGTTGTGAACCTCATGGTGTAGTCTTCTTG
SEQ ID NO: 28:
TGGAAAAGCACACTTTCCTCGTTGTGTTACAAACCAGTGTGT
SEQ ID NO: 29: GTGCAGGGACATAAGTCACA
SEQ ID NO: 30: AGGTGTCTTTGTTTCAAATGGC
SEQ ID NO: 31: TCAACATGGCAAGGAAGAC
SEQ ID NO: 32: TTGGTGTATTCAAGGCTCC
SEQ ID NO: 33:
ACCGTCACCACCACGAATTCTTAACACCAATAGCAGTCCAG
SEQ ID NO: 34:
CTAGGAACTGGGCCAGAAGCACCCATATGATGCCGTCT
SEQ ID NO: 35: GCTCTTCGGTAGTAGCCAATT
SEQ ID NO: 36: GACTTCCCTATGGTGCTAACAA

### P EMBODIMENTS

P Embodiment 1: A kit comprising a set of loop-mediated isothermal amplification (LAMP) primers comprising:
(i) a set of LAMP primers comprising four primers comprising nucleic acid sequences having at least 90% sequence identity to the sequences of SEQ ID NO:1, SEQ ID NO:2, SEQ ID NO:3 or SEQ ID NO:4;
(ii) a set of LAMP primers comprising four primers comprising nucleic acid sequences having at least 90% sequence identity to the sequences of SEQ ID NO:7, SEQ ID NO:8, SEQ ID NO:9 or SEQ ID NO:10;
(iii) a set of LAMP primers comprising four primers comprising nucleic acid sequences having at least 90% sequence identity to the sequences of SEQ ID NO:13, SEQ ID NO:14, SEQ ID NO:15 or SEQ ID NO:16;
(iv) a set of LAMP primers comprising four primers comprising nucleic acid sequences having at least 90% sequence identity to the sequences of SEQ ID NO:19, SEQ ID NO:20, SEQ ID NO:21 or SEQ ID NO:22;
(v) a set of LAMP primers comprising four primers comprising nucleic acid sequences having at least 90% sequence identity to the sequences of SEQ ID NO:25, SEQ ID NO:26, SEQ ID NO:27 or SEQ ID NO:28; or
(vi) a set of LAMP primers comprising four primers comprising nucleic acid sequences having at least 90% sequence identity to the sequences of SEQ ID NO:31, SEQ ID NO:32, SEQ ID NO:33 or SEQ ID NO:34.

P Embodiment 2. The kit of P embodiment 1, wherein said set of LAMP primers in (i) further comprises a set of LAMP primers comprising two primers comprising nucleic acid sequences having at least 90% sequence identity to the sequences of SEQ ID NO:5 or SEQ ID NO:6.

P Embodiment 3. The kit of P embodiment 1, wherein said set of LAMP primers in (ii) further comprises a set of LAMP primers comprising two primers comprising nucleic acid sequences having at least 90% sequence identity to the sequences of SEQ ID NO:11 or SEQ ID NO:12.

P Embodiment 4. The kit of P embodiment 1, wherein said set of LAMP primers in (iii) further comprises a set of LAMP primers comprising two primers comprising nucleic acid sequences having at least 90% sequence identity to the sequences of SEQ ID NO:17 or SEQ ID NO:18.

P Embodiment 5. The kit of P embodiment 1, wherein said set of LAMP primers in (iv) further comprises a set of LAMP primers comprising two primers comprising nucleic acid sequences having at least 90% sequence identity to the sequences of SEQ ID NO:23 or SEQ ID NO:24.

P Embodiment 6. The kit of P embodiment 1, wherein said set of LAMP primers in (iv) further comprises a set of LAMP primers comprising two primers comprising nucleic acid sequences having at least 90% sequence identity to the sequences of SEQ ID NO:29 or SEQ ID NO:30.

P Embodiment 7. The kit of P embodiment 1, wherein said set of LAMP primers in (iv) further comprises a set of LAMP primers comprising two primers comprising nucleic acid sequences having at least 90% sequence identity to the sequences of SEQ ID NO:35 or SEQ ID NO:36.

P Embodiment 8. The kit of any one of P embodiments 1-7, wherein said set of LAMP primers is specific for an SARS-CoV-2 nucleic acid.

P Embodiment 9. The kit of any one of P embodiments 1-8, wherein said SARS-CoV-2 nucleic acid encodes a 5′-UTR/ORF1ab, a spike protein, nucleocapsid gene or a functional fragment thereof.

P Embodiment 10. The kit of any one of P embodiments 1-9, further comprising a strand-displacement polymerase.

P Embodiment 11. The kit of P embodiment 10, wherein said strand-displacement polymerase is a Bst polymerase.

P Embodiment 12. The kit of any one of P embodiments 1-11, wherein said at least 90% sequence identity is 95%.

P Embodiment 13. The kit of any one of P embodiments 1-11, wherein said at least 90% sequence identity is 98%.

P Embodiment 14. The kit of any one of P embodiments 1-11, wherein said at least 90% sequence identity is 100%.

P Embodiment 15. A method of detecting a SARS-CoV-2 nucleic acid in a sample, said method comprising:
(a) contacting a sample with a set of loop-mediated isothermal amplification (LAMP) primers under conditions sufficient to amplify a SARS-CoV-2 nucleic acid, wherein said set of LAMP primers comprises:
(i) a set of LAMP primers comprising four primers comprising nucleic acid sequences having at least 90% sequence identity to the sequences of SEQ ID NO:1, SEQ ID NO:2, SEQ ID NO:3 or SEQ ID NO:4;
(ii) a set of LAMP primers comprising four primers comprising nucleic acid sequences having at least 90% sequence identity to the sequences of SEQ ID NO:7, SEQ ID NO:8, SEQ ID NO:9 or SEQ ID NO:10;
(iii) a set of LAMP primers comprising four primers comprising nucleic acid sequences having at least 90% sequence identity to the sequences of SEQ ID NO:13, SEQ ID NO:14, SEQ ID NO:15 or SEQ ID NO:16;
(iv) a set of LAMP primers comprising four primers comprising nucleic acid sequences having at least 90% sequence identity to the sequences of SEQ ID NO:19, SEQ ID NO:20, SEQ ID NO:21 or SEQ ID NO:22;
(v) a set of LAMP primers comprising four primers comprising nucleic acid sequences having at least 90% sequence identity to the sequences of SEQ ID NO:25, SEQ ID NO:26, SEQ ID NO:27 or SEQ ID NO:28; or
(vi) a set of LAMP primers comprising four primers comprising nucleic acid sequences having at least 90% sequence identity to the sequences of SEQ ID NO:31, SEQ ID NO:32, SEQ ID NO:33 or SEQ ID NO:34; thereby forming a SARS-CoV-2 amplification product; and
(b) detecting said SARS-CoV-2 amplification product, thereby detecting said SARS-CoV-2 nucleic acid in said sample.

P Embodiment 16. The method of P embodiment 15, wherein said set of LAMP primers in (i) further comprises a set of LAMP primers comprising two primers comprising nucleic acid sequences having at least 90% sequence identity to the sequences of SEQ ID NO:5 or SEQ ID NO:6.

P Embodiment 17. The method of P embodiment 15, wherein said set of LAMP primers in (ii) further comprises a set of LAMP primers comprising two primers comprising nucleic acid sequences having at least 90% sequence identity to the sequences of SEQ ID NO:11 or SEQ ID NO:12.

P Embodiment 18. The method of P embodiment 15, wherein said set of LAMP primers in (iii) further comprises a set of LAMP primers comprising two primers comprising nucleic acid sequences having at least 90% sequence identity to the sequences of SEQ ID NO:17 or SEQ ID NO:18.

P Embodiment 19. The method of P embodiment 15, wherein said set of LAMP primers in (iv) further comprises a set of LAMP primers comprising two primers comprising nucleic acid sequences having at least 90% sequence identity to the sequences of SEQ ID NO:23 or SEQ ID NO:24.

P Embodiment 20. The method of P embodiment 15, wherein said set of LAMP primers in (iv) further comprises a set of LAMP primers comprising two primers comprising nucleic acid sequences having at least 90% sequence identity to the sequences of SEQ ID NO:29 or SEQ ID NO:30.

P Embodiment 21. The method of P embodiment 15, wherein said set of LAMP primers in (iv) further comprises a set of LAMP primers comprising two primers comprising nucleic acid sequences having at least 90% sequence identity to the sequences of SEQ ID NO:35 or SEQ ID NO:36.

P Embodiment 22. The method of P embodiment 15, wherein said SARS-CoV-2 nucleic acid encodes a 5′-UTR/ORF1 ab, a spike protein, nucleocapsid gene or a functional fragment thereof.

P Embodiment 23. The method of any one of P embodiments 15-22, wherein said at least 90% sequence identity is 95%.

P Embodiment 24. The method of any one of P embodiments 15-22, wherein said at least 90% sequence identity is 98%.

P Embodiment 25. The method of any one of P embodiments 15-22, wherein said at least 90% sequence identity is 100%.

P Embodiment 26. The method of any one of P embodiments 15-25, wherein said sample is saliva.

What is claimed is:

1. A kit comprising a set of loop-mediated isothermal amplification (LAMP) primers comprising:
(i) a set of LAMP primers comprising four primers comprising nucleic acid sequences having at least 90% sequence identity to the sequences of SEQ ID NO:1, SEQ ID NO:2, SEQ ID NO:3 and SEQ ID NO:4;
(ii) a set of LAMP primers comprising four primers comprising nucleic acid sequences having at least 90% sequence identity to the sequences of SEQ ID NO:7, SEQ ID NO:8, SEQ ID NO:9 and SEQ ID NO:10;
(iii) a set of LAMP primers comprising four primers comprising nucleic acid sequences having at least 90% sequence identity to the sequences of SEQ ID NO:13, SEQ ID NO:14, SEQ ID NO:15 and SEQ ID NO:16;
(iv) a set of LAMP primers comprising four primers comprising nucleic acid sequences having at least 90% sequence identity to the sequences of SEQ ID NO:19, SEQ ID NO:20, SEQ ID NO:21 and SEQ ID NO:22;
(v) a set of LAMP primers comprising four primers comprising nucleic acid sequences having at least 90% sequence identity to the sequences of SEQ ID NO:25, SEQ ID NO:26, SEQ ID NO:27 and SEQ ID NO:28; or
(vi) a set of LAMP primers comprising four primers comprising nucleic acid sequences having at least 90% sequence identity to the sequences of SEQ ID NO:31, SEQ ID NO:32, SEQ ID NO:33 and SEQ ID NO:34.

2. The kit of claim 1, wherein said set of LAMP primers in (i) further comprises a set of LAMP primers comprising two primers comprising nucleic acid sequences having at least 90% sequence identity to the sequences of SEQ ID NO:5 and SEQ ID NO:6.

3. The kit of claim 1, wherein said set of LAMP primers in (ii) further comprises a set of LAMP primers comprising two primers comprising nucleic acid sequences having at least 90% sequence identity to the sequences of SEQ ID NO:11 and SEQ ID NO:12.

4. The kit of claim 1, wherein said set of LAMP primers in (iii) further comprises a set of LAMP primers comprising two primers comprising nucleic acid sequences having at least 90% sequence identity to the sequences of SEQ ID NO:17 and SEQ ID NO:18.

5. The kit of claim 1, wherein said set of LAMP primers in (iv) further comprises a set of LAMP primers comprising two primers comprising nucleic acid sequences having at least 90% sequence identity to the sequences of SEQ ID NO:23 and SEQ ID NO:24.

6. The kit of claim 1, wherein said set of LAMP primers in (iv) further comprises a set of LAMP primers comprising two primers comprising nucleic acid sequences having at least 90% sequence identity to the sequences of SEQ ID NO:29 and SEQ ID NO:30.

7. The kit of claim 1, wherein said set of LAMP primers in (iv) further comprises a set of LAMP primers comprising two primers comprising nucleic acid sequences having at least 90% sequence identity to the sequences of SEQ ID NO:35 and SEQ ID NO:36.

8. The kit of claim 1, wherein said set of LAMP primers is specific for an SARS-CoV-2 nucleic acid.

9. The kit of claim 8, wherein said SARS-CoV-2 nucleic acid encodes a 5′-UTR/ORF1ab, a spike protein, nucleocapsid gene or a functional fragment thereof.

10. The kit of claim 1, further comprising a strand-displacement polymerase.

11. The kit of claim 10, wherein said strand-displacement polymerase is a Bst polymerase.

12. The kit of claim 1, wherein said at least 90% sequence identity is 95%.

13. The kit of claim 1, wherein said at least 90% sequence identity is 98%.

14. The kit of claim 1, wherein said at least 90% sequence identity is 100%.

15. A method of detecting a SARS-CoV-2 nucleic acid in a sample, said method comprising:
(a) contacting a sample with a set of loop-mediated isothermal amplification (LAMP) primers under conditions sufficient to amplify a SARS-CoV-2 nucleic acid, wherein said set of LAMP primers comprises:
(i) a set of LAMP primers comprising four primers comprising nucleic acid sequences having at least 90% sequence identity to the sequences of SEQ ID NO:1, SEQ ID NO:2, SEQ ID NO:3 and SEQ ID NO:4;
(ii) a set of LAMP primers comprising four primers comprising nucleic acid sequences having at least 90% sequence identity to the sequences of SEQ ID NO:7, SEQ ID NO:8, SEQ ID NO:9 and SEQ ID NO:10;
(iii) a set of LAMP primers comprising four primers comprising nucleic acid sequences having at least 90% sequence identity to the sequences of SEQ ID NO:13, SEQ ID NO:14, SEQ ID NO:15 and SEQ ID NO:16;
(iv) a set of LAMP primers comprising four primers comprising nucleic acid sequences having at least 90% sequence identity to the sequences of SEQ ID NO:19, SEQ ID NO:20, SEQ ID NO:21 and SEQ ID NO:22;
(v) a set of LAMP primers comprising four primers comprising nucleic acid sequences having at least 90% sequence identity to the sequences of SEQ ID NO:25, SEQ ID NO:26, SEQ ID NO:27 and SEQ ID NO:28; or
(vi) a set of LAMP primers comprising four primers comprising nucleic acid sequences having at least 90% sequence identity to the sequences of SEQ ID NO:31, SEQ ID NO:32, SEQ ID NO:33 and SEQ ID NO:34; thereby forming a SARS-CoV-2 amplification product; and
(b) detecting said SARS-CoV-2 amplification product, thereby detecting said SARS-CoV-2 nucleic acid in said sample.

16. The method of claim 15, wherein said set of LAMP primers in (i) further comprises a set of LAMP primers comprising two primers comprising nucleic acid sequences having at least 90% sequence identity to the sequences of SEQ ID NO:5 and SEQ ID NO:6.

17. The method of claim 15, wherein said set of LAMP primers in (ii) further comprises a set of LAMP primers comprising two primers comprising nucleic acid sequences having at least 90% sequence identity to the sequences of SEQ ID NO:11 and SEQ ID NO:12.

18. The method of claim 15, wherein said set of LAMP primers in (iii) further comprises a set of LAMP primers comprising two primers comprising nucleic acid sequences having at least 90% sequence identity to the sequences of SEQ ID NO:17 and SEQ ID NO:18.

19. The method of claim 15, wherein said set of LAMP primers in (iv) further comprises a set of LAMP primers comprising two primers comprising nucleic acid sequences having at least 90% sequence identity to the sequences of SEQ ID NO:23 and SEQ ID NO:24.

20. The method of claim 15, wherein said set of LAMP primers in (iv) further comprises a set of LAMP primers comprising two primers comprising nucleic acid sequences having at least 90% sequence identity to the sequences of SEQ ID NO:29 and SEQ ID NO:30.

21. The method of claim 15, wherein said set of LAMP primers in (iv) further comprises a set of LAMP primers comprising two primers comprising nucleic acid sequences having at least 90% sequence identity to the sequences of SEQ ID NO:35 and SEQ ID NO:36.

22. The method of claim 15, wherein said SARS-CoV-2 nucleic acid encodes a 5′-UTR/ORF1ab, a spike protein, nucleocapsid gene or a functional fragment thereof.

23. The method of claim 15, wherein said at least 90% sequence identity is 95%.

24. The method of claim 15, wherein said at least 90% sequence identity is 98%.

25. The method of claim 15, wherein said at least 90% sequence identity is 100%.

26. The method of claim 15, wherein said sample is saliva.


SEQUENCE LISTING (47 sequences)

SEQ ID NO: 1 - DNA, 18 bp, Artificial Sequence
  Synthetic polynucleotide
aatctgtgtggctgtcac

SEQ ID NO: 2 - DNA, 20 bp, Artificial Sequence
  Synthetic polynucleotide
tctgataagacctcctccac

SEQ ID NO: 3 - DNA, 40 bp, Artificial Sequence
  Synthetic polynucleotide
agatgtgctgatgatcggctgtgacaggacacgagtaact

SEQ ID NO: 4 - DNA, 38 bp, Artificial Sequence
  Synthetic polynucleotide
gtccgggtgtgaccgaaagctgagttggacgtgtgttt

SEQ ID NO: 5 - DNA, 21 bp, Artificial Sequence
  Synthetic polynucleotide
taagcagcctgcagaagatag

SEQ ID NO: 6 - DNA, 21 bp, Artificial Sequence
  Synthetic polynucleotide
gtaagatggagagccttgtcc

SEQ ID NO: 7 - DNA, 21 bp, Artificial Sequence
  Synthetic polynucleotide
cttggcactgatccttatgaa

SEQ ID NO: 8 - DNA, 18 bp, Artificial Sequence
  Synthetic polynucleotide
gttccgtgtaccaagcaa

SEQ ID NO: 9 - DNA, 42 bp, Artificial Sequence
  Synthetic polynucleotide
atcgacatagcgagtgtatgccaacatagcagtggtgttacc

SEQ ID NO: 10 - DNA, 39 bp, Artificial Sequence
  Synthetic polynucleotide
gatggctaccctcttgagtgcgtccagttgttcggacaa

SEQ ID NO: 11 - DNA, 19 bp, Artificial Sequence
  Synthetic polynucleotide
aagctcacgcatgagttca

SEQ ID NO: 12 - DNA, 20 bp, Artificial Sequence
  Synthetic polynucleotide
cgtgctggtaaagcttcatg

SEQ ID NO: 13 - DNA, 20 bp, Artificial Sequence
  Synthetic polynucleotide
caaggttacaagagtgtgaa

SEQ ID NO: 14 - DNA, 21 bp, Artificial Sequence
  Synthetic polynucleotide
cttcttcttcatcctcatctg

SEQ ID NO: 15 - DNA, 38 bp, Artificial Sequence
  Synthetic polynucleotide
cacaacacaggcgaactcagagaagtgctctgcctata

SEQ ID NO: 16 - DNA, 41 bp, Artificial Sequence
  Synthetic polynucleotide
ttacttacaccactgggcattgaactcaccagactcatcaa

SEQ ID NO: 17 - DNA, 22 bp, Artificial Sequence
  Synthetic polynucleotide
ttacttctgtaccgagttcaac

SEQ ID NO: 18 - DNA, 21 bp, Artificial Sequence
  Synthetic polynucleotide
agtggagtatggctacatact

SEQ ID NO: 19 - DNA, 21 bp, Artificial Sequence
  Synthetic polynucleotide
cttctgctaatcttgctgcta

SEQ ID NO: 20 - DNA, 21 bp, Artificial Sequence
  Synthetic polynucleotide
catcacagttaccagacacaa

SEQ ID NO: 21 - DNA, 44 bp, Artificial Sequence
  Synthetic polynucleotide
tgcagggacataagtcacatgcggctatcatcttatgtccttcc

SEQ ID NO: 22 - DNA, 41 bp, Artificial Sequence
  Synthetic polynucleotide
ttcacaactgctcctgccattgtgtgtgccatttgaaacaa

SEQ ID NO: 23 - DNA, 19 bp, Artificial Sequence
  Synthetic polynucleotide
ccatgaggtgctgactgag

SEQ ID NO: 24 - DNA, 20 bp, Artificial Sequence
  Synthetic polynucleotide
actttcctcgtgaaggtgtc

SEQ ID NO: 25 - DNA, 22 bp, Artificial Sequence
  Synthetic polynucleotide
ggctatcatcttatgtccttcc

SEQ ID NO: 26 - DNA, 22 bp, Artificial Sequence
  Synthetic polynucleotide
tacaacatcacagttaccagac

SEQ ID NO: 27 - DNA, 42 bp, Artificial Sequence
  Synthetic polynucleotide
aatggcaggagcagttgtgaacctcatggtgtagtcttcttg

SEQ ID NO: 28 - DNA, 42 bp, Artificial Sequence
  Synthetic polynucleotide
tggaaaagcacactttcctcgttgtgttacaaaccagtgtgt

SEQ ID NO: 29 - DNA, 20 bp, Artificial Sequence
  Synthetic polynucleotide
gtgcagggacataagtcaca

SEQ ID NO: 30 - DNA, 22 bp, Artificial Sequence
  Synthetic polynucleotide
aggtgtctttgtttcaaatggc

SEQ ID NO: 31 - DNA, 19 bp, Artificial Sequence
  Synthetic polynucleotide
tcaacatggcaaggaagac

SEQ ID NO: 32 - DNA, 19 bp, Artificial Sequence
  Synthetic polynucleotide
ttggtgtattcaaggctcc

SEQ ID NO: 33 - DNA, 41 bp, Artificial Sequence
  Synthetic polynucleotide
accgtcaccaccacgaattcttaacaccaatagcagtccag

SEQ ID NO: 34 - DNA, 38 bp, Artificial Sequence
  Synthetic polynucleotide
ctaggaactgggccagaagcacccatatgatgccgtct

SEQ ID NO: 35 - DNA, 21 bp, Artificial Sequence
  Synthetic polynucleotide
gctcttcggtagtagccaatt

SEQ ID NO: 36 - DNA, 22 bp, Artificial Sequence
  Synthetic polynucleotide
gacttccctatggtgctaacaa

SEQ ID NO: 37 - DNA, 41 bp, Artificial Sequence
  Synthetic polynucleotide
  variation (39)..(39): S is a G or C
accgtcaccaccacgaattcttaacaccaatagcagtcsag

SEQ ID NO: 38 - DNA, 41 bp, Artificial Sequence
  Synthetic polynucleotide
accgtcaccaccacgaattcttaacaccaatagcagtcgag

SEQ ID NO: 39 - DNA, 18 bp, Artificial Sequence
  Synthetic polynucleotide
atgagcttagtcctgttg

SEQ ID NO: 40 - DNA, 18 bp, Artificial Sequence
  Synthetic polynucleotide
ctccctttgttgtgttgt

SEQ ID NO: 41 - DNA, 21 bp, Artificial Sequence
  Synthetic polynucleotide
  misc_feature (9)..(10): napthylene-azo compound linkage
agatgtcttgtgctgccggta

SEQ ID NO: 42 - DNA, 19 bp, Artificial Sequence
  Synthetic polynucleotide
ggtaactggtatgatttcg

SEQ ID NO: 43 - DNA, 20 bp, Artificial Sequence
  Synthetic polynucleotide
ctggtcaaggttaatatagg

SEQ ID NO: 44 - DNA, 19 bp, Artificial Sequence
  Synthetic polynucleotide
  misc_feature (9)..(10): napthylene-azo compound linkage
tcatacaaaccacgccagg

SEQ ID NO: 45 - DNA, 26 bp, Artificial Sequence
  Synthetic polynucleotide
acaggtacgttaatagttaatagcgt

SEQ ID NO: 46 - DNA, 22 bp, Artificial Sequence
  Synthetic polynucleotide
atattgcagcagtacgcacaca

SEQ ID NO: 47 - DNA, 26 bp, Artificial Sequence
  Synthetic polynucleotide
  misc_feature (9)..(10): napthylene-azo compound linkage
acactagccatccttactgcgcttcg